(19) United States
(12) Patent Application Publication
(10) Pub. No.: US 2026/0306057 A1
(43) Pub. Date: Oct. 8, 2026

(54) ULTRASOUND COUPLING AGENT

(71) Applicant: Sonoclear AS, Oslo (NO)

(72) Inventor: Peter BALMFORTH, Oslo (NO)

(21) Appl. No.: 19/485,156

(22) PCT Filed: May 24, 2024
(86) PCT No.: PCT/IB2024/055100

§ 371 Date: Nov. 17, 2025

Related U.S. Application Data
(60) Provisional application No. 63/468,617, filed on May 24, 2023.

(51) Int. Cl. A61B 8/00; A61B 8/08; A61B 8/12; A61K 49/22

(52) U.S. Cl. CPC A61B 8/4281; A61B 8/0808; A61B 8/12; A61B 8/445; A61K 49/226

(57) ABSTRACT

Provided herein are ultrasound coupling agents comprising a triglyceride and a phospholipid and methods of using them for ultrasound imaging of a subject. The ultrasound imaging may be invasive ultrasound imaging. Also provided herein are methods of preparing ultrasound coupling agents and methods of formulating ultrasound coupling agents.

### CROSS-REFERENCE TO RELATED APPLICATIONS

This application claims the benefit of priority to U.S. Application No. 63/468,617, filed on May 24, 2023, which is incorporated by reference herein in its entirety for all purposes.

### BACKGROUND

Ultrasound imaging is widely used in medical examination to obtain real-time visualization of tissue and is used in various clinical fields. To ensure proper contact between the transducer/ultrasound probe and the skin/tissue to be examined an ultrasound coupling agent, which is typically a gel or liquid, is used. The ultrasound coupling agent is used to avoid air pockets between the transducer/probe and tissue, and to facilitate a good acoustic coupling at the interface between the ultrasound transducer and the tissue to be imaged.

Ultrasound imaging is often used in invasive procedures, such as during surgery or during an invasive diagnostic procedure. For example, ultrasound imaging may be used for imaging of tumors in brain surgery. In this case, ultrasound imaging may be used to locate the tumor and anatomical structures, as well as to identify residual tumor during surgery. Ultrasound imaging may also be used to make sure that the damaged tissue, such as a tumor, is completely removed, and that unnecessary resection of healthy tissue is avoided.

Ideally, high image quality should be sustained throughout the medical procedure in order to monitor the progress of that procedure (e.g. of surgery for tumor resection). However, surgery may cause image noise and thus lead to an inaccurate display of the relevant part of the anatomy in the ultrasound images.

The term artifact is used in medical imaging to describe any part of the image that does not accurately represent the anatomy of the subject being investigated. It is well known that ultrasound imaging is prone to several different types of artifacts which may lead to adverse outcomes for the subject. For example, when using ultrasound in brain tumor surgery, the presence of artifacts may interfere with the surgeon's interpretation of the images.

One artifact commonly encountered when using ultrasound for the invasive imaging of the body is an enhanced (anomalously bright) signal appearing below a fluid-filled cavity. The brightness enhancement of tissue located beneath fluid filled spaces has been observed in ultrasound imaging of cysts, blood vessels or other fluid filled spaces. Because of this apparent enhancement of the reflected echo, this frequently encountered image artifact is often referred to as a brightness artifact (sometimes also referred to as an “enhancement artefact”, “or “bright rim effect”). This type of artefact is a result of the difference in attenuation of acoustic waves by the fluid in the cavity and by the surrounding biological tissue.

For example, during brain tumor surgery a resection cavity is typically filled with saline water as an ultrasound coupling agent before ultrasound imaging, to enable propagation of sound and to prevent air artifacts. The difference in attenuation between brain and isotonic saline may cause artifacts that degrade the ultrasound images, potentially affecting surgical accuracy (e.g. resection grades and safety). The acoustic waves travel through the cavity filled with saline water before reaching the biological tissue. The attenuation of acoustic waves in saline water is very low compared to the attenuation of acoustic waves in biological tissue. The attenuation coefficient (a) for water is 0.0022 dB/(MHz*cm) while for e.g. brain it is reportedly measured by various groups to be within approximately 0.4-1.0 (Duck FA, In Physical properties of tissue, Academic Press, LTD).

A major component of the attenuation of sound in tissue is caused by absorption, in which part of the acoustic energy is converted to heat. Scattering also contributes to the attenuation of acoustic waves. These combined effects cause the acoustic waves propagating in saline water to have higher amplitudes than acoustic waves propagating an equal distance in biological tissue. The total attenuation is estimated by the equation:

Attenuation [dB]=α[dB/(MHz*cm)]*l[cm]*f[MHz]

Wherein α is the attenuation coefficient of the medium;
l is the medium length (or propagating distance); and
f is the frequency of the transmitted ultrasound wave.

For example, selecting a frequency of 8 MHz and a propagating distance of 10 cm, and assuming an attenuation coefficient of 0.8 for brain tissue this will result in an attenuation of 0.18 dB for ultrasound propagating in water and an attenuation of 64 dB for waves propagating in brain tissue.

This difference in attenuation can generate noise in the ultrasound images e.g. when ultrasound is used intraoperatively in brain tumor surgery. The ultrasound waves transmitted through the water-filled resection cavity will have a large amplitude when arriving at the cavity walls, due to the low attenuation of water. Thus, the sound waves reflected from the cavity wall will also have relatively high amplitudes. Further, the sound waves propagating further into the tissue will have relatively high amplitudes. In comparison to sound waves that have propagated entirely in brain tissue, with a relatively high attenuation coefficient, these transmitted and reflected waves that have travelled through saline will be less damped and thereby have significantly higher amplitudes. The fluctuations in intensity observed in the ultrasound images make it very difficult to interpret the images. The bright rim observed at the cavity wall may mask the presence of residual tumor, or the high intensity regions extending from the cavity wall may be interpreted as hyperechoic tumor when it is actually normal brain tissue.

The presence of the hyperechoic rim in ultrasound imaging of a resection cavity is described in several papers regarding the use of ultrasound in brain tumor surgery. This enhanced signal appearing below the fluid filled cavity in the ultrasound images is regarded as one of the major imaging artifacts encountered in preoperative ultrasound imaging.

There is a need in the art for new aqueous ultrasound coupling agents that are safe, stable, and do not lead to imaging artifacts.

### SUMMARY OF THE INVENTION

Provided herein is an aqueous ultrasound coupling agent consisting of: a) from about 60.0 to about 80.0 g/L of at least one triglyceride; b) from about 3.2 to about 5.2 g/L of at least one phospholipid; c) water; d) a pH buffer; and e) glycerol; wherein the ultrasound coupling agent has an attenuation constant from about 0.15 dB/(MHz*cm) to about 0.60 dB/(MHz*cm) or from about 0.002 to about 1.0 dB/(MHz*cm).

Provided here in an aqueous ultrasound coupling agent consisting of: a) at least one triglyceride; b) at least one phospholipid; c) water, d) pH buffer; and e) glycerol; wherein the ultrasound coupling agent has an attenuation constant from about 0.15 dB/(MHz*cm) to about 0.60 dB/(MHz*cm) or from about 0.002 to about 1.0 dB/(MHz*cm); and wherein the ratio of the at least one triglyceride to the at least one phospholipid is from about 13:1 to about 20:1 by weight.

Provided herein is an aqueous ultrasound coupling agent consisting of: a) at least one triglyceride, wherein the at least one triglyceride is present at about 7% w/v; b) at least one phospholipid, wherein the at least one phospholipid is present at about 0.4% w/v; c) water, wherein the water is present at about 92% w/v; d) pH buffer; and e) glycerol.

Provided herein is an aqueous ultrasound coupling agent comprising: a) from about 60.0 g/L to about 80.0 g/L of at least one triglyceride; b) from about 3.2 g/L to 5.2 g/L of at least one phospholipid, wherein the coupling agent does not include a stabilizer and does not include sodium chloride, and wherein the ultrasound coupling agent has an attenuation constant from about 0.15 dB/(MHz*cm) to about 0.60 dB/(MHz*cm) or from about 0.002 to about 1.0 dB/(MHz*cm).

Provided herein is an aqueous ultrasound coupling agent comprising: a) at least one triglyceride; and b) at least one phospholipid; wherein the coupling agent does not include a stabilizer and does not include sodium chloride; wherein the ultrasound coupling agent has an attenuation constant from about 0.15 dB/(MHz*cm) to about 0.60 dB/(MHz*cm) or from about 0.002 to about 1.0 dB/(MHz*cm); and wherein the ratio of the at least one triglyceride to the at least one phospholipid is from about 13:1 to about 20:1 by weight.

Provided herein is an aqueous ultrasound coupling agent comprising: a) at least one triglyceride, wherein the at least one triglyceride is present at about 7% w/v; and b) at least one phospholipid, wherein the at least one phospholipid is present at about 0.4% w/v; wherein the coupling agent does not include a stabilizer and does not include sodium chloride; and wherein the ultrasound coupling agent has an attenuation constant from about 0.15 dB/(MHz*cm) to about 0.60 dB/(MHz*cm) or from about 0.002 to about 1.0 dB/(MHz*cm).

In embodiments, the aqueous ultrasound coupling agents provided herein comprise about 70 g/L of at least one triglyceride.

In embodiments, the at least one triglyceride of the aqueous ultrasound coupling agents provided herein is selected from one or more of soybean oil, olive oil, or fish oil. In embodiments, the at least one triglyceride of the aqueous ultrasound coupling agents provided herein is soybean oil.

In embodiments, the aqueous ultrasound coupling agents provided herein comprise at least one humectant. In embodiments, the at least one humectant is glycerol.

In embodiments, the aqueous ultrasound coupling agents provided herein do not comprise PS80.

In embodiments, the aqueous ultrasound coupling agents provided herein have a pH from about 6.5 to about 8.5. In embodiments, the aqueous ultrasound coupling agents provided herein have a pH of about 7.3.

In embodiments, the aqueous ultrasound coupling agents provided herein have an osmolality from 270-330 mOsm/L.

In embodiments, the aqueous ultrasound coupling agents provided herein have an average droplet size of about 285 nm±20%, wherein the average droplet size is measured by dynamic light scattering.

In embodiments, the aqueous ultrasound coupling agents provided herein have a shelf life of the aqueous ultrasound coupling agent is at least about 10 hours.

In embodiments, the ratio of the at least one triglyceride to the at least one phospholipid in the aqueous ultrasound coupling agents provided herein is about 16.7:1 by weight.

In embodiments, provided herein is a method of obtaining an ultrasound image of an organ comprising i) contacting the organ with an ultrasound coupling agent provided herein; ii) obtaining an ultrasound image of the organ, wherein the ultrasound coupling agent has an attenuation constant from about 0.15 dB/(MHz*cm) to about 0.60 dB/(MHz*cm) or from about 0.002 to about 1.0 dB/(MHz*cm).

In embodiments, the organ is a brain, breast, colon, or heart. In embodiments, the organ is a brain.

In embodiments, provided herein are aqueous ultrasound coupling agents consisting of: (a) from about 60.0 g/L to about 80.0 g/L of at least one triglyceride; (b) from about 3.2 g/L to about 5.2 g/L of at least one phospholipid; (c) water; (d) pH buffer; and (e) glycerol. In embodiments, provided herein are aqueous ultrasound coupling agents consisting of: (a) at least one triglyceride; (b) at least one phospholipid; (c) water, (d) pH buffer; and (e) glycerol; and wherein the ratio of the at least one triglyceride to the at least one phospholipid is from about 13:1 to about 20:1 by weight. In embodiments, provided herein are aqueous ultrasound coupling agents consisting of: (a) between 5% w/v and 9% w/v of at least one triglyceride; (b) from about 0.2% w/v to about 1.2% w/v of at least one phospholipid; (c) water; (d) pH buffer; and (e) glycerol. In embodiments, provided herein are aqueous ultrasound coupling agents consisting of: (a) between 5% w/v and 9% w/v of at least one triglyceride; (b) at least one phospholipid, wherein the at least one phospholipid is present at about 0.42% w/v; (c) water; (d) pH buffer; and (e) glycerol. In embodiments, provided herein are aqueous ultrasound coupling agents consisting of: (a) between 5% w/v and 9% w/v of at least one triglyceride; (b) at least one phospholipid; (c) water; (d) pH buffer; and (e) glycerol. In embodiments, provided herein are aqueous ultrasound coupling agents consisting of: (a) about 7% w/v of a triglyceride; (b) about 4.2 g/L of a phospholipid; (c) about 23.7 g/L of glycerol; (d) water; and (e) a pH buffer. In embodiments, provided herein are aqueous ultrasound coupling agents comprising: (a) from about 60.0 g/L to about 80.0 g/L of at least one triglyceride; (b) from about 3.2 g/L to about 5.2 g/L of at least one phospholipid; (c) water; (d) pH buffer; and (e) glycerol, wherein the coupling agent does not include a stabilizer and does not include sodium chloride. In embodiments, provided herein are aqueous ultrasound coupling agents comprising: (a) at least one triglyceride; (b) at least one phospholipid; (c) water, (d) pH buffer; and (e) glycerol; and wherein the ratio of the at least one triglyceride to the at least one phospholipid is from about 13:1 to about 20:1 by weight, wherein the coupling agent does not include a stabilizer and does not include sodium chloride. In embodiments, provided herein are aqueous ultrasound coupling agents comprising: (a) between 5% w/v and 9% w/v of at least one triglyceride; (b) from about 0.2% w/v to about 1.2% w/v of at least one phospholipid; (c) water; (d) pH buffer; and (e) glycerol, wherein the coupling agent does not include a stabilizer and does not include sodium chloride. In embodiments, provided herein are aqueous ultrasound coupling agents comprising: (a) between 5% w/v and 9% w/v of at least one triglyceride; (b) at least one phospholipid, wherein the at least one phospholipid is present at about 0.42% w/V; (c) water; (d) pH buffer; and (e) glycerol, wherein the coupling agent does not include a stabilizer and does not include sodium chloride. In embodiments, provided herein are aqueous ultrasound coupling agents comprising: (a) between 5% w/v and 9% w/v of at least one triglyceride; (b) at least one phospholipid; (c) water; (d) pH buffer; and (e) glycerol, wherein the coupling agent does not include a stabilizer and does not include sodium chloride. In embodiments, provided herein are aqueous ultrasound coupling agents comprising: (a) about 7% w/v of a triglyceride; (b) about 4.2 g/L of a phospholipid; (c) about 23.7 g/L of glycerol; (d) water; and (e) a pH buffer, wherein the coupling agent does not include a stabilizer and does not include sodium chloride. In embodiments, provided herein are aqueous ultrasound coupling agents consisting essentially of: (a) from about 60.0 g/L to about 80.0 g/L of at least one triglyceride; (b) from about 3.2 g/L to about 5.2 g/L of at least one phospholipid; (c) water; (d) pH buffer; and (e) glycerol. In embodiments, provided herein are aqueous ultrasound coupling agents consisting essentially of: (a) at least one triglyceride; (b) at least one phospholipid; (c) water, (d) pH buffer; and (e) glycerol; and wherein the ratio of the at least one triglyceride to the at least one phospholipid is from about 13:1 to about 20:1 by weight. In embodiments, provided herein are aqueous ultrasound coupling agents consisting essentially of: (a) between 5% w/v and 9% w/v of at least one triglyceride; (b) from about 0.2% w/v to about 1.2% w/v of at least one phospholipid; (c) water; (d) pH buffer; and (e) glycerol. In embodiments, provided herein are aqueous ultrasound coupling agents consisting essentially of: (a) between 5% w/v and 9% w/v of at least one triglyceride; (b) at least one phospholipid, wherein the at least one phospholipid is present at about 0.42% w/v; (c) water; (d) pH buffer; and (e) glycerol. In embodiments, provided herein are aqueous ultrasound coupling agents consisting essentially of: (a) between 5% w/v and 9% w/v of at least one triglyceride; (b) at least one phospholipid; (c) water; (d) pH buffer; and (e) glycerol. In embodiments, provided herein are aqueous ultrasound coupling agents consisting essentially of: (a) about 7% w/v of a triglyceride; (b) about 4.2 g/L of a phospholipid; (c) about 23.7 g/L of glycerol; (d) water; and (e) a pH buffer. In embodiments, provided herein are aqueous ultrasound coupling agents comprising: (a) about 7% w/v of a triglyceride; (b) about 4.2 g/L of at least one phospholipid; (c) about 23.7 g/L of glycerol; (d) water; and (e) a pH buffer. In embodiments, the stabilizer is polysorbate 80 (PS80).

In embodiments, provided herein are aqueous ultrasound coupling agents consisting of: (a) from about 60.0 g/L to about 80.0 g/L of at least one triglyceride; (b) from about 3.2 g/L to about 5.2 g/L of lecithin; (c) water; (d) pH buffer; and (e) glycerol. In embodiments, provided herein are aqueous ultrasound coupling agents consisting of: (a) at least one triglyceride; (b) lecithin; (c) water, (d) pH buffer; and (e) glycerol; and wherein the ratio of the at least one triglyceride to the lecithin is from about 13:1 to about 20:1 by weight. In embodiments, provided herein are aqueous ultrasound coupling agents consisting of: (a) between 5% w/v and 9% w/v of at least one triglyceride; (b) from about 0.2% w/v to about 1.2% w/v of lecithin; (c) water; (d) pH buffer; and (e) glycerol. In embodiments, provided herein are aqueous ultrasound coupling agents consisting of: (a) between 5% w/v and 9% w/v of at least one triglyceride; (b) lecithin, wherein the lecithin is present at about 0.42% w/v; (c) water; (d) pH buffer; and (e) glycerol. In embodiments, provided herein are aqueous ultrasound coupling agents consisting of: (a) between 5% w/v and 9% w/v of at least one triglyceride; (b) lecithin; (c) water; (d) pH buffer; and (e) glycerol. In embodiments, provided herein are aqueous ultrasound coupling agents consisting of: (a) about 7% w/v of a triglyceride; (b) about 4.2 g/L of lecithin; (c) about 23.7 g/L of glycerol; (d) water; and (e) a pH buffer. In embodiments, provided herein are aqueous ultrasound coupling agents comprising: (a) from about 60.0 g/L to about 80.0 g/L of at least one triglyceride; (b) from about 3.2 g/L to about 5.2 g/L of lecithin; (c) water; (d) pH buffer; and (e) glycerol, wherein the coupling agent does not include a stabilizer and does not include sodium chloride. In embodiments, provided herein are aqueous ultrasound coupling agents comprising: (a) at least one triglyceride; (b) lecithin; (c) water, (d) pH buffer; and (e) glycerol; and wherein the ratio of the at least one triglyceride to the lecithin is from about 13:1 to about 20:1 by weight, wherein the coupling agent does not include a stabilizer and does not include sodium chloride. In embodiments, provided herein are aqueous ultrasound coupling agents comprising: (a) between 5% w/v and 9% w/v of at least one triglyceride; (b) from about 0.2% w/v to about 1.2% w/v of lecithin; (c) water; (d) pH buffer; and (e) glycerol, wherein the coupling agent does not include a stabilizer and does not include sodium chloride. In embodiments, provided herein are aqueous ultrasound coupling agents comprising: (a) between 5% w/v and 9% w/v of at least one triglyceride; (b) lecithin, wherein the lecithin is present at about 0.42% w/v; (c) water; (d) pH buffer; and (e) glycerol, wherein the coupling agent does not include a stabilizer and does not include sodium chloride. In embodiments, provided herein are aqueous ultrasound coupling agents comprising: (a) between 5% w/v and 9% w/v of at least one triglyceride; (b) lecithin; (c) water; (d) pH buffer; and (e) glycerol, wherein the coupling agent does not include a stabilizer and does not include sodium chloride. In embodiments, provided herein are aqueous ultrasound coupling agents comprising: (a) about 7% w/v of a triglyceride; (b) about 4.2 g/L of lecithin; (c) about 23.7 g/L of glycerol; (d) water; and (e) a pH buffer, wherein the coupling agent does not include a stabilizer and does not include sodium chloride. In embodiments, provided herein are aqueous ultrasound coupling agents consisting essentially of: (a) from about 60.0 g/L to about 80.0 g/L of at least one triglyceride; (b) from about 3.2 g/L to about 5.2 g/L of lecithin; (c) water; (d) pH buffer; and (e) glycerol. In embodiments, provided herein are aqueous ultrasound coupling agents consisting essentially of: (a) at least one triglyceride; (b) lecithin; (c) water, (d) pH buffer; and (e) glycerol; and wherein the ratio of the at least one triglyceride to the lecithin is from about 13:1 to about 20:1 by weight. In embodiments, provided herein are aqueous ultrasound coupling agents consisting essentially of: (a) between 5% w/v and 9% w/v of at least one triglyceride; (b) from about 0.2% w/v to about 1.2% w/v of lecithin; (c) water; (d) pH buffer; and (e) glycerol. In embodiments, provided herein are aqueous ultrasound coupling agents consisting essentially of: (a) between 5% w/v and 9% w/v of at least one triglyceride; (b) lecithin, wherein the lecithin is present at about 0.42% w/v; (c) water; (d) pH buffer; and (e) glycerol. In embodiments, provided herein are aqueous ultrasound coupling agents consisting essentially of: (a) between 5% w/v and 9% w/v of at least one triglyceride; (b) lecithin; (c) water; (d) pH buffer; and (e) glycerol. In embodiments, provided herein are aqueous ultrasound coupling agents consisting essentially of: (a) about 7% w/v of a triglyceride; (b) about 4.2 g/L of lecithin; (c) about 23.7 g/L of glycerol; (d) water; and (e) a pH buffer. In embodiments, provided herein are aqueous ultrasound coupling agents comprising: (a) about 7% w/v of a triglyceride; (b) about 4.2 g/L of lecithin; (c) about 23.7 g/L of glycerol; (d) water; and (e) a pH buffer. In embodiments, the stabilizer is polysorbate 80 (PS80). In embodiments, the ultrasound coupling agent has an attenuation coefficient of from about 0.15 dB/(MHz*cm) to about 0.60 dB/(MHz*cm). coupling agent has an attenuation coefficient of from about 0.002 dB/(MHz*cm) to about 1.0 dB/(MHz*cm). In embodiments, the aqueous ultrasound coupling agent has an attenuation coefficient of from about 0.05 dB/(MHz*cm) to about 15 dB/(MHz*cm).

In embodiments, provided herein are methods of obtaining an ultrasound image of an organ or a cavity comprising i) contacting the organ or the cavity with an ultrasound coupling agent described herein; and ii) obtaining an ultra sound image of the organ or the cavity.

In embodiments, provided herein are containers comprising aqueous ultrasound coupling agents described herein.

In embodiments, provided herein are kits comprising an aqueous ultrasound coupling agent described herein and a container. In embodiments, the container comprises the ultrasound coupling agent. In embodiments, the kit contains a bag spike. In embodiments, the kit comprises a syringe. In embodiments, the kit comprises a filter.

In embodiments, provided herein are methods of performing neurosurgery comprising contacting an instrument used for surgery with an aqueous ultrasound coupling agent described herein and inserting the instrument into a brain.

### BRIEF DESCRIPTION OF THE DRAWINGS

FIGS. 1A-1G show ultrasound images of a brain tissue-mimicking phantom filled with saline (FIG. 1A), Formulation 2 (FIG. 1B), Formulation 3 (FIG. 1C), Formulation 4 (FIG. 1D), Formulation 5 (FIG. 1E), Formulation 6 (FIG. 1F), and Formulation 7 (FIG. 1G). The components of each of Formulations 1-7 are provided in Example 2.

FIGS. 2A-2B show ultrasound images of a cavity filled containing a tumor. The cavity is filled with saline (FIG. 2A) or the aqueous ultrasound coupling agent, Formulation 7 (FIG. 2B). The components of Formulation 7 are provided in Example 2.

FIG. 3 shows an exemplary kit of the disclosure containing a syringe, a bag spike, and an aqueous ultrasound coupling agent.

### DETAILED DESCRIPTION

### A. Definitions

As used herein, the term “ultrasound coupling agent” refers to a medium used to exclude air between the ultrasound probe and the tissue to be imaged. The main purpose of the ultrasound coupling agent is thus to provide acoustic coupling between the ultrasound transducer/probe and the tissue to be investigated. The term is synonymous with terms like “contact fluid”, “contact agent”, “coupling agent”, “coupling fluid”, “acoustic fluid”, “acoustic coupling fluid” and “acoustic coupling agent”. In one embodiment, the ultrasound coupling agent described herein is in the form of a liquid or gel under standard conditions of temperature and pressure and/or at physiological temperatures and ambient pressure. Typically the ultrasound coupling agent is in the form of an emulsion.

The term “aqueous” as used herein refers to a solution or mixture comprising water as the solvent or continuous phase. This includes cases where an aqueous phase can diffuse freely within the confines of a free-standing three-dimensional structure such as a hydrogel or an emulsion-based hydrogel (e.g. an emulgel). The term does not exclude solvents other than water from being present in the solution/mixture, however typically water is the principal solvent present (e.g., forming at least 50% by weight of the solvent present in the solution/mixture). In a preferred embodiment, the term “aqueous” refers to a solution or mixture comprising water as the solvent or continuous phase, and in which water forms at least 90% by weight of the solvent (or continuous phase) present in the solution/mixture. In one embodiment, water is the only solvent present in the solution/mixture (or continuous phase). Aqueous solutions or mixtures may contain salts such as sodium and/or potassium salts and may, for example be isotonic. Aqueous solutions or mixtures may be sterile, as appropriate.

In embodiments, the aqueous ultrasound coupling agent is for use in invasive ultrasound imaging of a subject. As used herein, the term “invasive ultrasound imaging” refers to any method for imaging of a body, particularly the human body, which involves the detection of ultrasound and which involves the introduction of a device (such as the tip of an ultrasonic probe/transducer) into the body either through a body orifice or through an opening in the body other than a body orifice, such as a break or rupture in the skin. The term “body orifice” means any natural opening in the body, as well as the external surface of the eyeball, or any permanent artificial opening, such as a stoma. Invasive ultrasound imaging thus includes both the imaging of a body via the introduction of a device through a natural opening in the body and the imaging of a body via the introduction of a device through an artificial opening, such an incision made during surgery or as part of an invasive diagnostic procedure.

As used herein, the term “ultrasound” refers to sound waves with frequencies higher than the upper audible limit of human hearing. Typically, this is about 20 KHz and so in general ultrasound waves have a frequency greater than 20 kHz. In the applications described herein, however, frequencies in the range of about 5 to about 20 MHz are contemplated. Preferably therefore, the term “ultrasound imaging” as used herein refers to any method for imaging involving the detection of sound waves having frequencies in the range of 5 to 20 MHz. “Imaging” may involve the display of a static and/or moving visual image, such as on paper or a display screen, or may involve a computer or artificial intelligence device generating and/or processing data that could be rendered as a visual image, whether or not such an image is in fact generated.

As used herein, amounts which are indicated as “about”, “approximately”, etc may be the exact value stated or may vary by +10%, preferably +5% or +1%. The same applies to the ends of ranges. Formulations “comprising” certain components may also “consist essentially” of such components or consist solely of such components. Similarly, compositions “consisting essentially of” listed components may consist solely of such components or may contain 105<preferably 5% or 1% of other materials. All % are by weight where context allows, unless indicated otherwise.

### B. Aqueous Ultrasound Coupling Agents

Provided herein are ultrasound coupling agents and methods of using them in ultrasound imaging of a subject. In embodiments, the ultrasound imaging is invasive ultrasound imaging. The ultrasound coupling agents described herein are unexpectedly effective in reducing the appearance of artifacts such as brightening artifacts when used in the invasive ultrasound imaging of body tissues, particularly human body tissues, compared to saline, and also safe. Advantageously, the ultrasound coupling agent does not contain sodium chloride or a stabilizing agent like polysorbate 80 (also known as PS80 or tween 80). Prior to the present disclosure, it was believed that sodium chloride and polysorbate 80 were needed to produce a stable and effective ultrasound coupling agent. Sodium chloride was selected to maintain osmolality of the solution. However, sodium chloride disrupts fat and an emulsifier (PS80) must be used to stabilize the solution, by preventing fat separation. The prior formulation thus included triglyceride, phospholipid, NaCl and PS80, while providing an attenuation constant appropriate for providing an ultrasound image with minimal artifacts. However, applicant discovered the prior formulations were not safe for ultrasound imaging of the brain. Advantageously, applicant discovered that an effective aqueous ultrasound coupling agent can be prepared without polysorbate 80 and sodium chloride. The aqueous ultrasound coupling agents described herein that lacked PS80 and sodium chloride were superior to or substantially similar to formulations that contained PS80 and sodium chloride for ultrasound imaging of the brain. (Example 2).

In embodiments, provided herein are aqueous ultrasound coupling agents consisting of: (a) from about 60.0 g/L to about 80.0 g/L of at least one triglyceride; (b) from about 3.2 g/L to about 5.2 g/L of at least one phospholipid; (c) water; (d) pH buffer; and (e) glycerol. In embodiments, provided herein are aqueous ultrasound coupling agents consisting of: (a) at least one triglyceride; (b) at least one phospholipid; (c) water, (d) pH buffer; and (e) glycerol; and wherein the ratio of the at least one triglyceride to the at least one phospholipid is from about 13:1 to about 20:1 by weight. In embodiments, provided herein are aqueous ultrasound coupling agents consisting of: (a) between 5% w/v and 9% w/v of at least one triglyceride; (b) from about 0.2% w/v to about 1.2% w/v of at least one phospholipid; (c) water; (d) pH buffer; and (e) glycerol. In embodiments, provided herein are aqueous ultrasound coupling agents consisting of: (a) between 5% w/v and 9% w/v of at least one triglyceride; (b) at least one phospholipid, wherein the at least one phospholipid is present at about 0.42% w/v; (c) water; (d) pH buffer; and (e) glycerol. In embodiments, provided herein are aqueous ultrasound coupling agents consisting of: (a) between 5% w/v and 9% w/v of at least one triglyceride; (b) at least one phospholipid; (c) water; (d) pH buffer; and (e) glycerol. In embodiments, provided herein are aqueous ultrasound coupling agents consisting of: (a) between 5% w/v and 9% w/v of at least one triglyceride; (b) from about 0.2% w/v to about 1.2% w/v of at least one phospholipid; (c) water; (d) pH buffer; and (e) from about 21 g/L to about 26 g/L of glycerol. In embodiments, provided herein are aqueous ultrasound coupling agents consisting of: (a) about 7% w/v of a triglyceride; (b) about 4.2 g/L of a phospholipid; (c) about 23.7 g/L of glycerol; (d) water; and (e) a pH buffer. In embodiments, the pH buffer is NaOH.

In embodiments, provided herein are aqueous ultrasound coupling agents consisting of: (a) from about 60.0 to about 80.0 g/L of at least one triglyceride; (b) from about 3.2 to about 5.2 g/L of lecithin; (c) water; (d) pH buffer; and (e) glycerol. In embodiments, provided herein are aqueous ultrasound coupling agents consisting of: (a) at least one triglyceride; (b) lecithin; (c) water, (d) pH buffer; and (e) glycerol; and wherein the ratio of the at least one triglyceride to the at least one phospholipid is from about 13:1 to about 20:1 by weight. In embodiments, provided herein are aqueous ultrasound coupling agents consisting of: (a) between 5% w/v and 9% w/v of at least one triglyceride; (b) from about 0.2% w/v to about 1.2% w/v of lecithin; (c) water; (d) pH buffer; and (e) glycerol. In embodiments, provided herein are aqueous ultrasound coupling agents consisting of: (a) between 5% w/v and 9% w/v of at least one triglyceride; (b) about 0.42% w/v lecithin; (c) water; (d) pH buffer; and (e) glycerol. In embodiments, provided herein are aqueous ultrasound coupling agents consisting of: (a) between 5% w/v and 9% w/v of at least one triglyceride; (b) lecithin; (c) water; (d) pH buffer; and (e) glycerol. In embodiments, provided herein are aqueous ultrasound coupling agents consisting of: (a) between 5% w/v and 9% w/v of at least one triglyceride; (b) from about 0.2% w/v to about 1.2% w/v of lecithin; (c) water; (d) pH buffer; and (e) from about 21 g/L to about 26 g/L of glycerol. In embodiments, provided herein are aqueous ultrasound coupling agents consisting of: (a) 7% w/v of a triglyceride; (b) about 4.2 g/L lecithin; (c) about 23.7 g/L of glycerol; (d) water; and (e) a pH buffer. In embodiments, the pH buffer is NaOH.

In embodiments, provided herein are aqueous ultrasound coupling agents comprising: (a) from about 60.0 to about 80.0 g/L of at least one triglyceride; (b) from about 3.2 to about 5.2 g/L of at least one phospholipid; (c) water; (d) pH buffer; and (e) glycerol; wherein the coupling agent does not include a stabilizer and does not include sodium chloride. In embodiments, provided herein are aqueous ultrasound coupling agents comprising: (a) at least one triglyceride; (b) at least one phospholipid; (c) water; (d) pH buffer; and (e) glycerol; and wherein the ratio of the at least one triglyceride to the at least one phospholipid is from about 13:1 to about 20:1 by weight; wherein the coupling agent does not include a stabilizer and does not include sodium chloride. In embodiments, provided herein are aqueous ultrasound coupling agents comprising: (a) between 5% w/v and 9% w/v of at least one triglyceride; (b) from about 0.2% w/v to about 1.2% w/v of at least one phospholipid; (c) water; (d) pH buffer; and (e) glycerol; wherein the coupling agent does not include a stabilizer and does not include sodium chloride. In embodiments, provided herein are aqueous ultrasound coupling agents comprising: (a) between 5% w/v and 9% w/v of at least one triglyceride; (b) at least one phospholipid, wherein the at least one phospholipid is present at about 0.42% w/v; (c) water; (d) pH buffer; and (e) glycerol; wherein the coupling agent does not include a stabilizer and does not include sodium chloride. In embodiments, provided herein are aqueous ultrasound coupling agents comprising: (a) between 5% w/v and 9% w/v of at least one triglyceride; (b) at least one phospholipid; (c) water; (d) pH buffer; and (e) glycerol; wherein the coupling agent does not include a stabilizer and does not include sodium chloride. In embodiments, provided herein are aqueous ultrasound coupling agents comprising: (a) between 5% w/v and 9% w/v of at least one triglyceride; (b) from about 0.2% w/v to about 1.2% w/v of at least one phospholipid; (c) water; (d) pH buffer; and (e) from about 21 g/L to about 26 g/L of glycerol; wherein the coupling agent does not include a stabilizer and does not include sodium chloride. In embodiments, provided herein are aqueous ultrasound coupling agents comprising: (a) about 7% w/v of a triglyceride; (b) about 4.2 g/L of a phospholipid; (c) about 23.7 g/L of glycerol; (d) water; and (e) a pH buffer; wherein the coupling agent does not include a stabilizer and does not include sodium chloride. In embodiments, the pH buffer is NaOH. In embodiments, the stabilizer is PS80.

In embodiments, provided herein are aqueous ultrasound coupling agents comprising: (a) from about 60.0 to about 80.0 g/L of at least one triglyceride; (b) from about 3.2 to about 5.2 g/L of lecithin; (c) water; (d) pH buffer; and (e) glycerol; wherein the coupling agent does not include a stabilizer and does not include sodium chloride. In embodiments, provided herein are aqueous ultrasound coupling agents comprising: (a) at least one triglyceride; (b) lecithin; (c) water; (d) pH buffer; and (e) glycerol; and wherein the ratio of the at least one triglyceride to the at least one phospholipid is from about 13:1 to about 20:1 by weight; wherein the coupling agent does not include a stabilizer and does not include sodium chloride. In embodiments, provided herein are aqueous ultrasound coupling agents comprising: (a) between 5% w/v and 9% w/v of at least one triglyceride; (b) from about 0.2% w/v to about 1.2% w/v of lecithin; (c) water; (d) pH buffer; and (e) glycerol; wherein the coupling agent does not include a stabilizer and does not include sodium chloride. In embodiments, provided herein are aqueous ultrasound coupling agents comprising: (a) between 5% w/v and 9% w/v of at least one triglyceride; (b) about 0.42% w/v lecithin; (c) water; (d) pH buffer; and (e) glycerol; wherein the coupling agent does not include a stabilizer and does not include sodium chloride. In embodiments, provided herein are aqueous ultrasound coupling agents comprising: (a) between 5% w/v and 9% w/v of at least one triglyceride; (b) lecithin; (c) water; (d) pH buffer; and (e) glycerol; wherein the coupling agent does not include a stabilizer and does not include sodium chloride. In embodiments, provided herein are aqueous ultrasound coupling agents comprising: (a) between 5% w/v and 9% w/v of triglyceride; (b) from about 0.2% w/v to about 1.2% w/v of at least one lecithin; (c) water; (d) pH buffer; and (e) from about 21 g/L to about 26 g/L of glycerol; wherein the coupling agent does not include a stabilizer and does not include sodium chloride. In embodiments, provided herein are aqueous ultrasound coupling agents comprising: (a) about 7% w/v of a triglyceride; (b) about 4.2 g/L lecithin; (c) about 23.7 g/L of glycerol; (d) water; and (e) a pH buffer; wherein the coupling agent does not include a stabilizer and does not include sodium chloride. In embodiments, the pH buffer is NaOH. In embodiments, the stabilizer is PS80.

In embodiments, provided herein are aqueous ultrasound coupling agents comprising: (a) about 7% w/v of a triglyceride; (b) about 4.2 g/L of at least one phospholipid; (c) about 23.7 g/L of glycerol; (d) water; and (e) a pH buffer. In embodiments, provided herein are aqueous ultrasound coupling agents comprising (a) about 7% w/v of a triglyceride; (b) about 4.2 g/L lecithin; (c) about 23.7 g/L of glycerol; (d) water; and (e) a pH buffer. In embodiments, the pH buffer is NaOH.

### Triglycerides

In embodiments, the aqueous ultrasound coupling agent comprises 20.0 to 90.0 g/L of at least one triglyceride. As used herein, the term “triglyceride” (sometimes also “triacylglycerol” or “triacylglyceride”) refers to a neutral tri-ester derived from glycerol and three fatty acids. The triglyceride as defined herein therefore contains a polar glycerol-derived “head group” and three non-polar fatty-acid derived “tail groups”. The three non-polar tail groups may have the same or a differing number of carbon atoms and may each independently be saturated or unsaturated. It is preferred however that at least one of the three non-polar tail groups is unsaturated.

Examples of suitable non-polar groups include C6-C₃₂ alkyl and alkenyl groups, which are typically present as the esters of long chain carboxylic acids. These are often described by reference to the number of carbon atoms and the number of unsaturations in the carbon chain. Thus, CX: Z indicates a hydrocarbon chain having X carbon atoms and Z unsaturations. Examples particularly include lauroyl (C12:0), myristoyl (C14:0), palmitoyl (C16:0), phytanoyl (C16:0), palmitoleoyl (C16:1), stearoyl (C18:0), oleoyl (C18:1), elaidoyl (C18:1), linoleoyl (C18:2), linolenoyl (C18:3), arachidonoyl (C20:4), behenoyl (C22:0) and lignoceroyl (C24:9) groups. Thus, typical non-polar chains are based on the fatty acids of natural ester lipids, including caproic, caprylic, capric, lauric, myristic, palmitic, phytanic, palmitolic, stearic, oleic, elaidic, linoleic, linolenic, arachidonic, behenic or lignoceric acids, or the corresponding alcohols. Preferable non-polar chains are palmitic, stearic, oleic and linoleic acids, particularly linoleic acid. In one preferred embodiment, component a) comprises at least one triglyceride having one or more C16 to C18 alkyl groups, particularly such groups having zero, one or two unsaturations. In particular, component a) may comprise at least 50% of triglycerides having such alkyl groups.

The at least one triglyceride used as part of component a) may be synthetic or may be derived from a purified and/or chemically modified natural source. In one particularly preferred embodiment, the at least one triglyceride is derived from a natural source. For example, the triglyceride used may be selected from vegetable oils or animal fats. Vegetable oils may preferably be selected from the group consisting of soybean oil, olive oil, palm oil and copra oil. Examples of animal fats that may be used are milk fats, fish oils and fish liver oils. The use of soybean oil (which is typically a mixture of neutral triglycerides) is especially preferred.

In embodiments, the triglyceride is present in the ultrasound coupling agent in an amount of 20.0 to 90.0 g/L (e.g., 20, 25, 30, 35, 40, 45, 50, 55, 60, 65, 70, 75, 80, 85, or 90 g/L). In embodiments, the triglyceride is present in an amount of 40.0 to 90.0 g/L, 50.0 to 90.0 g/L, 55.0 to 85.0 g/L, or 60.0 to 80.0 g/L. Where a mixture of triglycerides is used, the amount (g/L) of triglyceride refers to the sum of the amount of each of the triglycerides in the composition as a whole.

When using triglycerides obtained from commercial sources, particular natural products such as soybean oil, there is generally a certain proportion of “impurity” lipid having other chain lengths etc. In embodiments, the compositions and methods described herein encompass such “impurities,” and the use of “consistent of” or “consisting essentially of” does not exclude the presence of such “impurities.” In embodiments, a triglyceride may be any pharmaceutically acceptable grade of triglyceride with concomitant impurities (i.e. a triglyceride of commercial purity). These impurities may be separated and removed by purification but providing the grade is consistent this is rarely necessary. If necessary, however, a triglyceride may be essentially chemically pure triglyceride, such as at least 80% pure, at least 85% pure, at least 90% pure (e.g., 90%, 91%, 92%, 93%, 94%, 95%, 96%, 97%, 98%, 99% or more) triglyceride.

Given that the aqueous ultrasound coupling agent is intended for use on the body, it will be readily appreciated by the skilled person in the art that certain additional requirements must also typically be satisfied by the at least one triglyceride. In particular, the at least one triglyceride and its component fatty acids must generally be non-toxic, sterile and biocompatible. In a preferred embodiment therefore, the at least one triglyceride is at least one pharmaceutical grade triglyceride.

In embodiments, the at least one triglyceride is from one or more of olive oil, coconut oil, palm oil, canola oil, soybean oil, sunflower oil, corn oil, peanut oil, safflower oil, cottonseed oil, sesame oil, flaxseed oil, walnut oil, avocado oil, rice bran oil, grapeseed oil, almond oil, hazelnut oil, macadamia nut oil, pumpkin seed oil, hemp seed oil, camelina oil, mustard oil, pistachio oil, pecan oil, poppy seed oil, black seed oil, apricot kernel oil, cherry kernel oil, peach kernel oil, tamanu oil, borage oil, evening primrose oil, sea buckthorn oil, perilla oil, moringa oil, neem oil, karanja oil, jojoba oil, tung oil, camellia oil, tea seed oil, babassu oil, murumuru oil, safflower oil, shea butter, cocoa butter, sal butter, kokum butter, mango butter, illipe butter, or fish oil. In embodiments, the at least one triglyceride is from soybean oil.

In embodiments, the one or more triglycerides are selected from any one of: tristearin, triolein, tripalmitin, triacetin, trilinolein, tricaprin, trilaurin, triarachidin, tridecanoin, trilinolenin, tricaprylin, tridecanoin, trimyristin, tricaprylin, trivalerate, tricaprate, trilinolenin, tridecanoate, triundecanoin, and trinonanoin.

In embodiments, the one or more triglycerides have a structure of Formula I:

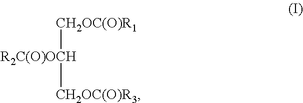

wherein, R1, R2, and R3 are each independently a C6-C32 saturated or unsaturated alkyl.

In embodiments, R₁C(O), R₂C(O), and R₃C(O) of Formula I each independently comprise a fatty acid residue. In embodiments, the at least one triglycerides of the aqueous ultrasound coupling agents comprise a residue of linoleic acid (44-62% by weight), oleic acid (19-30% by weight), palmitic acid (7-14% by weight), α-linolenic acid (4-11% by weight), and stearic acid (1.4-5.5% by weight). In embodiments, the aqueous ultrasound coupling agents described herein comprise one or more triglycerides selected from any one of: linolenic-oleic-linolenic, linoleic-linoleic-linoleic, oleic-linoleic-oleic, linoleic-oleic-linoleic, linolenic-linoleic-linolenic, palmitic-oleic-linoleic, palmitic-linoleic-palmitic, stearic-oleic-linoleic, linoleic-linolenic-linoleic, and linoleic-oleic-oleic.

In embodiments, the at least one triglyceride is a medium-chain triglyceride. A medium chain triglyceride comprises a glycerol backbone and three fatty acid chains that are 6-12 carbons long.

In embodiments, the one or more triglycerides comprise linoleic acid, oleic acid, palmitic acid, α-linolenic acid, and stearic acid.

In embodiments, the at least one triglyceride is present in the aqueous ultrasound coupling agent at a concentration of from about 1% w/v and 15% w/v. In embodiments, the at least one triglyceride is present in the aqueous ultrasound coupling agent at a concentration of about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6.0% w/v, about 6.5% w/v, about 7.0% w/v, about 7.5% w/v, about 8.0% w/v, about 8.5% w/v, about 9% w/v, about 9.5% w/v, about 10% w/v, about 10.5% w/v, about 11% w/v, about 11.5% w/v, about 12% w/v, about 12.5% w/v, about 13% w/v, about 13.5% w/v, about 14% w/v, about 14.5% w/v, about 15% w/v, or any value or range therebetween.

In embodiments, the at least one triglyceride is present in the aqueous ultrasound coupling agent at a concentration of between 5% w/v and 9% w/v. In embodiments, the at least one triglyceride is present in the aqueous ultrasound coupling agent at a concentration of 5.1% w/v, 5.2% w/v, 5.3% w/v, 5.4% w/v, 5.5% w/v, 5.6% w/v, 5.7% w/v, 5.8% w/v, 5.9% w/v, 6.0% w/v, 6.1% w/v, 6.2% w/v, 6.3% w/v, 6.4% w/v, 6.5% w/v, 6.6% w/v, 6.7% w/v, 6.8% w/v, 6.9% w/v, 7.0% w/v, 7.1% w/v, 7.2% w/v, 7.3% w/v, 7.4% w/v, 7.5% w/v, 7.6% w/v, 7.7% w/v, 7.8% w/v, 7.9% w/v, 8.0% w/v, 8.1% w/v, 8.2% w/v, 8.3% w/v, 8.4% w/v, 8.5% w/v, 8.6% w/v, 8.7% w/v, 8.8% w/v, 8.9% w/v, or any value or range therebetween. In embodiments, the at least one triglyceride is present in the aqueous ultrasound coupling agent at a concentration of about 7% w/v.

In embodiments, the at least one triglyceride is present in the aqueous ultrasound coupling agent at a concentration of between 50 g/L and 90 g/L. In embodiments, the at least one triglyceride is present in the aqueous ultrasound coupling agent at a concentration of about 50 g/L, about 51 g/L, about 52 g/L, about 53 g/L, about 54 g/L, about 55 g/L, about 56 g/L, about 57 g/L, about 58 g/L, about 59 g/L, about 60 g/L, about 61 g/L, about 62 g/L, about 63 g/L, about 64 g/L, about 65 g/L, about 66 g/L, about 67 g/L, about 68 g/L, about 69 g/L, about 70 g/L, about 71 g/L, about 72 g/L, about 73 g/L, about 74 g/L, about 75 g/L, about 76 g/L, about 77 g/L, about 78 g/L, about 79 g/L, about 80 g/L, about 81 g/L, about 82 g/L, about 83 g/L, about 84 g/L, about 85 g/L, about 86 g/L, about 87 g/L, about 88 g/L, about 89 g/L, about 90 g/L, or any concentration or range therebetween. In embodiments, the at least one triglyceride is present in the aqueous ultrasound coupling agent at a concentration of about 70 g/L.

### Phospholipids or Lecithin

Phospholipid comprises a polar head group and at least one non-polar tail group. The difference between components triglycerides and phospholipid lies principally in the polar group. The non-polar portions may be derived from the fatty acids or corresponding alcohols considered above for the triglycerides. In particular C16 to C18 acyl groups having zero, one or two unsaturations are suitable as moieties forming the non-polar group of the phospholipids. It will typically be the case that the phospholipid will contain two non-polar groups, although one or more constituents of this component may have only one non-polar moiety. Where more than one non-polar group is present these may be the same or different.

The at least one phospholipid comprises a negatively charged phosphate group. The phospholipid is thus an ionic species. Preferred phospholipid polar “head” groups include phosphatidylcholine (PC), phosphatidylethanolamine (PE), phosphatidylserine (PS) and phosphatidylinositol (PI). PC and PE are preferred lipids, both individually and as a mixture. In one embodiment, component b) may comprise at least 70% PC, PE or mixtures thereof. In embodiments, the phospholipid is phosphatidylcholine (PC). In a preferred embodiment, component b) thus comprises at least 50% PC, preferably at least 70% PC and most preferably at least 80% PC.

The phospholipid may be synthetic or derived from a natural source. In an embodiment, the phospholipid is derived from a natural source. Suitable natural sources of phospholipids include egg, heart (e.g. bovine), brain, liver (e.g. bovine) and plant sources including soybean. Such sources may may comprise any mixture of phospholipids. In an embodiment, the phospholipid is lecithin, preferably egg yolk lecithin, soy lecithin or a mixture thereof. Egg yolk lecithin is preferred.

When using phospholipids from natural sources, there is generally a certain proportion of “impurity” lipid or other component, such as triglycerides, tocoopherols, sterols, phosphatides, metals, pigments, and protein fragments. In embodiments, the compositions and methods described herein encompass such “impurities,” and the use of “consistent of” or “consisting essentially of” does not exclude the presence of such “impurities.” In embodiments, a phospholipid may be any pharmaceutically acceptable grade of phospholipid with concomitant impurities (i.e. a triglyceride of commercial purity). These impurities may be separated and removed by purification but providing the grade is consistent this is rarely necessary. If necessary, however, a triglyceride may be essentially chemically pure triglyceride, such as at least 80% pure, at least 85% pure, at least 90% pure, at least 95% pure, (e.g., 90%, 91%, 92%, 93%, 94%, 95%, 96%, 97%, 98%, 99% or more) triglyceride.

In embodiments, the at least one phospholipid is non-toxic, biocompatible and sterile. In embodiments, the at least one phospholipid is any pharmaceutically acceptable phospholipid.

In embodiments, the aqueous ultrasound coupling agents described herein comprise from about 1 g/L to about 12 g/L phospholipid. In embodiments, the aqueous ultrasound coupling agents described herein comprise from about 4.2 g/L to about 12 g/L phospholipid. In embodiments, the aqueous ultrasound coupling agents described herein comprise from about 3.2 g/L to about 5.2 g/L phospholipid. In embodiments, the aqueous ultrasound coupling agents described herein comprise up to about 12 g/L phospholipid. In embodiments, the aqueous ultrasound coupling agents described herein comprise from about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L phospholipid, or any concentration or subrange thereof. In embodiments, the ultrasound coupling agent comprises about 4.2 g/L phospholipid.

In embodiments, the phospholipid is present in an amount of 2.5 to 5.2 g/L of the aqueous ultrasound coupling agent, e.g. 3.0 to 5.0 g/L. In an embodiment, component the phospholipid is present in an amount of 3.5 to 4.8 g/L, 3.8 to 4.5 g/L. Where the coupling agent comprises a mixture of phospholipids, the amount of phospholipid refers to the sum of the amount of each of the phospholipids in the composition as a whole. The coupling agent may contain low levels of impurities (i.e. lipids other than phospholipids) or if desired may be substantially pure.

In one embodiment, the ratio by weight of triglycerides to phospholipids in the aqueous ultrasound coupling agent is in the range of 8:1 to 25:1, preferably 10:1 to 22:1, more preferably 12:1 to 20:1, most preferably 14:1 to 18:1. In embodiments, the ratio of triglycerides to phospholipids by weight ranges from about 13:1 to about 20:1. In embodiments, the ratio of triglycerides to phospholipids by weight is about 16.7:1.

In embodiments, the aqueous ultrasound coupling agent comprises at least one phospholipid in combination with at least one triglyceride.

In embodiments, the aqueous ultrasound coupling agents provided herein comprises lecithin comprising at least one phospholipid (e.g., phosphatidylcholine, phosphatidylethanolamine, phosphatidylinositol). In embodiments, the lecithin further comprises glycerol, choline, glycolipids, triglycerides. In embodiments, the lecithin comprises from about 20% to about 70% phospholipids by weight. In embodiments, the lecithin comprises from about 60% to about 70% phospholipids by weight. In embodiments, the lecithin comprises about 20%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% phospholipids by weight of the lecithin, including all ranges and values therebetween.

In embodiments, the lecithin is from one or more of soybeans, sunflower seeds, rapeseeds, egg yolks, wheat germ, corn, peanuts, sesame seeds, rice bran, flaxseeds, pumpkin seeds, cashews, almonds, hazelnuts, pistachios, walnuts, pecans, macadamia nuts, brazil nuts, chia seeds, hemp seeds, poppy seeds, quinoa, oats, barley, rye, millet, sorghum, buckwheat, amaranth, avocado, olives, cocoa beans, coconut, milk, butter, cheese, yogurt, beef liver, chicken liver, fish roe, mackerel, salmon, sardines, herring, anchovies, trout, halibut, tuna, and egg whites. In embodiments, the lecithin is from egg yolk.

In embodiments, the lecithin comprises phosphatidylcholine and/or phosphatidylethanolamine and/or lysophosphatidyl choline.

In embodiments, the lecithin comprises phosphatidylcholine. In embodiments, the lecithin comprises from about 10% to about 100% phosphatidylcholine by weight. In embodiments, the lecithin comprises from about 10% to about 90% phosphatidylcholine by weight. In embodiments, the lecithin comprises from about 10% to about 80% phosphatidylcholine by weight. In embodiments, the lecithin comprises from about 10% to about 70% phosphatidylcholine by weight. In embodiments, the lecithin comprises from about 20% to about 70% phosphatidylcholine by weight. In embodiments, the lecithin comprises from about 60% to about 70% phosphatidylcholine by weight. In embodiments, the lecithin comprises about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% phosphatidylcholine by weight of the lecithin, including all ranges and values therebetween.

In embodiments, the lecithin comprises s phosphatidylethanolamine. In embodiments, the lecithin comprises from about 5% to about 30% phosphatidylethanolamine by weight. In embodiments, the lecithin comprises about 5%, about 6%, about 7%, about 8%, about 9%, about 10%, about 11%, about 12%, about 13%, about 14%, about 15%, about 16%, about 17%, about 18%, about 19%, about 20%, about 20%, about 21%, about 22%, about 23%, about 24%, about 25%, about 26%, about 27%, about 28%, about 29%, about 30% phosphatidylethanolamine by weight of the lecithin, including all ranges and values therebetween. In embodiments, the lecithin comprises from about 7% to about 10% phosphatidylethanolamine. In embodiments, the lecithin comprises from about 10% to about 18% phosphatidylethanolamine. In embodiments, the lecithin comprises from 0% to about 2% phosphatidylethanolamine. In embodiments, the lecithin comprises from about 12% to about 18% phosphatidylethanolamine. In embodiments, the lecithin comprises about 14.6% phosphatidylethanolamine. In embodiments, the lecithin comprises a minimum of about 5% phosphatidylethanolamine.

In embodiments, the lecithin comprises lysophosphatidyl choline. In embodiments, the lecithin comprises from about 0% to about 10% lysophosphatidyl choline by weight. In embodiments, the lecithin comprises about 5%, about 6%, about 7%, about 8%, about 9%, about 10%, about 11%, about 12%, about 13%, about 14%, about 15%, about 16%, about 17%, about 18%, about 19%, about 20%, about 20%, about 21%, about 22%, about 23%, about 24%, about 25%, about 26%, about 27%, about 28%, about 29%, about 30% phosphatidylethanolamine by weight of the lecithin, including all ranges and values therebetween. In embodiments, the lecithin comprises a maximum of about 3% lysophosphatidyl choline by weight. In embodiments, the lecithin comprises a maximum of about 4% lysophosphatidyl choline by weight. In embodiments, the lecithin comprises a maximum of about 1.5% lysophosphatidyl choline by weight.

In embodiments, the lecithin comprises about 70% phosphatidylcholine, from 7 to about 10% phosphatidylethanolamine, and a maximum of 3% of lysophosphatidylcholine by weight of the lecithin. In embodiments, the lecithin comprises at least 45% phosphatidylcholine, from 10 to about 18% phosphatidylethanolamine, and a maximum of 4% of lysophosphatidylcholine by weight of the lecithin. In embodiments, the lecithin comprises about 92% phosphatidylcholine, from 0 to about 2% phosphatidylethanolamine, and a maximum of 3% of lysophosphatidylcholine by weight of the lecithin. In embodiments, the lecithin comprises about 70% phosphatidylcholine, from about 12% to about 18% phosphatidylethanolamine, and a maximum of 3% of lysophosphatidylcholine by weight of the lecithin. In embodiments, the lecithin comprises about 69.8% phosphatidylcholine, about 14.6% phosphatidylethanolamine, and about 1.5% of lysophosphatidylcholine by weight of the lecithin. In embodiments, the lecithin comprises a minimum of 50% of phosphatidylcholine and a minimum of 5% phosphatidylethanolamine by weight of the lecithin. In embodiments, the lecithin comprises a minimum of 20% of phosphatidylcholine and a minimum of 20% lysophosphatidyl choline by weight of the lecithin.

In embodiments, the aqueous ultrasound coupling agents described herein comprise from about 1 g/L to about 12 g/L lecithin. In embodiments, the aqueous ultrasound coupling agents described herein comprise from about 4.2 g/L to about 12 g/L lecithin. In embodiments, the aqueous ultrasound coupling agents described herein comprise from about 3.2 g/L to about 5.2 g/L lecithin. In embodiments, the aqueous ultrasound coupling agents described herein comprise up to about 12 g/L lecithin. In embodiments, the aqueous ultrasound coupling agents described herein comprise from about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L lecithin, or any concentration or subrange thereof. In embodiments, the ultrasound coupling agent comprises about 4.2 g/L lecithin.

In embodiments, the aqueous ultrasound coupling agents described herein comprise from about 0.1% w/v to about 1.2% w/v lecithin. In embodiments, the aqueous ultrasound coupling agents described herein comprise from about 0.4% w/v to about 1.2% w/v lecithin. In embodiments, the aqueous ultrasound coupling agents described herein comprise from about 0.3% w/v to about 0.6% w/v lecithin. In embodiments, the aqueous ultrasound coupling agents described herein comprise up to about 1.2% w/v lecithin. In embodiments, the ultrasound coupling agent comprises about 0.42% w/v lecithin.

In embodiments, the aqueous ultrasound coupling agents described herein comprise from about 1 g/L to about 12 g/L of phospholipid. In embodiments, the aqueous ultrasound coupling agents described herein comprise from about 4.2 g/L to about 12 g/L of phospholipid. In embodiments, the aqueous ultrasound coupling agents described herein comprise from about 3.2 g/L to about 5.2 g/L of phospholipid. In embodiments, the aqueous ultrasound coupling agents described herein comprise up to about 12 g/L of phospholipid. In embodiments, the aqueous ultrasound coupling agents described herein comprise from about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L of phospholipid, or any concentration or subrange thereof. In embodiments, the ultrasound coupling agent comprises about 4.2 g/L of phospholipid.

In embodiments, the aqueous ultrasound coupling agents described herein comprise from about 0.1% w/v to about 1.2% w/v of phospholipid. In embodiments, the aqueous ultrasound coupling agents described herein comprise from about 0.4% w/v to about 1.2% w/v of phospholipid. In embodiments, the aqueous ultrasound coupling agents described herein comprise from about 0.3% w/v to about 0.6% w/v of phospholipid. In embodiments, the aqueous ultrasound coupling agents described herein comprise up to about 1.2% w/v of phospholipid. In embodiments, the ultrasound coupling agent comprises about 0.42% w/v of phospholipid.

In embodiments, the aqueous ultrasound coupling agent comprises:
a) 60.0 to 80.0 g/L of at least one triglyceride; and
b) 3.8 to 4.5 g/L of at least one phospholipid.

In embodiments, the aqueous ultrasound coupling agent comprises:
a) 20.0 to 90.0 g/L soybean oil; and
b) 1.2 to 5.4 g/L lecithin.

In embodiments, the aqueous ultrasound coupling agent comprises:
a) 60.0 to 80.0 g/L soybean oil; and
b) 3.8 to 4.5 g/L lecithin.

In a preferred embodiment, the lecithin is egg yolk lecithin, soy lecithin or a mixture thereof.

### Humectants (e.g., Glycerol)

In embodiments, the aqueous ultrasound coupling agent comprises at least one humectant. As used herein, the term “humectant” refers to a hygroscopic substance which is capable of controlling the water content within a material. Humectants typically therefore comprise molecules having one or more hydrophilic groups, such as hydroxyl groups. In an embodiment, the humectant is a polyol.

In an embodiment, the aqueous ultrasound coupling agent comprises at least one humectant. In an embodiment, the humectant is biocompatible, sterile, and non-toxic. Any pharmaceutically acceptable humectant may be used. In a preferred embodiment, the at least one humectant is selected from the group consisting of polyols, lactic acid and mixtures thereof, especially polyols. Example of polyols include glycerol (also known as glycerin/glycerine), propylene glycol, and sorbitol. The use of glycerol is especially preferred.

In embodiments, the humectant is typically present in the aqueous ultrasound coupling agent in an amount of 2.2 to 9.5 g/L, 4.5 to 9.2 g/L, 5.5 to 9.0 g/L, or 6.5 to 8.5 g/L. Where a mixture of humectants is used, the amount of humectant refers to the sum of the amount of each humectant present in the aqueous ultrasound coupling agent. In embodiments, the humectant is biocompatible, sterile and non-toxic.

The humectant may contribute to controlling the osmotic pressure of the ultrasound coupling agent, as described herein.

In embodiments, the aqueous ultrasound coupling agent comprises:
a) 60.0 to 80.0 g/L of at least one triglyceride;
b) 3.8 to 4.5 g/L of at least one phospholipid; and
c) 6.5 to 8.5 g/L of at least one humectant.

In embodiments, the aqueous ultrasound coupling agent comprises:
a) 60.0 to 80.0 g/L of at least one triglyceride;
b) 3.8 to 4.5 g/L of at least one phospholipid; and
c) 6.5 to 8.5 g/L of glycerol.

In embodiments, the aqueous ultrasound coupling agent comprises:
a) 20.0 to 90.0 g/L soybean oil;
b) 1.2 to 5.4 g/L lecithin; and
c) 2.2 to 9.5 g/L glycerol.

In embodiments, the aqueous ultrasound coupling agent comprises:
a) 60.0 to 80.0 g/L soybean oil;
b) 3.8 to 4.5 g/L lecithin; and
c) 6.5 to 8.5 g/L glycerol.

In embodiments, the aqueous ultrasound coupling agent comprises:
a) 60.0 to 80.0 g/L soybean oil;
b) 3.8 to 4.5 g/L lecithin; and
c) 22 to 25 g/L glycerol.

In embodiments, the aqueous ultrasound coupling agents described herein comprise from about 8 g/L to about 26 g/L of glycerol. In embodiments, the aqueous ultrasound coupling agents described herein comprise from about 14 g/L to about 26 g/L of glycerol. In embodiments, the aqueous ultrasound coupling agents described herein comprise about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L glycerol, or any value or range therebetween. In embodiments, the aqueous ultrasound coupling agents described herein comprise about 16 g/L glycerol. In embodiments, the aqueous ultrasound coupling agents described herein comprise about 23.7 g/L glycerol.

### Additional Ingredients

In embodiments the aqueous ultrasound coupling agent comprises at least one pH-adjusting additive in order to adjust the pH of the agent to a level suitable for the intended clinical use. In one embodiment, the pH-adjusting additive (also called a “pH buffer”) is a metal hydroxide, preferably a group (I) metal hydroxide such as sodium hydroxide, potassium hydroxide or a mixture thereof. In a preferred embodiment, the pH-adjusting additive is sodium hydroxide. Alternatively, a base such as a tertiary amine (e.g. trimethylamine) can be added to the ultrasound coupling agent to adjust its pH. Optionally the aqueous ultrasound coupling agent (e.g. fluid) can be buffered, however the use of a buffer is not required.

In embodiments, the solvent used to prepare the aqueous ultrasound coupling agent is water.

In one embodiment, the coupling agent may be sterilized by gamma irradiation and adjusted to pH 9-12, preferably pH 10-11 prior to gamma irradiation. This is believed to compensate for the observed drop in pH when the formulation is irradiated and/or stored. In another embodiment, the aqueous ultrasound coupling agent may have a pH of 6.0 to 9.0, preferably 6.5 to 8.0 after sterilization by gamma irradiation and storage for at least 5 days. Most bacteria are neutrophiles and grow best at near neutral pH, i.e. around 7. Where the pH of the formulation after manufacturing but before irradiation is above 9.0 this will reduce as much as possible the risk of bacterial growth occurring between mixing and sterilization. The reduction of pH (e.g. to 6.5 to 8.0) after sterilization makes the formulation compatible for use without further adjustment.

In embodiments, where technically viable, the ultrasound coupling agent may be a fluid or a gel. Correspondingly, the coupling agent may additionally comprise any appropriate gelling agent. Examples of gelling agents are well known in the art and include protein-based gelling agents such as gelatine, and polysaccharide-based gelling agents such as pectin, agar and alginates. Mixtures of gelling agents may be used.

In embodiments, the ultrasound coupling agent is preferably biocompatible, sterile and non-toxic.

### C. Properties of Aqueous Ultrasound Coupling Agents

### Attenuation Coefficients

In embodiments, an aqueous ultrasound coupling agent described herein comprises an attenuation coefficient that is substantially similar (i.e., within 20%, 15%, 10% or 5%) of an attenuation coefficient of a tissue, organ, or cavity that is being imaged. In embodiments, an aqueous ultrasound coupling agent described herein comprises an attenuation coefficient that is the same as the attenuation coefficient of a tissue, organ, or cavity that is being imaged. In embodiments, using an aqueous ultrasound coupling agent that is substantially similar to or the same as an attenuation coefficient of a tissue, organ, or cavity that is being imaged results in a reduction of ultrasound artifacts.

In embodiments, an aqueous ultrasound coupling agent described herein comprises an attenuation coefficient that is within 5%, within 10%, within 15%, within 20%, within 25%, or within 30% of the attenuation coefficient of an attenuation coefficient of a tissue, organ, or cavity that is being imaged. For example, if the attenuation coefficient of a tissue, organ, or cavity is 1.5 dB/(MHz*cm), an aqueous ultrasound coupling agent that has an attenuation coefficient that is within 20% of the tissue, organ, or cavity has an attenuation coefficient from about 1.2-1.8 dB/(MHz*cm). In embodiments, an aqueous ultrasound coupling agent has an attenuation coefficient that is substantially similar to an attenuation coefficient of bone, brain, lung, liver, kidney, heart, cerebrospinal fluid, a soft tissue, a bladder, or a colon. The attenuation coefficient of various tissues and organs is shown in the table below:

| | Attenuation Coefficient |
|---|---|
| Organ or Tissue | (dB/(MHz*cm)) |
| Water | 0.002 |
| Bladder | 0.5-1.5 |
| Blood | 0.15 |
| Brain | 0.5-1.5 (e.g., 0.8) |
| Bone | 3-15 |
| Cerebrospinal fluid | 0.1-0.5 |
| Colon | 0.1-1.5 |
| Eye (lens) | 2 |
| Fat | 0.6 |
| Heart | 0.5-1.5 |
| Kidneys | 0.5-1.5 (e.g., 1) |
| Lung | 0.1-0.5 |
| Liver | 0.5-1.5 (e.g., 0.9) |
| Skull | 20 |
| Soft tissues (e.g., muscle, skin, fat) | 0.5-1.5 |

In embodiments, the attenuation coefficient of an aqueous ultrasound coupling agent described herein is from about 0.05 dB/(MHz*cm) to about 15 dB/(MHz*cm). In embodiments, the attenuation coefficient of an aqueous ultrasound coupling agent described herein is from about 3 dB/(MHz*cm) to about 15 dB/(MHz*cm). In embodiments, the attenuation coefficient of an aqueous ultrasound coupling agent described herein is from about 0.002 dB/(MHz*cm) to about 1.0 dB/(MHz*cm). In embodiments, the attenuation coefficient of an aqueous ultrasound coupling agent described herein is from about 0.05 dB/(MHz*cm) to about 1.8 dB/(MHz*cm). In embodiments, the attenuation coefficient of an aqueous ultrasound coupling agent described herein is from about 0.1 dB/(MHz*cm) to about 1.5 dB/(MHz*cm). In embodiments, the attenuation coefficient of an aqueous ultrasound coupling agent described herein is from about 0.5 dB/(MHz*cm) to about 1.5 dB/(MHz*cm). In embodiments, the attenuation coefficient of an aqueous ultrasound coupling agent described herein is from about 0.1 dB/(MHz*cm) to about 0.5 dB/(MHz*cm). In embodiments, the attenuation coefficient of an aqueous ultrasound coupling agent described herein is from about 0.1 dB/(MHz*cm) to about 0.7 dB/(MHz*cm). In embodiments, the attenuation coefficient of an aqueous ultrasound coupling agent described herein is from about 0.1 dB/(MHz*cm) to about 1.0 dB/(MHz*cm). In embodiments, the attenuation coefficient of an aqueous ultrasound coupling agent described herein is from about 0.3 dB/(MHz*cm) to about 0.7 dB/(MHz*cm). In embodiments, the attenuation coefficient of an aqueous ultrasound coupling agent described herein is from about 0.5 dB/(MHz*cm) to about 0.7 dB/(MHz*cm).

In embodiments, the attenuation coefficient of an aqueous ultrasound coupling agent described herein is about 0.001 dB/(MHz*cm), about 0.005 dB/(MHz*cm), about 0.01 dB/(MHz*cm), about 0.02 dB/(MHz*cm), about 0.03 dB/(MHz*cm), about 0.04 dB/(MHz*cm), about 0.05 dB/(MHz*cm), about 0.06 dB/(MHz*cm), about 0.07 dB/(MHz*cm), about 0.08 dB/(MHz*cm), about 0.09 dB/(MHz*cm), about 0.1 dB/(MHz*cm), about 0.15 dB/(MHz*cm), about 0.2 dB/(MHz*cm), about 0.25 dB/(MHz*cm), about 0.3 dB/(MHz*cm), about 0.35 dB/(MHz*cm), about 0.4 dB/(MHz*cm), about 0.45 dB/(MHz*cm), about 0.5 dB/(MHz*cm), about 0.55 dB/(MHz*cm), about 0.6 dB/(MHz*cm), about 0.65 dB/(MHz*cm), about 0.7 dB/(MHz*cm), about 0.75 dB/(MHz*cm), about 0.8 dB/(MHz*cm), about 0.85 dB/(MHz*cm), about 0.9 dB/(MHz*cm), about 0.95 dB/(MHz*cm), about 1 dB/(MHz*cm), about 1.05 dB/(MHz*cm), about 1.1 dB/(MHz*cm), about 1.15 dB/(MHz*cm), about 1.2 dB/(MHz*cm), about 1.25 dB/(MHz*cm), about 1.3 dB/(MHz*cm), about 1.35 dB/(MHz*cm), about 1.4 dB/(MHz*cm), about 1.45 dB/(MHz*cm), about 1.5 dB/(MHz*cm), about 1.55 dB/(MHz*cm), about 1.6 dB/(MHz*cm), about 1.65 dB/(MHz*cm), about 1.7 dB/(MHz*cm), about 1.75 dB/(MHz*cm), about 1.8 dB/(MHz*cm), about 1.85 dB/(MHz*cm), about 1.9 dB/(MHz*cm), about 1.95 dB/(MHz*cm), about 2 dB/(MHz*cm), about 2.05 dB/(MHz*cm), about 2.1 dB/(MHz*cm), about 2.15 dB/(MHz*cm), about 2.2 dB/(MHz*cm), about 2.25 dB/(MHz*cm), about 2.3 dB/(MHz*cm), about 2.35 dB/(MHz*cm), about 2.4 dB/(MHz*cm), about 2.45 dB/(MHz*cm), about 2.5 dB/(MHz*cm), about 2.55 dB/(MHz*cm), about 2.6 dB/(MHz*cm), about 2.65 dB/(MHz*cm), about 2.7 dB/(MHz*cm), about 2.75 dB/(MHz*cm), about 2.8 dB/(MHz*cm), about 2.85 dB/(MHz*cm), about 2.9 dB/(MHz*cm), about 2.95 dB/(MHz*cm), about 3 dB/(MHz*cm), about 3.05 dB/(MHz*cm), about 3.1 dB/(MHz*cm), about 3.15 dB/(MHz*cm), about 3.2 dB/(MHz*cm), about 3.25 dB/(MHz*cm), about 3.3 dB/(MHz*cm), about 3.35 dB/(MHz*cm), about 3.4 dB/(MHz*cm), about 3.45 dB/(MHz*cm), about 3.5 dB/(MHz*cm), about 3.55 dB/(MHz*cm), about 3.6 dB/(MHz*cm), about 3.65 dB/(MHz*cm), about 3.7 dB/(MHz*cm), about 3.75 dB/(MHz*cm), about 3.8 dB/(MHz*cm), about 3.85 dB/(MHz*cm), about 3.9 dB/(MHz*cm), about 3.95 dB/(MHz*cm), about 4 dB/(MHz*cm), about 4.05 dB/(MHz*cm), about 4.1 dB/(MHz*cm), about 4.15 dB/(MHz*cm), about 4.2 dB/(MHz*cm), about 4.25 dB/(MHz*cm), about 4.3 dB/(MHz*cm), about 4.35 dB/(MHz*cm), about 4.4 dB/(MHz*cm), about 4.45 dB/(MHz*cm), about 4.5 dB/(MHz*cm), about 4.55 dB/(MHz*cm), about 4.6 dB/(MHz*cm), about 4.65 dB/(MHz*cm), about 4.7 dB/(MHz*cm), about 4.75 dB/(MHz*cm), about 4.8 dB/(MHz*cm), about 4.85 dB/(MHz*cm), about 4.9 dB/(MHz*cm), about 4.95 dB/(MHz*cm), about 5 dB/(MHz*cm), about 5.05 dB/(MHz*cm), about 5.1 dB/(MHz*cm), about 5.15 dB/(MHz*cm), about 5.2 dB/(MHz*cm), about 5.25 dB/(MHz*cm), about 5.3 dB/(MHz*cm), about 5.35 dB/(MHz*cm), about 5.4 dB/(MHz*cm), about 5.45 dB/(MHz*cm), about 5.5 dB/(MHz*cm), about 5.55 dB/(MHz*cm), about 5.6 dB/(MHz*cm), about 5.65 dB/(MHz*cm), about 5.7 dB/(MHz*cm), about 5.75 dB/(MHz*cm), about 5.8 dB/(MHz*cm), about 5.85 dB/(MHz*cm), about 5.9 dB/(MHz*cm), about 5.95 dB/(MHz*cm), about 6 dB/(MHz*cm), about 6.05 dB/(MHz*cm), about 6.1 dB/(MHz*cm), about 6.15 dB/(MHz*cm), about 6.2 dB/(MHz*cm), about 6.25 dB/(MHz*cm), about 6.3 dB/(MHz*cm), about 6.35 dB/(MHz*cm), about 6.4 dB/(MHz*cm), about 6.45 dB/(MHz*cm), about 6.5 dB/(MHz*cm), about 6.55 dB/(MHz*cm), about 6.6 dB/(MHz*cm), about 6.65 dB/(MHz*cm), about 6.7 dB/(MHz*cm), about 6.75 dB/(MHz*cm), about 6.8 dB/(MHz*cm), about 6.85 dB/(MHz*cm), about 6.9 dB/(MHz*cm), about 6.95 dB/(MHz*cm), about 7 dB/(MHz*cm), about 7.05 dB/(MHz*cm), about 7.1 dB/(MHz*cm), about 7.15 dB/(MHz*cm), about 7.2 dB/(MHz*cm), about 7.25 dB/(MHz*cm), about 7.3 dB/(MHz*cm), about 7.35 dB/(MHz*cm), about 7.4 dB/(MHz*cm), about 7.45 dB/(MHz*cm), about 7.5 dB/(MHz*cm), about 7.55 dB/(MHz*cm), about 7.6 dB/(MHz*cm), about 7.65 dB/(MHz*cm), about 7.7 dB/(MHz*cm), about 7.75 dB/(MHz*cm), about 7.8 dB/(MHz*cm), about 7.85 dB/(MHz*cm), about 7.9 dB/(MHz*cm), about 7.95 dB/(MHz*cm), about 8 dB/(MHz*cm), about 8.05 dB/(MHz*cm), about 8.1 dB/(MHz*cm), about 8.15 dB/(MHz*cm), about 8.2 dB/(MHz*cm), about 8.25 dB/(MHz*cm), about 8.3 dB/(MHz*cm), about 8.35 dB/(MHz*cm), about 8.4 dB/(MHz*cm), about 8.45 dB/(MHz*cm), about 8.5 dB/(MHz*cm), about 8.55 dB/(MHz*cm), about 8.6 dB/(MHz*cm), about 8.65 dB/(MHz*cm), about 8.7 dB/(MHz*cm), about 8.75 dB/(MHz*cm), about 8.8 dB/(MHz*cm), about 8.85 dB/(MHz*cm), about 8.9 dB/(MHz*cm), about 8.95 dB/(MHz*cm), about 9 dB/(MHz*cm), about 9.05 dB/(MHz*cm), about 9.1 dB/(MHz*cm), about 9.15 dB/(MHz*cm), about 9.2 dB/(MHz*cm), about 9.25 dB/(MHz*cm), about 9.3 dB/(MHz*cm), about 9.35 dB/(MHz*cm), about 9.4 dB/(MHz*cm), about 9.45 dB/(MHz*cm), about 9.5 dB/(MHz*cm), about 9.55 dB/(MHz*cm), about 9.6 dB/(MHz*cm), about 9.65 dB/(MHz*cm), about 9.7 dB/(MHz*cm), about 9.75 dB/(MHz*cm), about 9.8 dB/(MHz*cm), about 9.85 dB/(MHz*cm), about 9.9 dB/(MHz*cm), about 9.95 dB/(MHz*cm), about 10 dB/(MHz*cm), about 10.05 dB/(MHz*cm), about 10.1 dB/(MHz*cm), about 10.15 dB/(MHz*cm), about 10.2 dB/(MHz*cm), about 10.25 dB/(MHz*cm), about 10.3 dB/(MHz*cm), about 10.35 dB/(MHz*cm), about 10.4 dB/(MHz*cm), about 10.45 dB/(MHz*cm), about 10.5 dB/(MHz*cm), about 10.55 dB/(MHz*cm), about 10.6 dB/(MHz*cm), about 10.65 dB/(MHz*cm), about 10.7 dB/(MHz*cm), about 10.75 dB/(MHz*cm), about 10.8 dB/(MHz*cm), about 10.85 dB/(MHz*cm), about 10.9 dB/(MHz*cm), about 10.95 dB/(MHz*cm), about 11 dB/(MHz*cm), about 11.05 dB/(MHz*cm), about 11.1 dB/(MHz*cm), about 11.15 dB/(MHz*cm), about 11.2 dB/(MHz*cm), about 11.25 dB/(MHz*cm), about 11.3 dB/(MHz*cm), about 11.35 dB/(MHz*cm), about 11.4 dB/(MHz*cm), about 11.45 dB/(MHz*cm), about 11.5 dB/(MHz*cm), about 11.55 dB/(MHz*cm), about 11.6 dB/(MHz*cm), about 11.65 dB/(MHz*cm), about 11.7 dB/(MHz*cm), about 11.75 dB/(MHz*cm), about 11.8 dB/(MHz*cm), about 11.85 dB/(MHz*cm), about 11.9 dB/(MHz*cm), about 11.95 dB/(MHz*cm), about 12 dB/(MHz*cm), about 12.05 dB/(MHz*cm), about t 12.1 dB/(MHz*cm), about 12.15 dB/(MHz*cm), about 12.2 dB/(MHz*cm), about 12.25 dB/(MHz*cm), about 12.3 dB/(MHz*cm), about 12.35 dB/(MHz*cm), about 12.4 dB/(MHz*cm), about 12.45 dB/(MHz*cm), about 12.5 dB/(MHz*cm), about 12.55 dB/(MHz*cm), about 12.6 dB/(MHz*cm), about 12.65 dB/(MHz*cm), about 12.7 dB/(MHz*cm), about 12.75 dB/(MHz*cm), about 12.8 dB/(MHz*cm), about 12.85 dB/(MHz*cm), about 12.9 dB/(MHz*cm), about 12.95 dB/(MHz*cm), about 13 dB/(MHz*cm), about 13.05 dB/(MHz*cm), about 13.1 dB/(MHz*cm), about 13.15 dB/(MHz*cm), about 13.2 dB/(MHz*cm), about 13.25 dB/(MHz*cm), about 13.3 dB/(MHz*cm), about 13.35 dB/(MHz*cm), about 13.4 dB/(MHz*cm), about 13.45 dB/(MHz*cm), about 13.5 dB/(MHz*cm), about 13.55 dB/(MHz*cm), about 13.6 dB/(MHz*cm), about 13.65 dB/(MHz*cm), about 13.7 dB/(MHz*cm), about 13.75 dB/(MHz*cm), about 13.8 dB/(MHz*cm), about 13.85 dB/(MHz*cm), about 13.9 dB/(MHz*cm), about 13.95 dB/(MHz*cm), about 14 dB/(MHz*cm), about 14.05 dB/(MHz*cm), about 14.1 dB/(MHz*cm), about 14.15 dB/(MHz*cm), about 14.2 dB/(MHz*cm), about 14.25 dB/(MHz*cm), about 14.3 dB/(MHz*cm), about 14.35 dB/(MHz*cm), about 14.4 dB/(MHz*cm), about 14.45 dB/(MHz*cm), about 14.5 dB/(MHz*cm), about 14.55 dB/(MHz*cm), about 14.6 dB/(MHz*cm), about 14.65 dB/(MHz*cm), about 14.7 dB/(MHz*cm), about 14.75 dB/(MHz*cm), about 14.8 dB/(MHz*cm), about 14.85 dB/(MHz*cm), about 14.9 dB/(MHz*cm), about 14.95 dB/(MHz*cm), about 15 dB/(MHz*cm), or any value or subrange therebetween. In embodiments, the attenuation coefficient of an aqueous ultrasound coupling agent described herein is about 0.5886

In embodiments, the purpose of the ultrasound coupling agent described herein is not only to avoid air pockets between the ultrasound transducer/probe and tissue to be imaged and to facilitate a good acoustic coupling between the probe and tissue, but also to minimize the appearance of artifacts by matching the attenuation of the ultrasound coupling agent and that of the tissue to be imaged. The attenuation of a particular tissue/ultrasound coupling agent is however in part a function of the frequency of the ultrasound used for imaging (as well as the propagation length), and so a more common parameter used to determine the degree to which the ultrasound coupling agent is suitable for use with certain tissue types is the attenuation coefficient (a). This has units dB/(MHz·cm) and is a property of the material itself i.e. is not dependent on frequency or propagation length. The attenuation coefficient is measured by measuring the amplitude of signals obtained using pulse-echo measurements applied to the ultrasound coupling agent and comparing this to the amplitude using pulse-echo measurements applied to a specific reference material (e.g. water).

In embodiments, the aqueous ultrasound coupling agent has an attenuation coefficient (α) that is at least a factor of 30 larger than the attenuation coefficient for water, (i.e. α>0.066 dB/(MHz*cm)). In one embodiment, the attenuation coefficient is from about 0.10 to about 1.10, about 0.15 to about 1.00 dB/(MHz·cm), or about 0.20 to about 0.90 dB/(MHz*cm). In embodiments, the attenuation coefficient is from about 0.30 to about 0.80 dB/(MHz*cm). In embodiments, the attenuation coefficient is from about 0.15 to about 0.60 dB/(MHz*cm). In embodiments, the coupling agent has an attenuation coefficient targeted at about 0.60 dB/(MHz cm) (±0.10 dB/MHz*cm), which is in the same order as the attenuation coefficient of the adult human brain.

In embodiments, the ultrasound coupling agent has an attenuation coefficient between that of water (i.e. 0.002 dB/(MHz*cm)) and the attenuation coefficient of the target tissue (i.e. the tissue to be imaged). For example, when the ultrasound coupling agent is for use in invasive ultrasound imaging of the brain, the attenuation constant of the ultrasound coupling agent is from about 0.002 to about 1 dB/(MHz*cm), from about 0.10 to about 0.60 dB/(MHz*cm), from about 0.002 to about 0.8 dB/(MHz*cm), or from about 0.15 to about 0.60 dB/(MHz*cm).

### Shelf Life

The “shelf life” of an aqueous ultrasound coupling agent described herein refers to the length of time in which the oil droplets within the coupling agent (e.g., the phospholipid and/or triglycerides) are homogenous with the water (i.e., when the oil droplets and water are not phase separated) of the aqueous ultrasound coupling agent. In embodiments, the shelf life of an aqueous ultrasound coupling agent is observed visually. The shelf life of an aqueous ultrasound coupling agent must be sufficient such that surgery or ultrasound imaging can be performed without the aqueous ultrasound coupling agent phase separating.

In embodiments, aqueous ultrasound coupling agents described herein have a shelf life of from about six months to about ten years, from about six months to about five years, from about 1 year to about 5 years, from about 2 years to about 5 years, from about 3 years to about 5 years, from about 6 months to about 4 years, from about 6 months to about 3 years, from about 6 months to about 2 years, from about six months to about one year, from about 1 year to about 4 years, from about 1 year to about 3 years, from about 1 year to about 2 years, or from about 2 years to about 4 years.

In embodiments, the shelf life is at least about 6 months, at least about 1 year, at least about 2 years, at least about 3 years, at least about 4 years, at least about 5 years, at least about 6 years, at least about 7 years, at least about 8 years, at least about 9 years, at least about 10 years, or more.

### Osmolality

The amount of solute (including any dissolved ions) in the ultrasound coupling agent can be expressed as the osmotic concentration (also known as osmolarity) of the ultrasound coupling agent. In embodiments, the ultrasound coupling agent has an osmotic concentration from about 200 to about 500 mOsm/kg, from about 225 to about 400 mOsm/kg, from about 250 to about 350 mOsm/kg, from about 275 to about 325 mOsm/kg, from about 270 to about 330 mOsm/kg, or from about 280 to about 300 mOsm/kg of aqueous ultrasound coupling agent. In In embodiments, the ultrasound coupling agent has an osmotic concentration of about 200 mOsm/kg, about 201 mOsm/kg, about 202 mOsm/kg, about 203 mOsm/kg, about 204 mOsm/kg, about 205 mOsm/kg, about 206 mOsm/kg, about 207 mOsm/kg, about 208 mOsm/kg, about 209 mOsm/kg, about 210 mOsm/kg, about 211 mOsm/kg, about 212 mOsm/kg, about 213 mOsm/kg, about 214 mOsm/kg, about 215 mOsm/kg, about 216 mOsm/kg, about 217 mOsm/kg, about 218 mOsm/kg, about 219 mOsm/kg, about 220 mOsm/kg, about 221 mOsm/kg, about 222 mOsm/kg, about 223 mOsm/kg, about 224 mOsm/kg, about 225 mOsm/kg, about 226 mOsm/kg, about 227 mOsm/kg, about 228 mOsm/kg, about 229 mOsm/kg, about 230 mOsm/kg, about 231 mOsm/kg, about 232 mOsm/kg, about 233 mOsm/kg, about 234 mOsm/kg, about 235 mOsm/kg, about 236 mOsm/kg, about 237 mOsm/kg, about 238 mOsm/kg, about 239 mOsm/kg, about 240 mOsm/kg, about 241 mOsm/kg, about 242 mOsm/kg, about 243 mOsm/kg, about 244 mOsm/kg, about 245 mOsm/kg, about 246 mOsm/kg, about 247 mOsm/kg, about 248 mOsm/kg, about 249 mOsm/kg, about 250 mOsm/kg, about 251 mOsm/kg, about 252 mOsm/kg, about 253 mOsm/kg, about 254 mOsm/kg, about 255 mOsm/kg, about 256 mOsm/kg, about 257 mOsm/kg, about 258 mOsm/kg, about 259 mOsm/kg, about 260 mOsm/kg, about 261 mOsm/kg, about 262 mOsm/kg, about 263 mOsm/kg, about 264 mOsm/kg, about 265 mOsm/kg, about 266 mOsm/kg, about 267 mOsm/kg, about 268 mOsm/kg, about 269 mOsm/kg, about 270 mOsm/kg, about 271 mOsm/kg, about 272 mOsm/kg, about 273 mOsm/kg, about 274 mOsm/kg, about 275 mOsm/kg, about 276 mOsm/kg, about 277 mOsm/kg, about 278 mOsm/kg, about 279 mOsm/kg, about 280 mOsm/kg, about 281 mOsm/kg, about 282 mOsm/kg, about 283 mOsm/kg, about 284 mOsm/kg, about 285 mOsm/kg, about 286 mOsm/kg, about 287 mOsm/kg, about 288 mOsm/kg, about 289 mOsm/kg, about 290 mOsm/kg, about 291 mOsm/kg, about 292 mOsm/kg, about 293 mOsm/kg, about 294 mOsm/kg, about 295 mOsm/kg, about 296 mOsm/kg, about 297 mOsm/kg, about 298 mOsm/kg, about 299 mOsm/kg, about 300 mOsm/kg, about 301 mOsm/kg, about 302 mOsm/kg, about 303 mOsm/kg, about 304 mOsm/kg, about 305 mOsm/kg, about 306 mOsm/kg, about 307 mOsm/kg, about 308 mOsm/kg, about 309 mOsm/kg, about 310 mOsm/kg, about 311 mOsm/kg, about 312 mOsm/kg, about 313 mOsm/kg, about 314 mOsm/kg, about 315 mOsm/kg, about 316 mOsm/kg, about 317 mOsm/kg, about 318 mOsm/kg, about 319 mOsm/kg, about 320 mOsm/kg, about 321 mOsm/kg, about 322 mOsm/kg, about 323 mOsm/kg, about 324 mOsm/kg, about 325 mOsm/kg, about 326 mOsm/kg, about 327 mOsm/kg, about 328 mOsm/kg, about 329 mOsm/kg, about 330 mOsm/kg, about 331 mOsm/kg, about 332 mOsm/kg, about 333 mOsm/kg, about 334 mOsm/kg, about 335 mOsm/kg, about 336 mOsm/kg, about 337 mOsm/kg, about 338 mOsm/kg, about 339 mOsm/kg, about 340 mOsm/kg, about 341 mOsm/kg, about 342 mOsm/kg, about 343 mOsm/kg, about 344 mOsm/kg, about 345 mOsm/kg, about 346 mOsm/kg, about 347 mOsm/kg, about 348 mOsm/kg, about 349 mOsm/kg, about 350 mOsm/kg, about 351 mOsm/kg, about 352 mOsm/kg, about 353 mOsm/kg, about 354 mOsm/kg, about 355 mOsm/kg, about 356 mOsm/kg, about 357 mOsm/kg, about 358 mOsm/kg, about 359 mOsm/kg, about 360 mOsm/kg, about 361 mOsm/kg, about 362 mOsm/kg, about 363 mOsm/kg, about 364 mOsm/kg, about 365 mOsm/kg, about 366 mOsm/kg, about 367 mOsm/kg, about 368 mOsm/kg, about 369 mOsm/kg, about 370 mOsm/kg, about 371 mOsm/kg, about 372 mOsm/kg, about 373 mOsm/kg, about 374 mOsm/kg, about 375 mOsm/kg, about 376 mOsm/kg, about 377 mOsm/kg, about 378 mOsm/kg, about 379 mOsm/kg, about 380 mOsm/kg, about 381 mOsm/kg, about 382 mOsm/kg, about 383 mOsm/kg, about 384 mOsm/kg, about 385 mOsm/kg, about 386 mOsm/kg, about 387 mOsm/kg, about 388 mOsm/kg, about 389 mOsm/kg, about 390 mOsm/kg, about 391 mOsm/kg, about 392 mOsm/kg, about 393 mOsm/kg, about 394 mOsm/kg, about 395 mOsm/kg, about 396 mOsm/kg, about 397 mOsm/kg, about 398 mOsm/kg, about 399 mOsm/kg, about 400 mOsm/kg, about 401 mOsm/kg, about 402 mOsm/kg, about 403 mOsm/kg, about 404 mOsm/kg, about 405 mOsm/kg, about 406 mOsm/kg, about 407 mOsm/kg, about 408 mOsm/kg, about 409 mOsm/kg, about 410 mOsm/kg, about 411 mOsm/kg, about 412 mOsm/kg, about 413 mOsm/kg, about 414 mOsm/kg, about 415 mOsm/kg, about 416 mOsm/kg, about 417 mOsm/kg, about 418 mOsm/kg, about 419 mOsm/kg, about 420 mOsm/kg, about 421 mOsm/kg, about 422 mOsm/kg, about 423 mOsm/kg, about 424 mOsm/kg, about 425 mOsm/kg, about 426 mOsm/kg, about 427 mOsm/kg, about 428 mOsm/kg, about 429 mOsm/kg, about 430 mOsm/kg, about 431 mOsm/kg, about 432 mOsm/kg, about 433 mOsm/kg, about 434 mOsm/kg, about 435 mOsm/kg, about 436 mOsm/kg, about 437 mOsm/kg, about 438 mOsm/kg, about 439 mOsm/kg, about 440 mOsm/kg, about 441 mOsm/kg, about 442 mOsm/kg, about 443 mOsm/kg, about 444 mOsm/kg, about 445 mOsm/kg, about 446 mOsm/kg, about 447 mOsm/kg, about 448 mOsm/kg, about 449 mOsm/kg, about 450 mOsm/kg, about 451 mOsm/kg, about 452 mOsm/kg, about 453 mOsm/kg, about 454 mOsm/kg, about 455 mOsm/kg, about 456 mOsm/kg, about 457 mOsm/kg, about 458 mOsm/kg, about 459 mOsm/kg, about 460 mOsm/kg, about 461 mOsm/kg, about 462 mOsm/kg, about 463 mOsm/kg, about 464 mOsm/kg, about 465 mOsm/kg, about 466 mOsm/kg, about 467 mOsm/kg, about 468 mOsm/kg, about 469 mOsm/kg, about 470 mOsm/kg, about 471 mOsm/kg, about 472 mOsm/kg, about 473 mOsm/kg, about 474 mOsm/kg, about 475 mOsm/kg, about 476 mOsm/kg, about 477 mOsm/kg, about 478 mOsm/kg, about 479 mOsm/kg, about 480 mOsm/kg, about 481 mOsm/kg, about 482 mOsm/kg, about 483 mOsm/kg, about 484 mOsm/kg, about 485 mOsm/kg, about 486 mOsm/kg, about 487 mOsm/kg, about 488 mOsm/kg, about 489 mOsm/kg, about 490 mOsm/kg, about 491 mOsm/kg, about 492 mOsm/kg, about 493 mOsm/kg, about 494 mOsm/kg, about 495 mOsm/kg, about 496 mOsm/kg, about 497 mOsm/kg, about 498 mOsm/kg, about 499 mOsm/kg, or about 500 mOsm/kg, including all values and ranges therebetween.

In embodiments, the aqueous ultrasound coupling agents have an osmolality of from about 270 mOsm/kg to about 330 mOsm/kg. In embodiments, the aqueous ultrasound coupling agents have an osmolality of from about 290 mOsm/kg to about 311 mOsm/kg. In embodiments, the aqueous ultrasound coupling agents have an osmolality of from about 290 mOsm/kg to about 305 mOsm/kg. In embodiments, the aqueous ultrasound coupling agents have an osmolality of about 270 mOsm/kg, about 275 mOsm/kg, about 280 mOsm/kg, about 285 mOsm/kg, about 290 mOsm/kg, about 295 mOsm/kg, about 300 mOsm/kg, about 305 mOsm/kg, about 310 mOsm/kg, about 320 mOsm/kg, about 325 mOsm/kg, or about 330 mOsm/kg, including all values and ranges therebetween. In embodiments, the aqueous ultrasound coupling agents have an osmolality of about Unless otherwise indicated, the osmolality of the aqueous ultrasound coupling agents described herein is measured on an OSMOMAT 030-D-D3P from GonoTec®, Ser. No. 08 02 22 osmometer.

In embodiments, the aqueous ultrasound coupling agent is isotonic with the fluid, tissue, or organ that is being imaged. In embodiments, the aqueous ultrasound coupling agent is isotonic with cerebral spinal fluid (~290-292 mOsm/kg).

pH

In embodiments, the aqueous ultrasound coupling agent should have a pH which matches the pH of the tissue to which it is intended to be contacted with or is substantially similar to the pH of the tissue that it contacts. In embodiments, the aqueous ultrasound coupling agent has a pH is from about 6.0 to about 9.0, from about 6.5 to about 8.5, or from about 6.5 to about 8.0. In embodiments, the pH is about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, or any pH or range therebetween. In embodiments, the aqueous ultrasound coupling agents described herein have a pH from about 6.5 to about 8.5, from about 6.5 to about 8, from about 6.9 to about 7.5, or from about 6.9 to about 7.5. In embodiments, the pH of the aqueous ultrasound coupling agent is about 6.9. In embodiments, the pH of the aqueous ultrasound coupling agent is about 7.3. In embodiments, the pH of the aqueous ultrasound coupling agent is about 7.5. In embodiments, the pH of the aqueous ultrasound coupling agent is substantially similar to the pH of cerebrospinal fluid (~pH 7.3).

The skilled person knows that the pH of the composition may gradually change over time depending on storage conditions etc. For example, in general the pH of the composition directly after manufacture is higher than that of the composition after a period of prolonged storage. For the avoidance of doubt therefore, by the pH of the composition is meant the pH of the composition shortly before its intended application (i.e., about 1 hour), as it is this that is important in determining the suitability of the composition for use on certain tissue types.

### Droplet Size

In embodiments, the triglycerides and phospholipids typically form an emulsion when added to a suitable solvent. In embodiments, triglycerides and phospholipids of the ultrasound coupling agent form an oil-in-water (o/w) type emulsion i.e, wherein droplets of oil (comprising at least component a)) are dispersed within a continuous aqueous phase. The amphiphilic phospholipid component b) helps to stabilize the interface formed between the dispersed triglyceride/oil phase and the continuous aqueous phase.

In embodiments, the droplet size of the emulsion can be tuned by varying the nature and/or relative amount of triglyceride and phospholipid components (as well as any optional co-emulsifier(s) that may be present). Additionally or alternatively, the droplet size can be tuned by controlling the shear forces applied during emulsification.

In embodiments, the number average (mean) droplet diameter is from about 100 nm to about 1000 nm, from about 200 nm to about 700 nm, or from about 230 nm to about 340 nm, including all values and ranges therebetween. In embodiments, the number average droplet diameter is from about 250 nm to about 320 nm, such as about 285 nm±35 nm, preferably 285 nm±5 nm. In embodiments, the number average droplet diameter is 285 nm±20%. Average droplet diameters are measured by Dynamic Light Scattering using a Malvern Zetasizer.

In embodiments, the aqueous ultrasound coupling agent has a volume mean weighted droplet size (i.e., diameter) of from about 0.2 μm to 5 μm, from about 0.2 μm to 4.5 μm, from about 0.2 μm to about 4 μm, from about 0.2 μm to about 3.5 μm, from about 0.2 μm to about 3 μm, from about 0.2 μm to about 2.5 μm, from about 0.2 μm to about 2 μm, from about 0.2 μm to about 1.5 μm, from about 0.2 μm to about 1 μm, from about 0.2 μm to about 0.9 μm, from about 0.2 μm to about 0.8 μm, from about 0.2 μm to about 0.7 μm, or from about 0.2 μm to about 0.6 μm. In embodiments, the aqueous ultrasound coupling agent has a volume mean weighted droplet size of from about 0.2 μm to about 0.55 μm. In embodiments, the aqueous ultrasound coupling agent has a volume mean weighted droplet size of from about 0.26 μm to about 0.50 μm. In embodiments, the aqueous ultrasound coupling agent has a volume mean weighted droplet size of from about 228 nm to about 342 nm. In embodiments, the aqueous ultrasound coupling agent has a volume mean weighted droplet size of about 258 nm. In embodiments, volume mean weighted droplet size is measured by laser diffraction.

In embodiments, the aqueous ultrasound coupling agent forms oil droplets, wherein 100% of the oil droplets have a diameter of less than 5 μm, less than 4.5 μm, less than 4 μm, less than 3.5 μm, less than 3 μm, less than 2.5 μm, less than 2 μm, less than 1.5 μm, less than 1 μm, less than 0.9 μm, less than 0.8 μm, less than 0.7 μm, less than 0.6 μm, or less than 0.5 μm. In embodiments, 100% of the oil droplets have a diameter that is less than 0.84 μm. In embodiments, 100% of the oil droplets have a diameter that is less than 0.96 μm. In embodiments, 100% of the oil droplets have a diameter that is less than 1.91 μm. In embodiments, none (of the droplets comprise a diameter of greater than 5000 nm.

In embodiments, the aqueous ultrasound coupling agent forms oil droplets with an average size of from about 200 nm to about 750 nm or from about 250 nm to about 500 nm. In embodiments, the aqueous ultrasound coupling agent forms oil droplets with an average size of about 285 nm±20% . . . . In embodiments, the aqueous ultrasound coupling agent forms oil droplets with an average size of about 369 nm. In embodiments, size is measured by dynamic light scattering. In embodiments, the size is measured by laser diffraction.

In embodiments, the droplets in the emulsion are monodisperse or essentially monodisperse. As used herein, the term “monodisperse” means that at least 90% by number of the droplets (D90) have a particular diameter or less. In embodiments, the D90 is in the range of 200 to 400 nm. In embodiments the D90 of the emulsion is in the range of 220 to 350 nm. In embodiments, the D90 of the emulsion is in the range of 230 to 340 nm. In embodiments, the D90 is from about 0.2 μm to 5 μm, from about 0.2 μm to 4.5 μm, from about 0.2 μm to about 4 μm, from about 0.2 μm to about 3.5 μm, from about 0.2 μm to about 3 μm, from about 0.2 μm to about 2.5 μm, from about 0.2 μm to about 2 μm, from about 0.2 μm to about 1.5 μm, from about 0.2 μm to about 1 μm, from about 0.2 μm to about 0.9 μm, from about 0.2 μm to about 0.8 μm, from about 0.2 μm to about 0.7 μm, about 0.2 μm to about 0.6 μm, from about 0.2 μm to about 1 μm, or from about 0.4 μm to about 1.4 μm. In embodiments, the D90 is about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 369 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, about 700 nm, about 710 nm, about 720 nm, about 730 nm, about 740 nm, about 750 nm, about 760 nm, about 770 nm, about 780 nm, about 790 nm, about 800 nm, about 810 nm, about 820 nm, about 830 nm, about 840 nm, about 850 nm, about 860 nm, about 870 nm, about 880 nm, about 890 nm, about 900 nm, about 910 nm, about 920 nm, about 930 nm, about 940 nm, about 950 nm, about 960 nm, about 970 nm, about 980 nm, about 990 nm, about 1000 nm, about 1010 nm, about 1020 nm, about 1030 nm, about 1040 nm, about 1050 nm, about 1060 nm, about 1070 nm, about 1080 nm, about 1090 nm, about 1100 nm, about 1110 nm, about 1120 nm, about 1130 nm, about 1140 nm, about 1150 nm, about 1160 nm, about 1170 nm, about 1180 nm, about 1190 nm, about 1200 nm, about 1210 nm, about 1220 nm, about 1230 nm, about 1240 nm, about 1250 nm, about 1260 nm, about 1270 nm, about 1280 nm, about 1290 nm, about 1300 nm, about 1310 nm, about 1320 nm, about 1330 nm, about 1340 nm, about 1350 nm, about 1360 nm, about 1370 nm, about 1380 nm, about 1390 nm, about 1400 nm, about 1410 nm, about 1420 nm, about 1430 nm, about 1440 nm, about 1450 nm, about 1460 nm, about 1470 nm, about 1480 nm, about 1490 nm, about 1500 nm, about 1510 nm, about 1520 nm, about 1530 nm, about 1540 nm, about 1550 nm, about 1560 nm, about 1570 nm, about 1580 nm, about 1590 nm, about 1600 nm, about 1610 nm, about 1620 nm, about 1630 nm, about 1640 nm, about 1650 nm, about 1660 nm, about 1670 nm, about 1680 nm, about 1690 nm, about 1700 nm, about 1710 nm, about 1720 nm, about 1730 nm, about 1740 nm, about 1750 nm, about 1760 nm, about 1770 nm, about 1780 nm, about 1790 nm, about 1800 nm, about 1810 nm, about 1820 nm, about 1830 nm, about 1840 nm, about 1850 nm, about 1860 nm, about 1870 nm, about 1880 nm, about 1890 nm, about 1900 nm, about 1910 nm, about 1920 nm, about 1930 nm, about 1940 nm, about 1950 nm, about 1960 nm, about 1970 nm, about 1980 nm, about 1990 nm, about 2000 nm, about 2010 nm, about 2020 nm, about 2030 nm, about 2040 nm, about 2050 nm, about 2060 nm, about 2070 nm, about 2080 nm, about 2090 nm, about 2100 nm, about 2110 nm, about 2120 nm, about 2130 nm, about 2140 nm, about 2150 nm, about 2160 nm, about 2170 nm, about 2180 nm, about 2190 nm, about 2200 nm, about 2210 nm, about 2220 nm, about 2230 nm, about 2240 nm, about 2250 nm, about 2260 nm, about 2270 nm, about 2280 nm, about 2290 nm, about 2300 nm, about 2310 nm, about 2320 nm, about 2330 nm, about 2340 nm, about 2350 nm, about 2360 nm, about 2370 nm, about 2380 nm, about 2390 nm, about 2400 nm, about 2410 nm, about 2420 nm, about 2430 nm, about 2440 nm, about 2450 nm, about 2460 nm, about 2470 nm, about 2480 nm, about 2490 nm, about 2500 nm, about 2510 nm, about 2520 nm, about 2530 nm, about 2540 nm, about 2550 nm, about 2560 nm, about 2570 nm, about 2580 nm, about 2590 nm, about 2600 nm, about 2610 nm, about 2620 nm, about 2630 nm, about 2640 nm, about 2650 nm, about 2660 nm, about 2670 nm, about 2680 nm, about 2690 nm, about 2700 nm, about 2710 nm, about 2720 nm, about 2730 nm, about 2740 nm, about 2750 nm, about 2760 nm, about 2770 nm, about 2780 nm, about 2790 nm, about 2800 nm, about 2810 nm, about 2820 nm, about 2830 nm, about 2840 nm, about 2850 nm, about 2860 nm, about 2870 nm, about 2880 nm, about 2890 nm, about 2900 nm, about 2910 nm, about 2920 nm, about 2930 nm, about 2940 nm, about 2950 nm, about 2960 nm, about 2970 nm, about 2980 nm, about 2990 nm, or about 3000 nm, including all values and ranges therebetween.

### D. Containers and Kits Containing Aqueous Ultrasound Coupling Agents

In embodiments, provided herein is a pre-filled syringe containing an ultrasound coupling agent described herein. A suitable syringe may be, for example, 10 to 250 ml in capacity, such as 25 to 100 ml in capacity. In one embodiment, the ultrasound coupling agent is sterilized by gamma irradiation of the pre-filled syringe containing said coupling agent. Pre-filled syringes may optionally be provided with a filter to reduce the probability that larger droplets and any presence of larger particles in the ultrasound coupling agent can enter the cavity during use. Typical filters will have a pore size around 0.1 to 5 μm, preferably 0.8 to 1.5 μm.

Advantageously, the kit is suitable for carrying out the method for preparing or method for formulating the aqueous ultrasound coupling agent as defined herein.

In another aspect, provided herein is a kit comprising: a mixture comprising at least one triglyceride, at least one phospholipid and optionally at least one humectant; and water. The mixture and aqueous solution are preferably as defined in any aspect described herein.

In embodiments, provided herein are kits comprising an aqueous ultrasound coupling agent and a container. In embodiments, the container is a syringe. In embodiments, the container is glass. In embodiments, the container is a bag. In embodiments, the container lacks one or more of dyes, preservatives, rubber, butyl, silica, polyvinyl chloride (PVC), and di (2-ethylhexyl) phthalate (DEHP). In embodiments, the container contains polypropylene, polyethylene, polycarbonate, cyclic olefin copolymer, cyclic olefin polymer, polymethyl methacrylate, or glass. In embodiments, the container contains cyclin olefin copolymer and cyclic olefin polymer.

In embodiments, provided herein are kits comprising an aqueous ultrasound coupling agent, a container, and a filter. In embodiments, the pore size of the filter is small enough to prevent microbial growth and large enough to allow oil droplets from the aqueous ultrasound coupling agent to pass through the filter. In embodiments, the filter comprises pores that are greater than 0.5 μm and smaller than 5 μm. In embodiments, the filter has a pore size of about 0.6 μm, about 0.7 μm, about 0.8 μm, about 0.9 μm, about 1 μm, about 1.1 μm, about 1.2 μm, about 1.3 μm, about 1.4 μm, about 1.5 μm, about 1.6 μm, about 1.7 μm, about 1.8 μm. about 1.9 μm, about 2 μm, about 2.1 μm, about 2.2 μm, about 2.3 μm, about 2.4 μm, about 2.5 μm, about 2.6 μm, about 2.7 μm, about 2.8 μm, about 2.9 μm, about 3 μm, about 3.1 μm, about 3.2 μm, about 3.3 μm, about 3.4 μm, about 3.5 μm, about 3.6 μm, about 3.7 μm, about 3.8 μm, about 3.9 μm, about 4 μm, about 4.1 μm, about 34.2 μm, about 4.3 μm, about 4.4 μm, about 4.5 μm, about 4.6 μm, about 4.7 μm, about 4.8 μm, about 4.9 μm, or about 5 μm, including all values and ranges therebetween. In embodiments, the filter has a pore size of about 2 μm. In embodiments, the filter has a pore size of about 1.2 μm.

In embodiments, the kit comprise a bag spike and a bag containing the aqueous ultrasound coupling agent. A bag spike is used to puncture the bag containing the aqueous ultrasound coupling agent. In embodiments the kit further comprises an inline, vented filter. Inline filters are placed directly in the bag. In embodiments, the filter is vented to allow air to escape from the fluid path and to prevent air bubbles from entering the bloodstreams and causing embolisms.

In embodiments, the kit comprise an aqueous ultrasound coupling agent, a syringe, a bag spike, and a filter. In embodiments, the filter is a 1.2 μm filter.

In embodiments, provided herein are containers comprising an aqueous ultrasound coupling agent described herein. In embodiments, the container is a syringe. In embodiments, the container is glass. In embodiments, the container is a bag. In embodiments, the container lacks one or more of dyes, preservatives, rubber, butyl, silica, polyvinyl chloride (PVC), and di (2-ethylhexyl) phthalate (DEHP). In embodiments, the container contains polypropylene, polyethylene, polycarbonate, cyclic olefin copolymer, cyclic olefin polymer, polymethyl methacrylate, or glass. In embodiments, the container contains cyclin olefin copolymer and cyclic olefin polymer.

In embodiments, the container is a syringe. In embodiments, the syringe is a 50 ml syringe. In embodiments, the syringe contains one or more of polypropylene, polyethylene, polycarbonate, cyclic olefin copolymer, cyclic olefin polymer, polymethyl methacrylate, or glass. In embodiments, the syringe comprises a plunger. In embodiments, the plunger comprises one or more of rubber (e.g., natural or synthetic), thermoplastic elastomers, silicone rubber, polyisoprene, ethylene propylene diene monomer, chlorobutyl rubber, or bromobutyl rubber.

In embodiments, the container is a bag. In embodiments, an aqueous ultrasound coupling agent is present in a sterile inline bag spike and infusion kit. In embodiments, the bag is a pressure bag.

### E. Methods of Using Aqueous Ultrasound Coupling Agents

In embodiments, the aqueous ultrasound coupling agent is for use in invasive ultrasound imaging of a subject. Examples of such uses include invasive ultrasound imaging during surgery, as well as non-surgical uses such as invasive diagnostic procedures (e.g. colorectal examination).

Various tissues/materials may be imaged using the ultrasound coupling agents described herein. In embodiments, the ultrasound coupling agent is for use in invasive ultrasound imaging of the brain, such as during brain surgery. In embodiments, the brain contains a tumor. In embodiments, an aqueous ultrasound coupling agent described herein is used to image a tumor via ultrasound. In embodiments, the tumor is resected. In embodiments, use of an aqueous ultrasound coupling agent allows a neurosurgeon to ensure removal of tumor tissue and not healthy tissue.

In embodiments, the ultrasound coupling agent is for use in invasive ultrasound imaging of other tissues such as heart tissue or breast tissue. In embodiments, the aqueous coupling agent described herein has an attenuation coefficient which makes it particularly suitable for imaging of the heart i.e. which ultrasound coupling agent (e.g. fluid) generates fewer artifacts during invasive ultrasound imaging of the heart.

In embodiments, provided herein is a method of ultrasound imaging of a subject, comprising administering to the subject an ultrasound coupling agent described herein through an opening in the body of the subject, applying ultrasound to said ultrasound coupling agent, and detecting ultrasound reflected from said ultrasound coupling agent. In embodiments, the method is a method of ultrasound imaging of a subject during surgery, such as brain surgery, breast cancer surgery, or open-heart surgery. In embodiments, the method is a method of ultrasound imaging of a subject during an invasive diagnostic procedure, such as colorectal examination.

In embodiments, provided herein is an aqueous ultrasound coupling agent comprising:
a) 20.0 to 90.0 g/L of at least one triglyceride;
b) 1.2 to 5.4 g/L of at least one phospholipid; and
c) optionally at least one humectant; for use in invasive ultrasound imaging of the heart, such as during open-heart surgery, and wherein the attenuation coefficient (a) of the ultrasound coupling agent is in the range of 0.1 to 1.0 dB/(MHz·cm), preferably 0.1 to 0.8 dB(MHz·cm). Unexpectedly, when the aqueous ultrasound coupling agent having an attenuation coefficient within this range is used in invasive ultrasound imaging of the heart, the intensity and prevalence of image artifacts such as brightness artifacts is reduced.

In embodiments, provided herein is an aqueous ultrasound coupling agent comprising:
a) 20.0 to 90.0 g/L of at least one triglyceride;
b) 1.2 to 5.4 g/L of at least one phospholipid; and
c) optionally at least one humectant;

for use in invasive ultrasound imaging of the breast, such as during breast cancer surgery, wherein the attenuation coefficient (a) of the agent is in the range of 0.1 to 1.2 dB/(MHz·cm), preferably 0.1 to 0.8 dB(MHz·cm). Unexpectedly, when the aqueous ultrasound coupling agent having an attenuation coefficient within this range is used in invasive ultrasound imaging of the breast, the intensity and prevalence of image artefacts such as brightness artifacts is reduced. The nature and amount of each of components a)-d) in the aqueous ultrasound coupling agent of this aspect may be as recited for the aqueous ultrasound coupling agent of any other aspect described herein.

In embodiments, provided herein is an aqueous ultrasound coupling agent for ultrasound imaging of the brain, comprising:
a) about 60 g/L to about 80.0 g/L of at least one triglyceride;
b) about 1.2 g/L to 12 g/L of at least one phospholipid; and
c) about 21 g/L to about 26 g/L of glycerol.

In embodiments, provided herein is an aqueous ultrasound coupling agent as hereinbefore defined for use in invasive ultrasound imaging of the colon in colorectal cancer staging using a balloon stand-off system, wherein the attenuation coefficient (a) of the ultrasound coupling agent is in the range of 0.1 to 0.6 dB(MHz·cm). Unexpectedly, when the aqueous ultrasound coupling agent having an attenuation coefficient within this range is used in colorectal cancer staging using a balloon stand-off system, the intensity and prevalence of image artefacts such as brightness artifacts is reduced.

The balloon standoff may comprise an outer “balloon” sheath surrounding at least a part of an ultrasound transducer and a liquid or gel ultrasound coupling material between the ultrasound transducer and the sheath. In one embodiment, the volume of ultrasound coupling material between the ultrasound transducer and the sheath may be varied in order to allow ease of positioning and conform the shape of the balloon to the biological structure being examined. The ultrasound coupling material of this embodiment will be the aqueous ultrasound coupling agent as described herein in any appropriate aspect or embodiment. The material of the sheath may be any appropriate material, such as an elastomeric polymer material. In one embodiment the material of the sheath may be latex-free.

In one aspect, the ultrasound coupling agent is for use in the invasive ultrasound imaging of the brain, heart, breast, veins, or colon.

In embodiments, provided herein are methods comprising: (i) contacting an organ or a cavity with an aqueous ultrasound coupling agent described herein; and (ii) obtaining an ultrasound image of the organ or the cavity. In embodiments, the organ or cavity comprises a tumor. In embodiments, the method further comprises resecting the tumor. In embodiments, the cavity is selected from any one of oral cavity, nasal cavity, orbital cavity, thoracic cavity, pericardial cavity, pleural cavities, abdominal cavity, pelvic cavity, cranial cavity, spinal cavity, articular cavities, synovial cavities, buccal cavity, cranial fossae, mandibular fossa, maxillary sinus, frontal sinus, ethmoidal sinus, sphenoidal sinus, mastoid air cells, auditory canal, middle ear cavity, tympanic cavity, vestibular system, cochlear duct, olfactory epithelium, oral vestibule, palatine tonsil, pharyngeal tonsil, lingual tonsil, sinus venosus, infundibulum, alveolar sacs, bronchioles, laryngeal cavity, tracheal cavity, gastric pits, crypts of lieberkühn, gallbladder fossa, renal sinus, ureteral opening, bladder trigone, ovarian follicles, testicular lobules, vaginal fornix, anal canal, vertebral foramen, intervertebral foramina, foramen magnum, and jugular foramen. In embodiments, the organ is selected from any one of brain, heart, lungs, liver, kidneys, stomach, small intestine, large intestine, pancreas, spleen, gallbladder, colon, spine, bladder, skin, eyes, ears, nose, mouth, tongue, throat, trachea, breast, esophagus, diaphragm, adrenal gland, thyroid, parathyroid gland, pituitary gland, pineal gland, hypothalamus, ovaries, testes, uterus, prostate, penis, vagina, cervix, urethra, rectum, anus, skeletal muscles, smooth muscle, cardiac muscle, bone marrow, bones, blood vessel, lymph node, lymphatic vessel, tonsils, appendix, adipose tissue, nerves, and connective tissue. In embodiments, the organ is the brain.

### F. Methods of Preparing Aqueous Ultrasound Coupling Agents

In one aspect, provided herein is a method of preparing an aqueous ultrasound coupling agent: the method comprising:
i) providing a mixture comprising at least one triglyceride, at least one phospholipid and optionally at least one humectant; and
ii) diluting said mixture with water.

The mixture provided in step i) is preferably an aqueous solution of the at least one triglyceride, at least one phospholipid and optionally at least one humectant. Optionally further components such as a pH-adjusting additive can also be present in the mixture. As to the nature of the triglyceride, phospholipid and optional humectant components, these can be any of the options recited in earlier aspects. A mixture comprising soybean oil, lecithin, and optionally glycerol is particularly preferred.

The amount of water=used to dilute said mixture can be adjusted so as to achieve the desired concentrations of triglyceride, phospholipid, and optional other components in the ultrasound coupling agent.

In one embodiment, any of the methods described herein include sterilization of the ultrasound coupling agent (of any embodiment disclosed herein). This may be by heat, gamma irradiation etc. In embodiments, sterilization comprises gamma irradiation and the radiation dose is from 10 to 100 kGy. In embodiments, method comprises filling the ultrasound coupling agent into a syringe and sterilizing the ultrasound coupling agent by gamma irradiation of a pre-filled syringe containing said coupling agent.

In embodiments, provided herein is a method of formulating an aqueous ultrasound coupling agent described herein, the method comprising:
i) providing a mixture comprising at least one triglyceride, at least one phospholipid and optionally at least one humectant;
ii) determining the attenuation coefficient (a) of the mixture;
iii) calculating the difference (d) between the attenuation coefficient of the mixture determined in step ii) and a reference attenuation coefficient (ar);
iv) forming a diluted mixture by diluting the mixture provided in step i) with water, in an amount which is determined by a function f(d) of the difference (d) calculated in step iii); and
v) optionally repeating steps ii)-iv) on the diluted mixture obtained in step iv).

By starting with a concentrated mixture of at least one triglyceride, at least one phospholipid and optionally other components, it is possible to arrive at an aqueous ultrasound coupling agent (generally a fluid) having the desired attenuation coefficient i.e. the reference attenuation coefficient, simply by varying the amount of water or aqueous solution added to the mixture. Preferably the reference attenuation coefficient (ar) corresponds to the attenuation coefficient of a target tissue in a subject to be imaged, such as brain tissue, heart tissue or breast tissue. The value of these reference attenuation coefficients for any particular tissue may be found in published reference tables. The nature of the mixture/aqueous solution may be the same as in the method for preparing the aqueous ultrasound coupling agent according to the aspect previously described. Preferably the mixture comprises soybean oil, lecithin and glycerol.

Regarding the function f(d) used to determine the amount of water/aqueous solution to be added to the mixture to form the diluted mixture, this may be determined experimentally, e.g. using calibration curves etc. In one embodiment, the function is a linear function i.e. represented by the equation:

$$f ( d ) = kd + c$$

wherein k and c are constants. The value of the constant k and c can be determined experimentally.

### EXAMPLES

### Example 1—Preparation of an Aqueous Ultrasound Coupling Agent Concentrate

A concentrate of aqueous ultrasound coupling agent was formulated as an emulsion. The concentrate contained at least one triglyceride, at least one phospholipid, water, and glycerol. The concentrate was then diluted to the appropriate concentration with water. The concentrate did not include a stabilizer (e.g., no polysorbate 80 was present) or saline (i.e., no sodium chloride was present). The pH was adjusted to about 6.5 to about 8.5 with NaOH. The table below provides an exemplary concentrate formulation.

TABLE A

| | Amount in concentrate | Amount in diluted |
|---|---|---|
| Ingredient | (g/L) | formulation (% w/v) |
| Triglyceride (e.g., | 70 | 7 |
| soybean oil) | | |
| Glycerol | 7.7 | 0.8 |
| Phospholipids | 4.2 | 0.4 |
| water | | 91.8 |

The droplet size, osmolality, and pH of the emulsion were measured as described below.

Samples were stored at room temperature, with no direct exposure to sunlight. Visual analysis was done by observation of the syringe prior to extraction for further analysis, without any agitation of the samples which might cause redispersion or temporary mixing. For every measurement point, 5 ml of the emulsion was squeezed from each syringe after visual analysis. The 5 ml aliquots were subsequently used for all analyses of a specific time point. The autoclave (CertoClav® CVEL 12 L) temperature was controlled manually via a pressure gauge, and the duration of each cycle was controlled via a timer. The conditions used were 20 minutes at 121° C.

pH measurements were collected using a Mettler Toledo SevenEasy pH-meter calibrated with AVS TITRINORM Buffer solution pH 4 and AVS TITRINORM Buffer solution pH 7, and controlled with AVS TITRJNORM Buffer solution pH 5 as part of a daily routine.

Dynamic light scattering measurements were collected using a Malvern Zetasizer Nano Series Nano-ZS. Here, the sample was diluted by a factor of 100 in DI water immediately prior to analysis. For size control and calibration, a Nanosphere size Standard 203±5 nm was used. This is made fresh and run every day the measurement was done. Average droplet size reported from ZetaSizer measurements is the Z-average diameter.

The microscope used is an Olympos BX43 equipped with an Olympus XM10 camera with pre-calibrated measurements (embedded in images). Osmolality measurements were made using an OSMOMAT 030-D-D3P from GonoTec, Ser. No. 08 02 22. Here, 50 microliter of the sample was pipetted into the measuring vessel, making sure to avoid any formation of air bubbles. Prior to sample analysis, the instrument was calibrated with a GonoTec calibration standard with an expected value of 300 mOsmol/kg NaCl/H20 (acceptable range 298-302 mOsmol/kg). The calibration standard was run in triplicate.

### Example 2—Evaluation of an Aqueous Ultrasound Coupling Agent for Ultrasound-Guided Imaging of the Brain

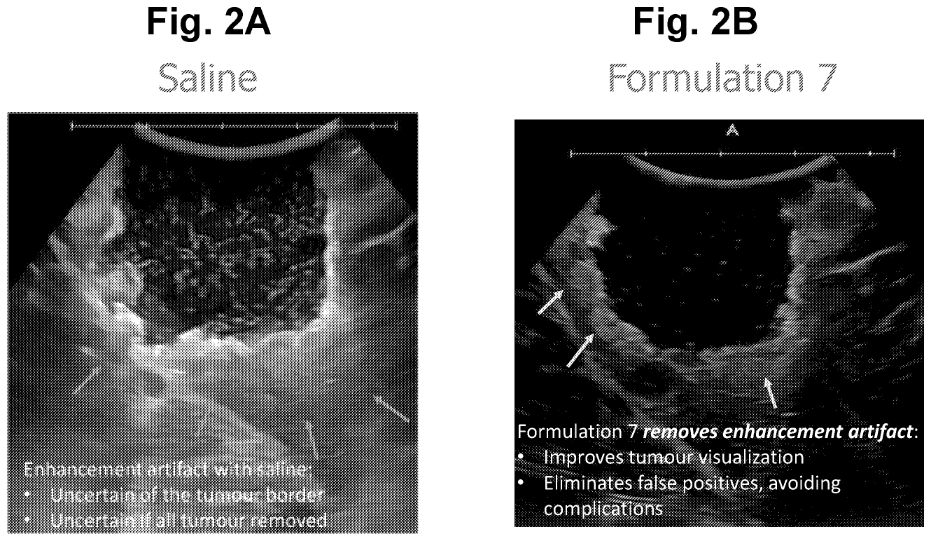

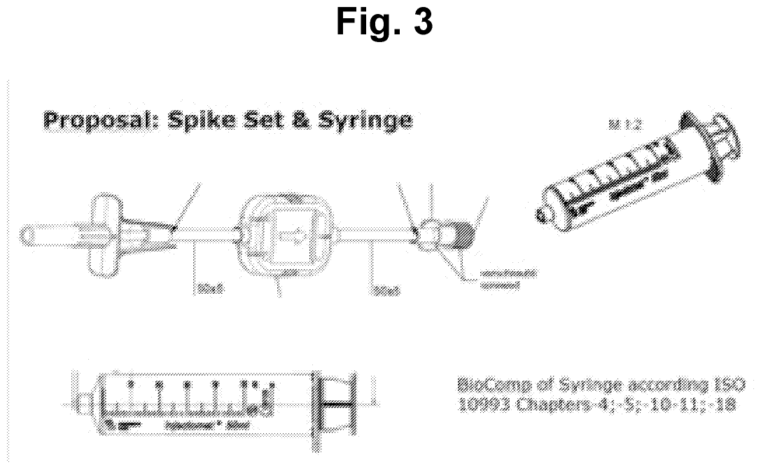

Aqueous ultrasound coupling agents are critical for ultrasound-guided resection of brain tumors. In the absence of an aqueous ultrasound coupling agent (e.g., in the presence of saline alone), ultrasound images are difficult to interpret due to the presence of enhancement artefacts that make it difficult to determine if residual tumor exists in the cavity. Developing aqueous ultrasound coupling agents for the brain is challenging because they must have substantially similar acoustic properties to cerebrospinal fluid and be safe. The ideal aqueous ultrasound coupling agent for the brain allows a neurosurgeon to precisely identify and remove tumor cells. One aqueous ultrasound coupling agent that has been used for this purpose is Formulation 7 of Table B, below. In comparison to saline, the use of Formulation 7 as an aqueous ultrasound coupling agent results in removal of enhancement artefacts and improvement of tumor visualization. (compare FIG. 2A (use of saline) to FIG. 2B (use of Formulation 7 as an aqueous ultrasound coupling agent). While Formulation 7 has ideal acoustic properties for imaging and thus allows neurosurgeons to precisely identify and remove tumor cells, safety concerns were identified with this formulation due to the presence of polysorbate 80 (PS80) in the formulation. PS80 is a surfactant that is used to stabilize fat emulsions and may unsafely alter lipid content in the brain.

The development of alternative formulations that exhibit acoustic properties that are equivalent or better than those of Formulation 7, but also that lack PS80, is challenging. Because PS80 was used as a stabilizer, to stabilize droplets in the emulsion and create a homogeneous solution, removal of PS80 was expected to cause triglycerides and phospholipids in the composition to come out of solution (phase separate), which could make the aqueous ultrasound coupling agent ineffective for tumor imaging.

In the prior versions of the ultrasound coupling agent, sodium chloride was used to modify osmolality of the coupling agent such that it was isotonic to cerebral spinal fluid. However, if PS80 was removed to address the safety issues, the sodium chloride would have destabilized the emulsion, causing the triglycerides and phospholipids to phase separate. The resulting composition would not be suitable for use as an ultrasound coupling agent. Further, removing sodium chloride from the formulation would have changed the osmolality of the coupling agent such that it was no longer isotonic to cerebral spinal fluid. Contrast agents must be isotonic with their environment to prevent them from affecting surrounding cells. For instance, if the contrast agent was hypertonic with its environment, water would be drawn out of cells and cells would collapse. This would be a serious safety risk.

The inventor herein has unexpectedly discovered that formulations lacking both PS80 and sodium chloride can effectively serve as aqueous ultrasound coupling agents and can be used to accurately visualize a brain tissue-mimicking phantom (Cirs Multipurpose Phantom). The advantage of these formulations is that they lack PS80 and thus do not present safety concerns.

The formulations of Table B were evaluated for imaging the tissue mimicking phantom:

TABLE B

| Formulation | Components | D[4, 3] (μm) |
|---|---|---|
| 1 | 0.9% w/v NaCl | |
| 2 | 5% w/v triglycerides | 0.401 |
| | 4.2 g/L phospholipids (i.e., egg yolk | |
| | phospholipids) | |
| | 23.7 g/L glycerol | |
| 3 | 7% w/v triglycerides | 0.389 |
| | 4.2 g/L phospholipids (i.e., egg yolk | |
| | phospholipids) | |
| | 23.7 g/L glycerol | |
| 4 | 9% w/v triglycerides | 0.402 |
| | 4.2 g/L phospholipids (i.e., egg yolk | |
| | phospholipids) | |
| | 23.7 g/L glycerol | |
| 5 | 7% w/v triglycerides | 0.269 |
| | 4.2 g/L phospholipids (i.e., egg yolk | |
| | phospholipids) | |
| | 23.7 g/L glycerol | |
| 6 | 7% w/v triglycerides | 0.498 |
| | 4.2 g/L phospholipids (i.e., egg yolk | |
| | phospholipids) | |
| | 23.7 g/L glycerol | |
| 7 | 7% w/v triglycerides | 0.285 |
| | 4.2 g/L phospholipids (i.e., egg yolk | |
| | phospholipids) | |
| | 7.7 g/L glycerol | |
| | 0.65% PS80 | |
| | 5.85 g/L NaCl (0.59%) | |

The phantom had an attenuation coefficient of 0.5 dB/cm-MHz±0.07, a speed of sound of 1540 m/s+10 m/s, and a scatter or relative contrast of −15 dB to +15 dB. The phantom has tubular hyperechoic and hypoechoic structures of between 3 cm and 4 cm. The phantom also contained a cubic cavity of about 2 cm length, 1 cm width, and 2 cm depth, in premade tubular structures.

The cubic cavity of the phantom was filled with one of the formulations from Table B and imaged on an ultrasound device (a BK5000 using Autogain or Fixed Gain). A neurosurgeon evaluated the (1) enhancement artifacts, (2) visual CNR, (3) overall image quality, and (4) similarity of Formulations 1-6 to Formulation 7 on a scale of 1 to 5, with a higher score being most desirable for imaging. Table C shows the scores for each Formulation using a BK5000 ultrasound device.

TABLE C

| | Formulation | | | | | | |
|---|---|---|---|---|---|---|---|
| | 1 | 2 | 3 | 4 | 5 | 6 | 7 |
| Enhancement Artefacts | 2/1 | 4/4 | 5/5 | 4/4 | 5/5 | 4/4 | 4/5 |
| Visual CNR | 2/2 | 3/3 | 4/5 | 4/4 | 4/4 | 5/4 | 4/4 |
| Overall Image Quality | 2/2 | 4/4 | 4/4 | 3/4 | 5/5 | 4/4 | 5/4 |
| Similarity to | 1/1 | 5/5 | 5/5 | 5/5 | 5/5 | 5/5 | 5/5 |
| Formulation 7 | | | | | | | |
| Total Score | 7 | 16 | 18 | 16 | 19 | 18 | 18 |
| AutoGain | | | | | | | |
| Total Score | 6 | 16 | 19 | 17 | 19 | 17 | 18 |
| Fixed Gain | | | | | | | |
| Average Score | 6.5 | 16 | 18.5 | 16.5 | 19 | 17.5 | 18 |

Each subscore was determined using autogain and fixed gain.
The subscores are represented in the table as autogain score/fixed gain score.

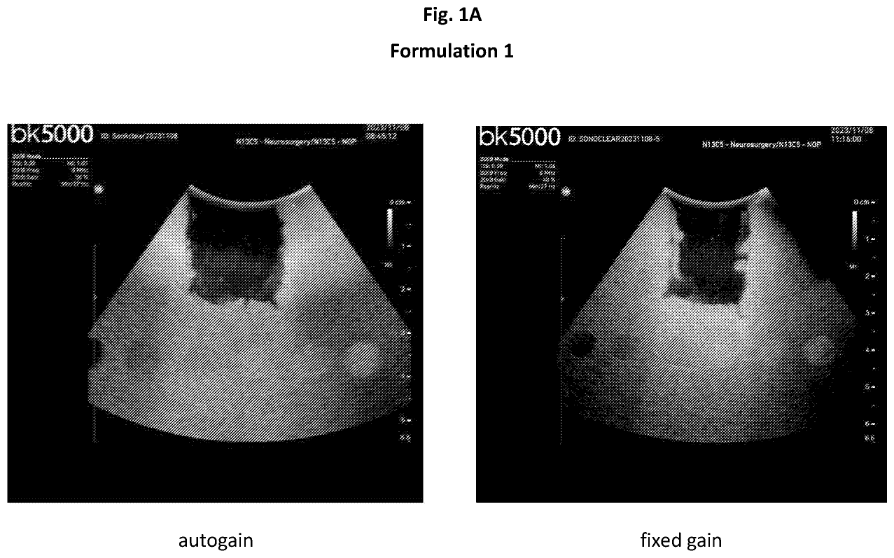

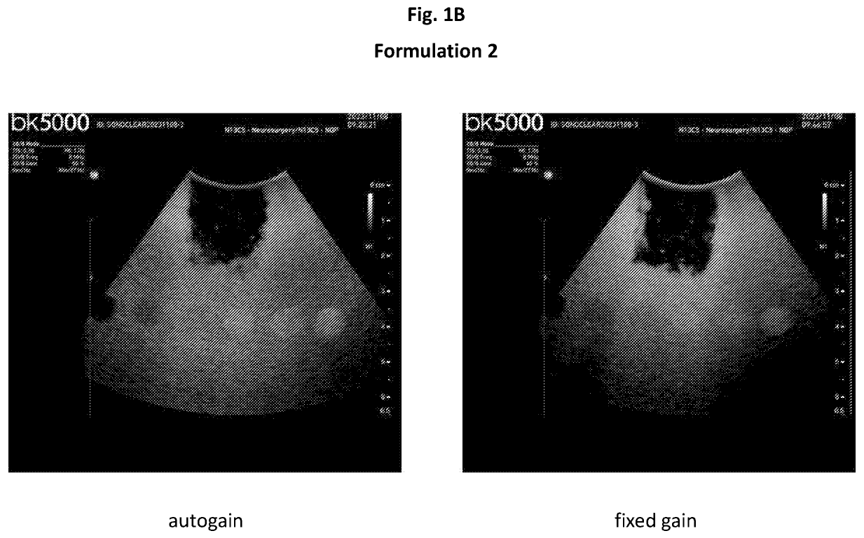

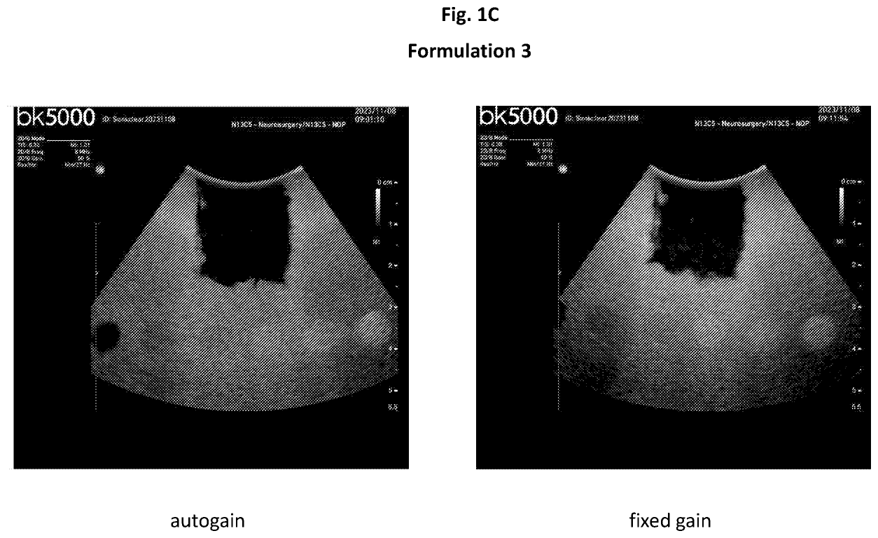

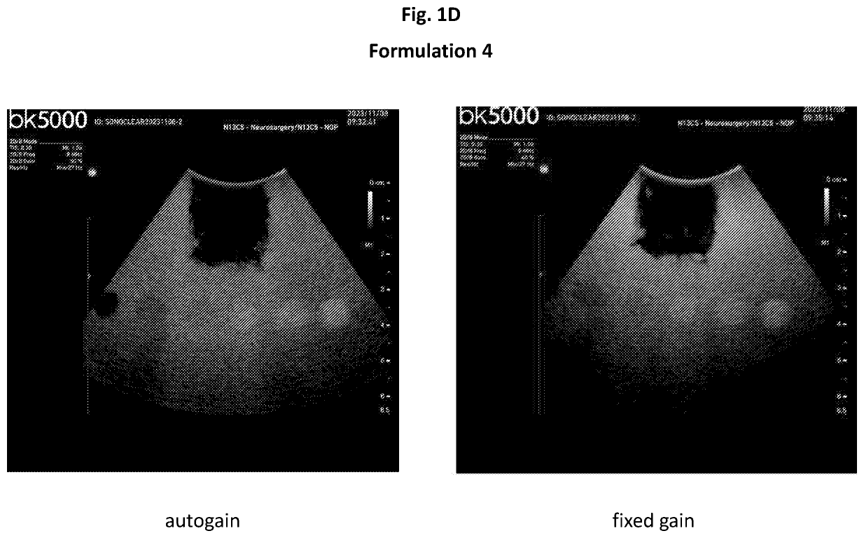

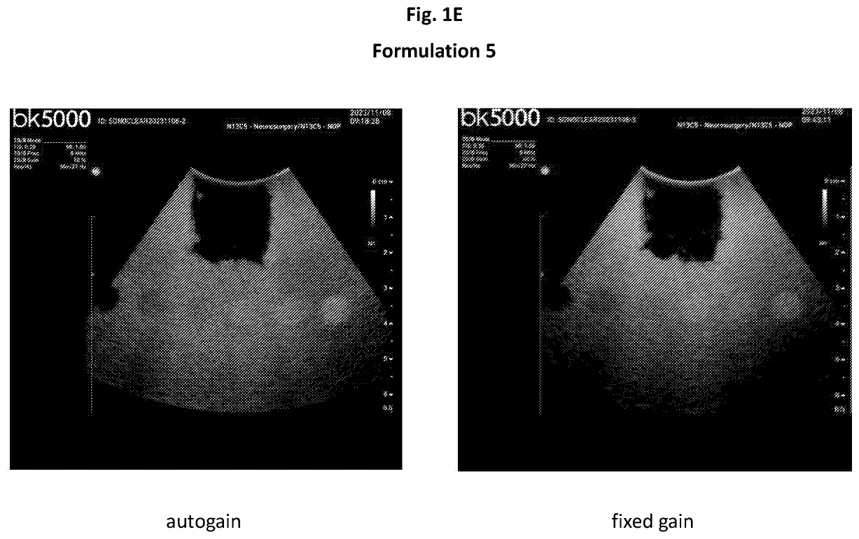

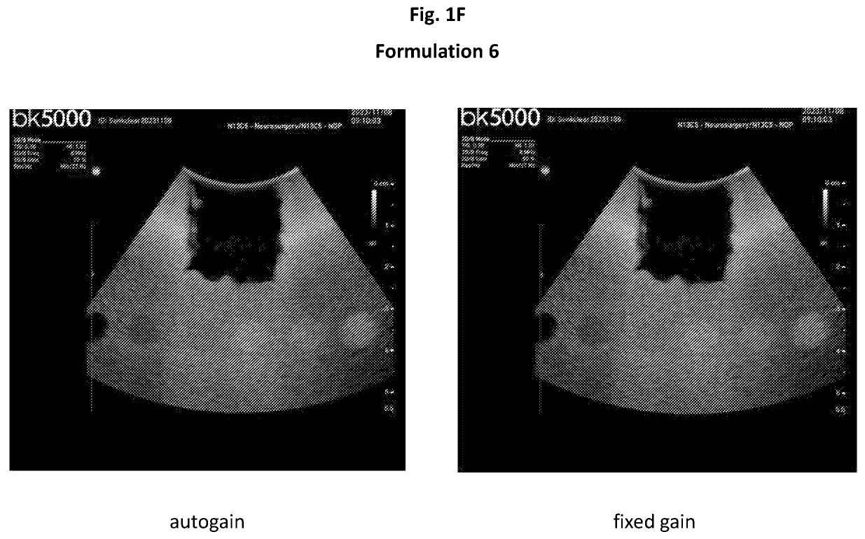

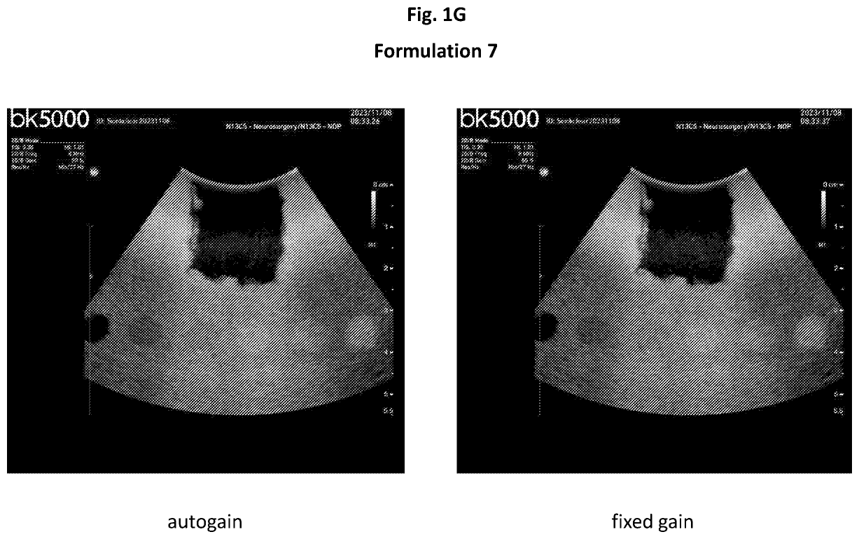

Formulations that contained 7% w/v triglycerides (Formulations 3 (FIG. 1C), 5 (FIG. 1E), and 6 (FIG. 1F)) were superior aqueous ultrasound coupling agents to formulations that contained 5% w/v (Formulation 2, FIG. 1B) or 9% w/v triglycerides (Formulation 4, FIG. 1D). Surprisingly, formulations that lacked salt and stabilizing agent were substantially similar to or improved compared to Formulation 7, which contained salt (i.e., NaCl) and stabilizing agent (i.e., PS80). Also surprising was the fact that Formulations 3, 5, and 6 contained 207% more glycerol than Formulation 7 but still were suitable aqueous ultrasound coupling agents.

### Example 3—Ultrasound Fluid Characteristics of Aqueous Ultrasound Coupling Agents

The particle size of an aqueous ultrasound coupling agent must be under 5 μm to safely administer an aqueous ultrasound coupling agent via intravenous administration. The osmolality of an aqueous ultrasound coupling agent must be from 270-330 mOsm/L to ensure that the aqueous ultrasound coupling agent is isotonic with cerebral spinal fluid. The pH of the aqueous ultrasound coupling agent should be from about 6.5 to about 8.5. Multiple aqueous ultrasound coupling agents were evaluated to determine if they exhibited ideal particle size, osmolality, and pH.

Initial formulations were generated by adding glycerol to three different lipid emulsion stock solutions. Each of the lipid emulsion stock solutions contains different sources of triglycerides. For example, the triglyceride source for Lipid Emulsion 1 is soybean oil. The triglyceride source for Emulsion 2 is medium-chain triglcyerides, olive oil, and fish oil, and the triglyceride source for Emulsion 3 is highly refined fish oil. Each of the lipid emulsion stock solutions also contains a different source of phospholipid. The formulations of each lipid emulsion stock solutions are in Table D.

TABLE D

| | Lipid | Lipid | Lipid |
|---|---|---|---|
| | Emulsion 1 | Emulsion 2 | Emulsion 3 |
| | (Intralipid ®) | (SMOFlipid ®) | (Omegaven ®) |
| Ingredient | 20% [g/L] | 20% [g/L] | 10% [g/L] |
| Water for Injections | to 1 L | to 1 L | to 1 L |
| Soybean oil | 200.0 | 60.0 | — |
| Egg lecithin FKAB | 12.0 | — | — |
| Egg lecithin PL90 | — | 12.0 | 12.0 |
| Glycerol | 22.0 | 25.0 | 25.0 |
| Sodium hydroxide | q.s. | q.s. | q.s. |
| solution 1M | | | |
| Medium-chain | — | 60.0 | — |
| triglycerides | | | |
| Refined olive oil | — | 50.0 | — |
| Purified fish oil | — | 30.0 | — |
| Highly refined fish oil | — | — | 100.0 |
| dl-α-Tocopherol | — | 0.135 | — |
| Sodium oleate | — | 0.3 | 0.3 |
| Nitrogen | as required | as required | as required |

The formulations of Table D were diluted to 5%, 7%, or 9% w/v triglyceride and a various amounts of glycerol was added to each formulation. Table E shows the pH and osmolality of each formulation with varying amounts of glycerol.

TABLE E

| | | | NaOH | Added | | |
|---|---|---|---|---|---|---|
| | Triglyceride | | 1M in | Glycerol | pH- | |
| Batch no. | (% w/v) | Diluted from | mL/L | in g/L | value | Osmolality |
| HH2338007 | 5% | Lipid Emulsion 2 | 0 | 8.00 | 7.2 | 274 |
| HH2338007 | 5% | Lipid Emulsion 2 | 0 | 9.50 | 7.3 | 309 |
| HH2338007 | 5% | Lipid Emulsion 2 | 0 | 11.00 | 7.3 | 348 |
| HH2338008 | 7% | Lipid Emulsion 2 | 0 | 6.00 | 7.5 | 264 |
| HH2338008 | 7% | Lipid Emulsion 2 | 0 | 7.50 | 7.5 | 302 |
| HH2338008 | 7% | Lipid Emulsion 2 | 0 | 9.00 | 7.5 | 340 |
| HH2338009 | 9% | Lipid Emulsion 2 | 0 | 4.50 | 7.6 | 265 |
| HH2338009 | 9% | Lipid Emulsion 2 | 0 | 6.01 | 7.6 | 306 |
| HH2338009 | 9% | Lipid Emulsion 2 | 0 | 7.50 | 7.6 | 343 |
| HH2338010 | 5% | Lipid Emulsion 3 | 0 | 4.50 | 7.5 | 266 |
| HH2338010 | 5% | Lipid Emulsion 3 | 0 | 6.00 | 7.6 | 304 |
| HH2338010 | 5% | Lipid Emulsion 3 | 0 | 7.50 | 7.6 | 340 |
| HH2338011 | 7% | Lipid Emulsion 3 | 0 | 1.50 | 7.7 | 263 |
| HH2338011 | 7% | Lipid Emulsion 3 | 0 | 2.99 | 7.7 | 305 |
| HH2338011 | 7% | Lipid Emulsion 3 | 0 | 4.50 | 7.8 | 342 |
| HH2338011 | 9% | Lipid Emulsion 3 | 0 | 0 | 7.8 | 304 |
| HH2338012 | 5% | Lipid Emulsion 1 | 0 | 8.50 | 6.9 | 272 |
| HH2338012 | 5% | Lipid Emulsion 1 | 0 | 10.00 | 6.9 | 311 |
| HH2338012 | 5% | Lipid Emulsion 1 | 0 | 11.51 | 6.9 | 349 |
| HH2338013 | 7% | Lipid Emulsion 1 | 0 | 6.50 | 7.0 | 261 |
| HH2338013 | 7% | Lipid Emulsion 1 | 0 | 8.00 | 7.0 | 296 |
| HH2338013 | 7% | Lipid Emulsion 1 | 0 | 9.50 | 7.0 | 336 |
| HH2338014 | 9% | Lipid Emulsion 1 | 0 | 5.05 | 7.2 | 259 |
| HH2338014 | 9% | Lipid Emulsion 1 | 0 | 6.55 | 7.2 | 294 |
| HH2338014 | 9% | Lipid Emulsion 1 | 0 | 8.05 | 7.2 | 336 |

Formulations with osmolalities from about 290 mOsm/kg-311 mOsm/kg were then generated, and the oil droplet size was characterized. Table F shows the Mastersizer 100%≤x μm value and the D [4,3] value for each formulation. The Mastersizer 100%≤x μm value (“Mastersizer”) describes the oil droplet diameter, where 100% of the oil droplets are smaller. The D [4,3] is the volume weighted mean droplet size.

TABLE F

| | | NaOH | Added | | | | |
|---|---|---|---|---|---|---|---|
| | | 1M in | Glycerol | pH- | Osmolality | Mastersizer | D [4, 3] |
| Batch no. | Sample | mL/L | in g/L | value | mOsm/kg | (μm) | (μm) |
| HH2341017 | Intralipid 5% | 0.065 | 20.01 | 7.2 | 311 | 0.955 | 0.401 |
| HH2341018 | Intralipid 7% | 0.100 | 16.00 | 7.5 | 299 | 0.955 | 0.389 |
| HH2341019 | Intralipid 9% | 0.140 | 13.10 | 7.7 | 298 | 0.955 | 0.402 |
| HH2341020 | SMOF 5% | 0.060 | 19.00 | 7.9 | 308 | 0.955 | 0.380 |
| HH2341021 | SMOF 7% | 0.080 | 15.00 | 7.9 | 301 | 0.955 | 0.382 |
| HH2341022 | SMOF 9% | 0.130 | 12.00 | 8.0 | 303 | 0.955 | 0.379 |
| HH2341023 | Omegaven | 0 | 12.00 | 8.0 | 301 | 0.724 | 0.271 |
| | 5% | | | | | | |
| HH2341024 | Omegaven | 0 | 6.00 | 7.9 | 303 | 0.724 | 0.278 |
| | 7% | | | | | | |
| HH2341025 | Omegaven | 0 | 0 | 8.0 | 300 | 0.724 | 0.276 |
| | 9% | | | | | | |
| HH2341026 | Intralipid 7% | 0.300 | 23.70 | 6.9 | 294 | 1.905 | 0.498 |
| | high droplet | | | | | | |
| | size | | | | | | |
| HH2341027 | Intralipid 7% | 0.300 | 23.70 | 6.9 | 297 | 0.832 | 0.269 |
| | low droplet | | | | | | |
| | size | | | | | | |

Each of the formulations described above had both optimal osmolality and oil droplet size distribution for imaging brain tumors.

### NUMBERED EMBODIMENTS OF THE DISCLOSURE

Notwithstanding the appended claims, the disclosure sets for the following numbered embodiments:

1. An aqueous ultrasound coupling agent consisting of:
a) from about 60.0 to about 80.0 g/L of at least one triglyceride;
b) from about 3.2 to about 5.2 g/L of at least one phospholipid;
c) water;
d) pH buffer; and
e) glycerol;
   wherein the ultrasound coupling agent has an attenuation constant from about 0.15 dB/(MHz*cm) to about 0.60 dB/(MHz*cm) or from about 0.002 to about 1.0 dB/(MHz*cm).

2. An aqueous ultrasound coupling agent consisting of:
a) at least one triglyceride;
b) at least one phospholipid;
c) water, and
d) pH buffer; and
e) glycerol;
wherein the ultrasound coupling agent has an attenuation constant from about 0.15 dB/(MHz*cm) to about 0.60 dB/(MHz*cm) or from about 0.002 to about 1.0 dB/(MHz*cm); and wherein the ratio of the at least one triglyceride to the at least one phospholipid is from about 13:1 to about 20:1 by weight.

3. An aqueous ultrasound coupling agent consisting of:
a) at least one triglyceride, wherein the at least one triglyceride is present at about 7% w/V;
b) at least one phospholipid, wherein the at least one phospholipid is present at about 0.4% w/v;
c) water, wherein the water is present at about 92% w/v;
d) pH buffer; and
e) glycerol.

4. An aqueous ultrasound coupling agent comprising:
a) from about 60.0 g/L to about 80.0 g/L of at least one triglyceride;
b) from about 3.2 g/L to 5.2 g/L of at least one phospholipid,
wherein the coupling agent does not include a stabilizer and does not include sodium chloride, and
wherein the ultrasound coupling agent has an attenuation constant from about 0.15 dB/(MHz*cm) to about 0.60 dB/(MHz*cm) or from about 0.002 to about 1.0 dB/(MHz*cm).

5. An aqueous ultrasound coupling agent comprising:
a) at least one triglyceride; and
b) at least one phospholipid;
wherein the coupling agent does not include a stabilizer and does not include sodium chloride;
wherein the ultrasound coupling agent has an attenuation constant from about 0.15 dB/(MHz*cm) to about 0.60 dB/(MHz*cm) or from about 0.002 to about 1.0 dB/(MHz*cm); and
wherein the ratio of the at least one triglyceride to the at least one phospholipid is from about 13:1 to about 20:1 by weight.

6. An aqueous ultrasound coupling agent comprising:
a) at least one triglyceride, wherein the at least one triglyceride is present at about 7% w/v; and
b) at least one phospholipid, wherein the at least one phospholipid is present at about 0.4% w/V;
wherein the coupling agent does not include a stabilizer and does not include sodium chloride; and
wherein the ultrasound coupling agent has an attenuation constant from about 0.15 dB/(MHz*cm) to about 0.60 dB/(MHz*cm) or from about 0.002 to about 1.0 dB/(MHz*cm).

7. The aqueous ultrasound coupling agent of embodiment 1, 2, 4, and 5, comprising about 70 g/L of at least one triglyceride.

8. The aqueous ultrasound coupling agent of any one of embodiments 1-7, wherein the at least one triglyceride is from one or more of soybean oil, olive oil, or fish oil.

9. The aqueous ultrasound coupling ag1-ent of any one of embodiments 1-8, wherein the at least one triglyceride is from soybean oil.

10. The aqueous ultrasound coupling agent of any one of embodiments 4-9, comprising at least one humectant.

11. The aqueous ultrasound coupling agent of embodiment 10, wherein the at least one humectant is glycerol.

12. The aqueous ultrasound coupling agent of any one of embodiments 4-11, wherein the aqueous ultrasound coupling agent does not comprise PS80.

13. The aqueous ultrasound coupling agent of any one of embodiments 1-12, wherein the pH is from about 6.5 to about 8.5.

14. The aqueous ultrasound coupling agent of any one of embodiments 1-12, wherein the pH is about 7.3

15. The aqueous ultrasound coupling agent of any one of embodiments 1-14, wherein the osmolality is from 270-330 mOsm/L.

16. The aqueous ultrasound coupling agent of any one of embodiments 1-15, wherein the coupling agent has an average droplet size of about 285 nm±20%, wherein the average droplet size is measured by dynamic light scattering.

17. The aqueous ultrasound coupling agent of any one of embodiments 1-15, wherein the shelf life of the aqueous ultrasound coupling agent is at least about 10 hours.

18. The aqueous ultrasound coupling agent of any one of embodiments 1-17, wherein the ratio of the at least one triglyceride to the at least one phospholipid is about 16.7:1 by weight.

19. A method of obtaining an ultrasound image of an organ comprising
i) contacting the organ with an ultrasound coupling agent of any one of embodiments 1-18;
ii) obtaining an ultra sound image of the organ,
wherein the coupling agent does not include a stabilizer and does not include sodium chloride, and
wherein the ultrasound coupling agent has an attenuation constant from about 0.15 dB/(MHz*cm) to about 0.60 dB/(MHz*cm) or from about 0.002 to about 1.0 dB/(MHz*cm).

20. The method of embodiment 19, wherein the organ is a brain.

### ADDITIONAL EMBODIMENTS

1. An aqueous ultrasound coupling agent consisting essentially of:
(a) from about 60.0 g/L to about 80.0 g/L of at least one triglyceride;
(b) from about 3.2 g/L to about 5.2 g/L of at least one phospholipid;
(c) water;
(d) pH buffer; and
(e) glycerol.

2. An aqueous ultrasound coupling agent consisting essentially of:
(a) at least one triglyceride;
(b) at least one phospholipid;
(c) water,
(d) pH buffer; and
(e) glycerol; and
wherein the ratio of the at least one triglyceride to the at least one phospholipid is from about 13:1 to about 20:1 by weight.

3. An aqueous ultrasound coupling agent consisting essentially of:
(a) between 5% w/v and 9% w/v of at least one triglyceride;
(b) from about 0.2% w/v to about 1.2% w/v of at least one phospholipid;
(c) water;
(d) pH buffer; and
(e) glycerol.

4. An aqueous ultrasound coupling agent consisting essentially of:
(a) between 5% w/v and 9% w/v of at least one triglyceride;
(b) at least one phospholipid, wherein the at least one phospholipid is present at about 0.42% w/V;
(c) water;
(d) pH buffer; and
(e) glycerol.

5. An aqueous ultrasound coupling agent consisting essentially of:
(a) between 5% w/v and 9% w/v of at least one triglyceride;
(b) at least one phospholipid;
(c) water;
(d) pH buffer; and
(e) glycerol.

6. An aqueous ultrasound coupling agent consisting essentially of:
(a) between 5% w/v and 9% w/v of at least one triglyceride;
(b) from about 0.2% w/v to about 1.2% w/v of at least one phospholipid;
(c) water;
(d) pH buffer; and
(e) from about 21 g/L to about 26 g/L of glycerol.

7. An aqueous ultrasound coupling agent consisting essentially of:
(a) about 7% w/v of a triglyceride;
(b) about 4.2 g/L of a phospholipid;
(c) about 23.7 g/L of glycerol;
(d) water; and
(e) a pH buffer.

8. An aqueous ultrasound coupling agent consisting essentially of:
(a) from about 60.0 to about 80.0 g/L of at least one triglyceride;
(b) from about 3.2 to about 5.2 g/L of lecithin;
(c) water;
(d) pH buffer; and
(e) glycerol.

9. An aqueous ultrasound coupling agent consisting essentially of:
(a) at least one triglyceride;
(b) lecithin;
(c) water,
(d) pH buffer; and
(e) glycerol; and
wherein the ratio of the at least one triglyceride to the at least one phospholipid is from about 13:1 to about 20:1 by weight.

10. An aqueous ultrasound coupling agent consisting essentially of:
(a) between 5% w/v and 9% w/v of at least one triglyceride;
(b) from about 0.2% w/v to about 1.2% w/v of lecithin;
(c) water;
(d) pH buffer; and
(e) glycerol.

11. An aqueous ultrasound coupling agent consisting essentially of:
(a) between 5% w/v and 9% w/v of at least one triglyceride;
(b) about 0.42% w/v lecithin;
(c) water;
(d) pH buffer; and
(e) glycerol.

12. An aqueous ultrasound coupling agent consisting essentially of:
(a) between 5% w/v and 9% w/v of at least one triglyceride;
(b) lecithin;
(c) water;
(d) pH buffer; and
(e) glycerol.

13. An aqueous ultrasound coupling agent consisting essentially of:
(a) between 5% w/v and 9% w/v of at least one triglyceride;
(b) from about 0.2% w/v to about 1.2% w/v of lecithin;
(c) water;
(d) pH buffer; and
(e) from about 21 g/L to about 26 g/L of glycerol.

14. An aqueous ultrasound coupling agent consisting essentially of:
(a) 7% w/v of a triglyceride;
(b) about 4.2 g/L lecithin;
(c) about 23.7 g/L of glycerol;
(d) water; and
(e) a pH buffer.

15. An aqueous ultrasound coupling agent comprising:
(a) from about 60.0 to about 80.0 g/L of at least one triglyceride;
(b) from about 3.2 to about 5.2 g/L of at least one phospholipid;
(c) water;
(d) pH buffer; and
(e) glycerol;
wherein the coupling agent does not include a stabilizer and does not include sodium chloride.

16. An aqueous ultrasound coupling agent comprising:
(a) at least one triglyceride;
(b) at least one phospholipid;
(c) water;
(d) pH buffer; and
(e) glycerol; and
wherein the ratio of the at least one triglyceride to the at least one phospholipid is from about 13:1 to about 20:1 by weight;
wherein the coupling agent does not include a stabilizer and does not include sodium chloride.

17. An aqueous ultrasound coupling agent comprising:
(a) between 5% w/v and 9% w/v of at least one triglyceride;
(b) from about 0.2% w/v to about 1.2% w/v of at least one phospholipid;
(c) water;
(d) pH buffer; and
(e) glycerol;
wherein the coupling agent does not include a stabilizer and does not include sodium chloride.

18. An aqueous ultrasound coupling agent comprising:
(a) between 5% w/v and 9% w/v of at least one triglyceride;
(b) at least one phospholipid, wherein the at least one phospholipid is present at about 0.42% w/V;
(c) water;
(d) pH buffer; and
(e) glycerol;
wherein the coupling agent does not include a stabilizer and does not include sodium chloride.

19. An aqueous ultrasound coupling agent comprising:
(a) between 5% w/v and 9% w/v of at least one triglyceride;
(b) at least one phospholipid;
(c) water;
(d) pH buffer; and
(e) glycerol;
wherein the coupling agent does not include a stabilizer and does not include sodium chloride.

20. An aqueous ultrasound coupling agent comprising:
(a) between 5% w/v and 9% w/v of at least one triglyceride;
(b) from about 0.2% w/v to about 1.2% w/v of at least one phospholipid;
(c) water;
(d) pH buffer; and
(e) from about 21 g/L to about 26 g/L of glycerol;
wherein the coupling agent does not include a stabilizer and does not include sodium chloride.

21. An aqueous ultrasound coupling agent comprising:
(a) about 7% w/v of a triglyceride;
(b) about 4.2 g/L of a phospholipid;
(c) about 23.7 g/L of glycerol;
(d) water; and
(e) a pH buffer;
wherein the coupling agent does not include a stabilizer and does not include sodium chloride.

22. An aqueous ultrasound coupling agent comprising:
(a) from about 60.0 to about 80.0 g/L of at least one triglyceride;
(b) from about 3.2 to about 5.2 g/L of lecithin;
(c) water;
(d) pH buffer; and
(e) glycerol;
wherein the coupling agent does not include a stabilizer and does not include sodium chloride.

23. An aqueous ultrasound coupling agent comprising:
(a) at least one triglyceride;
(b) lecithin;
(c) water,
(d) pH buffer; and
(e) glycerol; and wherein the ratio of the at least one triglyceride to the at least one phospholipid is from about 13:1 to about 20:1 by weight;
wherein the coupling agent does not include a stabilizer and does not include sodium chloride.

24. An aqueous ultrasound coupling agent comprising:
(a) between 5% w/v and 9% w/v of at least one triglyceride;
(b) from about 0.2% w/v to about 1.2% w/v of lecithin;
(c) water;
(d) pH buffer; and
(e) glycerol;
wherein the coupling agent does not include a stabilizer and does not include sodium chloride.

25. An aqueous ultrasound coupling agent comprising:
(a) between 5% w/v and 9% w/v of at least one triglyceride;
(b) about 0.42% w/v lecithin;
(c) water;
(d) pH buffer; and
(e) glycerol;
wherein the coupling agent does not include a stabilizer and does not include sodium chloride.

26. An aqueous ultrasound coupling agent comprising:
(a) between 5% w/v and 9% w/v of at least one triglyceride;
(b) lecithin;
(c) water;
(d) pH buffer; and
(e) glycerol;
wherein the coupling agent does not include a stabilizer and does not include sodium chloride.

27. An aqueous ultrasound coupling agent comprising:
(a) between 5% w/v and 9% w/v of triglyceride;
(b) from about 0.2% w/v to about 1.2% w/v of at least one lecithin;
(c) water;
(d) pH buffer; and
(e) from about 21 g/L to about 26 g/L of glycerol;
wherein the coupling agent does not include a stabilizer and does not include sodium chloride 28. An aqueous ultrasound coupling agent comprising:
(a) about 7% w/v of a triglyceride;
(b) about 4.2 g/L lecithin;
(c) about 23.7 g/L of glycerol;
(d) water; and
(e) a pH buffer;
wherein the coupling agent does not include a stabilizer and does not include sodium chloride.

29. An aqueous ultrasound coupling agent comprising:
(a) about 7% w/v of a triglyceride;
(b) about 4.2 g/L of at least one phospholipid;
(c) about 23.7 g/L of glycerol;
(d) water; and
(e) a pH buffer.

30. An aqueous ultrasound coupling agent comprising:
(a) about 7% w/v of a triglyceride;
(b) about 4.2 g/L lecithin;
(c) about 23.7 g/L of glycerol;
(d) water; and
(e) a pH buffer.

31. The aqueous ultrasound coupling agent of any one of embodiments 15-28, wherein the stabilizer is polysorbate 80 (PS80).

32. The aqueous ultrasound coupling agent of any one of embodiments 1-31, wherein the ultrasound coupling agent has an attenuation coefficient of from about 0.15 dB/(MHz*cm) to about 0.60 dB/(MHz*cm).

33. The aqueous ultrasound coupling agent of any one of embodiments 1-31, wherein the ultrasound coupling agent has an attenuation coefficient of from about 0.002 dB/(MHz*cm) to about 1.0 dB/(MHz*cm).

34. The aqueous ultrasound coupling agent of any one of embodiments 1-31, wherein the ultrasound coupling agent has an attenuation coefficient from about 0.05 dB/(MHz*cm) to about 15 dB/(MHz*cm).

35. The aqueous ultrasound coupling agent of any one of embodiments 1-31, wherein the ultrasound coupling agent has an attenuation coefficient from about 0.05 dB/(MHz*cm) to about 1.8 dB/(MHz*cm).

36. The aqueous ultrasound coupling agent of any one of embodiments 1-31, wherein the ultrasound coupling agent has an attenuation coefficient that is from about 0.5 dB/(MHz*cm) to about 1.5 dB/(MHz*cm).

37. The aqueous ultrasound coupling agent of any one of embodiments 1-31, wherein the ultrasound coupling agent has an attenuation coefficient that is from about 3 dB/(MHz*cm) to about 15 dB/(MHz*cm).

38. The aqueous ultrasound coupling agent of any one of embodiments 1-31, wherein the ultrasound coupling agent has an attenuation coefficient that is from about 0.1 dB/(MHz*cm) to about 0.5 dB/(MHz*cm).

39. The aqueous ultrasound coupling agent of any one of embodiments 1-31, wherein the ultrasound coupling agent has an attenuation coefficient that is from about 0.1 dB/(MHz*cm) to about 1.5 dB/(MHz*cm).

40. The aqueous ultrasound coupling agent of any one of embodiments 1-31, wherein the ultrasound coupling agent has an attenuation coefficient that is substantially similar to an attenuation coefficient of bone, brain, lung, liver, kidney, heart, cerebrospinal fluid, a soft tissue, a bladder, or a colon.

41. The aqueous ultrasound coupling agent of any one of embodiments 1-40, wherein the at least one triglyceride is from one or more of olive oil, coconut oil, palm oil, canola oil, soybean oil, sunflower oil, corn oil, peanut oil, safflower oil, cottonseed oil, sesame oil, flaxseed oil, walnut oil, avocado oil, rice bran oil, grapeseed oil, almond oil, hazelnut oil, macadamia nut oil, pumpkin seed oil, hemp seed oil, camelina oil, mustard oil, pistachio oil, pecan oil, poppy seed oil, black seed oil, apricot kernel oil, cherry kernel oil, peach kernel oil, tamanu oil, borage oil, evening primrose oil, sea buckthorn oil, perilla oil, moringa oil, neem oil, karanja oil, jojoba oil, tung oil, camellia oil, tea seed oil, babassu oil, murumuru oil, safflower oil, shea butter, cocoa butter, sal butter, kokum butter, mango butter, illipe butter, or fish oil.

42. The aqueous ultrasound coupling agent of any one of embodiments 1-40, wherein the at least one triglyceride is from soybean oil.

43. The aqueous ultrasound coupling agent of any one of embodiments 2, 9, 16, 23, and 31-42, comprising between 5% w/v and 9% w/v triglyceride.

44. The aqueous ultrasound coupling agent of any one of embodiments 1-6, 8-13, 15-20, 22-27, and 31-42, comprising about 7% w/v triglyceride.

45. The aqueous ultrasound coupling agent of any one of embodiments 1-38, comprising one or more triglycerides selected from any one of: tristearin, triolein, tripalmitin, triacetin, trilinolein, tricaprin, trilaurin, triarachidin, tridecanoin, trilinolenin, tricaprylin, tridecanoin, trimyristin, tricaprylin, trivalerate, tricaprate, trilinolenin, tridecanoate, triundecanoin, and trinonanoin.

46. The aqueous ultrasound coupling agent of any one of embodiments 1-38, comprising one or more triglycerides selected from any one of: linolenic-oleic-linolenic, linoleic-linoleic-linoleic, oleic-linoleic-oleic, linoleic-oleic-linoleic, linolenic-linoleic-I linolenic, palmitic-oleic-linoleic, palmitic-linoleic-palmitic, stearic-oleic-linoleic, linoleic-linolenic-linoleic, and linoleic-oleic-oleic.

47. The aqueous ultrasound coupling agent of any one of embodiments 1-40, comprising medium chain triglycerides.

48. The aqueous ultrasound coupling agent of any one of embodiments 1-7, 15-21, 29, and 31-47, comprising lecithin comprising the at least one phospholipid.

49. The aqueous ultrasound coupling agent of any one of embodiments 8-14, 22-28, and 30-48, wherein the lecithin comprises from about 20% to about 90% phospholipids by weight.

50. The aqueous ultrasound coupling agent of any one of embodiments 8-14, 22-28, and 30-48, wherein the lecithin comprises from about 60% to about 70% phospholipids by weight.

51. The aqueous ultrasound coupling agent of any one of embodiments 8-14, 22-28, and 30-50, wherein the lecithin is from one or more of soybeans, sunflower seeds, rapeseeds, egg yolks, wheat germ, corn, peanuts, sesame seeds, rice bran, flaxseeds, pumpkin seeds, cashews, almonds, hazelnuts, pistachios, walnuts, pecans, macadamia nuts, brazil nuts, chia seeds, hemp seeds, poppy seeds, quinoa, oats, barley, rye, millet, sorghum, buckwheat, amaranth, avocado, olives, cocoa beans, coconut, milk, butter, cheese, yogurt, beef liver, chicken liver, fish roe, mackerel, salmon, sardines, herring, anchovies, trout, halibut, tuna, and egg whites.

52. The aqueous ultrasound coupling agent of any one of embodiments 8-14, 22-28, and 30-50, wherein the lecithin is egg yolk lecithin.

53. The aqueous ultrasound coupling agent of any one of embodiments 8-14, 22-28, and 30-52, wherein the lecithin comprises phosphatidylcholine.

54. The aqueous ultrasound coupling agent of any one of embodiments 8-14, 22-28, and 30-52, wherein the lecithin comprises from about 10% to about 75% phosphatidylcholine by weight.

55. The aqueous ultrasound coupling agent of any one of embodiments 8-14, 22-28, and 30-52, wherein the lecithin comprises from about 20% to about 70% phosphatidylcholine by weight.

56. The aqueous ultrasound coupling agent of any one of embodiments 8-14, 22-28, and 30-52, wherein the lecithin comprises from about 60% to about 75% phosphatidylcholine by weight.

56.1 The aqueous ultrasound coupling agent of any one of embodiments 8-14, 22-28, and 30-52, wherein the lecithin comprises about 70% phosphatidylcholine, from 7 to about 10% phosphatidylethanolamine, and a maximum of 3% of lysophosphatidylcholine by weight of the lecithin.

56.2 The aqueous ultrasound coupling agent of any one of embodiments 8-14, 22-28, and 30-52, wherein the lecithin comprises at least 45% phosphatidylcholine, from 10 to about 18% phosphatidylethanolamine, and a maximum of 4% of lysophosphatidylcholine by weight of the lecithin.

56.3 The aqueous ultrasound coupling agent of any one of embodiments 8-14, 22-28, and 30-52, wherein the lecithin comprises about 92% phosphatidylcholine, from 0 to about 2% phosphatidylethanolamine, and a maximum of 3% of lysophosphatidylcholine by weight of the lecithin.

56.4 The aqueous ultrasound coupling agent of any one of embodiments 8-14, 22-28, and 30-52, wherein the lecithin comprises about 70% phosphatidylcholine, from about 12% to about 18% phosphatidylethanolamine, and a maximum of 3% of lysophosphatidylcholine by weight of the lecithin.

56.5 The aqueous ultrasound coupling agent of any one of embodiments 8-14, 22-28, and 30-52, wherein the lecithin comprises about 69.8% phosphatidylcholine, about 14.6% phosphatidylethanolamine, and about 1.5% of lysophosphatidylcholine by weight of the lecithin.

56.6 The aqueous ultrasound coupling agent of any one of embodiments 8-14, 22-28, and 30-52, wherein the lecithin comprises a minimum of 50% of phosphatidylcholine and a minimum of 5% phosphatidylethanolamine by weight of the lecithin.

56.7 The aqueous ultrasound coupling agent of any one of embodiments 8-14, 22-28, and 30-52, wherein the lecithin comprises a minimum of 20% of phosphatidylcholine and a minimum of 20% lysophosphatidyl choline by weight of the lecithin.

57. The aqueous ultrasound coupling agent of any one of embodiments 1-7, 15-21, 29, and 31-48, wherein the at least one phospholipid comprises phosphatidylcholine.

58. The aqueous ultrasound coupling agent of any one of embodiments 1-7, 15-21, 29, and 31-48, wherein the at least one phospholipid comprises from about 10% to about 70% phosphatidylcholine by weight.

59. The aqueous ultrasound coupling agent of any one of embodiments 1-7, 15-21, 29, and 31-48, wherein the at least one phospholipid comprises from about 20% to about 70% phosphatidylcholine by weight.

60. The aqueous ultrasound coupling agent of any one of embodiments 1-7, 15-21, 29, and 31-48, wherein the at least one phospholipid comprises from about 60% to about 70% phosphatidylcholine by weight.

61. The aqueous ultrasound coupling agent of any one of embodiments 9, 12, 23, 26, and 31-60, comprising from about 1 g/L to about 12 g/L lecithin.

62. The aqueous ultrasound coupling agent of any one of embodiments 9, 12, 23, 26, and 31-60, comprising from about 4.2 g/L to about 12 g/L lecithin.

63. The aqueous ultrasound coupling agent of any one of embodiments 9, 12, 23, 26, and 31-60, comprising up to about 12 g/L lecithin.

64. The aqueous ultrasound coupling agent of any one of embodiments 8-10, 12-13, 22-24, 26-27, and 31-60, comprising about 4.2 g/L lecithin.

65. The aqueous ultrasound coupling agent of any one of embodiments 9, 12, 23, 26, and 31-60, comprising from about 0.1% w/v to about 1.2% w/v lecithin.

66. The aqueous ultrasound coupling agent of any one of embodiments 9, 12, 23, 26, and 31-60, comprising from about 0.4% w/v to about 1.2% w/v lecithin.

67. The aqueous ultrasound coupling agent of any one of embodiments 9, 12, 23, 26, and 31-60, comprising up to about 1.2% w/v lecithin.

68. The aqueous ultrasound coupling agent of any one of embodiments 8-10, 12-13, 22-24, 26-27, and 31-60, comprising about 0.42% w/v lecithin.

65. The aqueous ultrasound coupling agent of any one of embodiments 2, 5, 16, 19, and 31-60, comprising from about 1 g/L to about 12 g/L phospholipids.

66. The aqueous ultrasound coupling agent of any one of embodiments 2, 5, 16, 19, and 31-60, comprising from about 4.2 g/L to about 12 g/L phospholipids.

67. The aqueous ultrasound coupling agent of any one of embodiments 2, 5, 16, 19, and 31-60, comprising up to about 12 g/L phospholipids.

68. The aqueous ultrasound coupling agent of any one of embodiments 1-3, 5-6, 15-17, 19-20, and 31-60 comprising about 4.2 g/L phospholipids.

69. The aqueous ultrasound coupling agent of any one of embodiments 2, 5, 16, 19, and 31-60, comprising from about 0.1% w/v to about 1.2% w/v phospholipids.

70. The aqueous ultrasound coupling agent of any one of embodiments 2, 5, 16, 19, and 31-60, comprising from about 0.4% w/v to about 1.2% w/v phospholipids.

71. The aqueous ultrasound coupling agent of any one of embodiments 2, 5, 16, 19, and 31-60, comprising up to about 1.2% w/v phospholipids.

72. The aqueous ultrasound coupling agent of any one of embodiments 1-3, 5-6, 15-17, 19-20, and 31-60, comprising about 0.42% w/v phospholipids.

73. The aqueous ultrasound coupling agent of any one of embodiments 1-72, wherein the pH is from about 6.5 to about 8.5.

74. The aqueous ultrasound coupling agent of any one of embodiments 1-72, wherein the pH is from about 6.9 to about 7.6.

75. The aqueous ultrasound coupling agent of any one of embodiments 1-72, wherein the pH is from about 6.9 to about 7.5.

76. The aqueous ultrasound coupling agent of any one of embodiments 1-72, wherein the pH is about 7.3.

77. The aqueous ultrasound coupling agent of any one of embodiments 1-72, wherein the pH is about 7.5.

78. The aqueous ultrasound coupling agent of any one of embodiments 1-72, wherein the pH is about 6.9.

79. The aqueous ultrasound coupling agent of any one of embodiments 1-78, wherein the osmolality is from about 270 mOsm/kg to about 330 mOsm/kg.

80. The aqueous ultrasound coupling agent of any one of embodiments 1-78, wherein the osmolality is from about 290 mOsm/kg to about 311 mOsm/kg.

81. The aqueous ultrasound coupling agent of any one of embodiments 1-78, wherein the osmolality is from about 290 mOsm/kg to about 305 mOsm/kg.

82. The aqueous ultrasound coupling agent of any one of embodiments 1-5, 8-12, 15-19, 22-26, and 31-81, comprising from about 14 g/L to about 26 g/L glycerol.

83. The aqueous ultrasound coupling agent of any one of embodiments 1-5, 8-12, 15-19, 22-26, and 31-81, comprising about 16 g/L glycerol.

84. The aqueous ultrasound coupling agent of any one of embodiments 1-6, 8-13, 15-20, 22-27, and 31-81 comprising about 23.7 g/L glycerol.

85. The aqueous ultrasound coupling agent of any one of embodiments 1-84, wherein the aqueous ultrasound coupling agent has a volume mean weighted droplet size of from about 0.2 μm to about 0.55 μm.

86. The aqueous ultrasound coupling agent of any one of embodiments 1-84, wherein the aqueous ultrasound coupling agent has a volume mean weighted droplet size of from about 0.26 μm to about 0.50 μm.

87. The aqueous ultrasound coupling agent of any one of embodiments 1-84, wherein the aqueous ultrasound coupling agent has a volume mean weighted droplet size of from about 228 nm to about 342 nm.

88. The aqueous ultrasound coupling agent of any one of embodiments 1-84, wherein the aqueous ultrasound coupling agent has a volume mean weighted droplet size of about 258 nm.

89. The aqueous ultrasound coupling agent of any one of embodiments 1-84, wherein the aqueous ultrasound coupling agent forms oil droplets, and wherein 100% of the oil droplets have a diameter that is less than 0.84 μm.

90. The aqueous ultrasound coupling agent of any one of embodiments 1-84, wherein the aqueous ultrasound coupling agent forms oil droplets, and wherein 100% of the oil droplets have a diameter that is less than 0.96 μm.

91. The aqueous ultrasound coupling agent of any one of embodiments 1-84, wherein the aqueous ultrasound coupling agent forms oil droplets, and wherein 100% of the oil droplets have a diameter that is less than 1.91 μm.

92. The aqueous ultrasound coupling agent of any one of embodiments 1-84, wherein the aqueous ultrasound coupling agent forms oil droplets, and wherein substantially none of the droplets comprise a diameter of greater than 5000 nm.

93. The aqueous ultrasound coupling agent of any one of embodiments 1-84, wherein the aqueous ultrasound coupling agent forms oil droplets, and wherein the oil droplets have an average size of from 200 nm to about 750 nm.

94. The aqueous ultrasound coupling agent of any one of embodiments 1-84, wherein the aqueous ultrasound coupling agent forms oil droplets, and wherein the oil droplets have an average size of from 250 nm to about 500 nm.

95. The aqueous ultrasound coupling agent of any one of embodiments 1-84, wherein the coupling agent has an average droplet size of about 285 nm±20%, wherein the average droplet size is measured by dynamic light scattering.

96. The aqueous ultrasound coupling agent of any one of embodiments 2, 9, 16, 23, and 31-95, comprising between about 50 g/L and about 90 g/L of at least one triglyceride.

97. The aqueous ultrasound coupling agent of any one of embodiments 1-6, 8-13, 15-20, 22-27, and 31-95, comprising about 70 g/L of at least one triglyceride.

98. The aqueous ultrasound coupling agent of any one of embodiments 1-86, wherein the shelf life of the aqueous ultrasound coupling agent is from about six months to about five years, from about 1 year to about 5 years, from about 2 years to about 5 years, from about 3 years to about 5 years, from about 6 months to about 4 years, from about 6 months to about 3 years, from about 6 months to about 2 years, from about six months to about one year, from about 1 year to about 4 years, from about 1 year to about 3 years, from about 1 year to about 2 years, or from about 2 years to about 4 years.

99. The aqueous ultrasound coupling agent of any one of embodiments 1-6, 15-20, and 31-98, wherein the ratio of the at least one triglyceride to the at least one phospholipid is about 16.7:1 by weight.

100. The aqueous ultrasound coupling agent of any one of embodiments 8-13, 22-27, and 31-98, wherein the ratio of the at least one triglyceride to the lecithin is about 16.7:1 by weight.

101. A method comprising
i) contacting an organ or a cavity with an ultrasound coupling agent of any one of embodiments 1-100;
ii) obtaining an ultra sound image of the organ or the cavity.

102. The method of embodiment 101, wherein the cavity is selected from any one of oral cavity, nasal cavity, orbital cavity, thoracic cavity, pericardial cavity, pleural cavities, abdominal cavity, pelvic cavity, cranial cavity, spinal cavity, articular cavities, synovial cavities, buccal cavity, cranial fossae, mandibular fossa, maxillary sinus, frontal sinus, ethmoidal sinus, sphenoidal sinus, mastoid air cells, auditory canal, middle ear cavity, tympanic cavity, vestibular system, cochlear duct, olfactory epithelium, oral vestibule, palatine tonsil, pharyngeal tonsil, lingual tonsil, sinus venosus, infundibulum, alveolar sacs, bronchioles, laryngeal cavity, tracheal cavity, gastric pits, crypts of lieberkühn, gallbladder fossa, renal sinus, ureteral opening, bladder trigone, ovarian follicles, testicular lobules, vaginal fornix, anal canal, vertebral foramen, intervertebral foramina, foramen magnum, and jugular foramen.

103. The method of embodiment 101, wherein the organ is selected from any one of brain, heart, lungs, liver, kidneys, stomach, small intestine, large intestine, pancreas, spleen, gallbladder, colon, spine, bladder, skin, eyes, ears, nose, mouth, tongue, throat, trachea, breast, esophagus, diaphragm, adrenal gland, thyroid, parathyroid gland, pituitary gland, pineal gland, hypothalamus, ovaries, testes, uterus, prostate, penis, vagina, cervix, urethra, rectum, anus, skeletal muscles, smooth muscle, cardiac muscle, bone marrow, bones, blood vessel, lymph node, lymphatic vessel, tonsils, appendix, adipose tissue, nerves, and connective tissue.

104. The method of any one of embodiments 103, wherein the organ or the cavity comprises a tumor.

105. The method of embodiment 104, comprising resecting the tumor.

106. The method of any one of embodiments 101-105, wherein the organ is the brain.

104. A container comprising an aqueous ultrasound coupling agent of any one of embodiments 1-100.

105. The container of embodiment 104, wherein the container is a syringe.

106. The container of embodiment 103 or 104, wherein the container is glass.

107. The container of embodiment 104 or 105, wherein the container is a bag.

108. The container of any one of embodiments 104-107, wherein the container lacks one or more of dyes, preservatives, rubber, butyl, silica, polyvinyl chloride (PVC), and di (2-ethylhexyl) phthalate (DEHP).

109. A kit comprising an aqueous ultrasound coupling agent of any one of embodiments 1-100 and a container.

110. The kit of embodiment 109, wherein the container is a syringe.

111. The kit of embodiment 109 or 110, wherein the container is glass.

112. The kit of embodiment 109, wherein the container is a bag.

113. The kit of any one of embodiments 109-112, wherein the container lacks one or more of dyes, preservatives, rubber, butyl, silica, polyvinyl chloride (PVC), and di (2-ethylhexyl) phthalate (DEHP).

114. A method of performing neurosurgery comprising contacting an instrument used for surgery with an aqueous ultrasound coupling agent of any one of embodiments 1-100 and inserting the instrument into a brain.

115. The method of embodiment 114, wherein the instrument is a catheter.

116. The method of embodiment 115, wherein the catheter is a subdural pressure cather, a drainage catheter, a cerebral flow catheter, a microdialysis catheter, or a brain tissue oxygen cathether.

117. The method of embodiment 114, wherein the instrument is an intracranial pressure sensor.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. An aqueous ultrasound coupling agent consisting essentially of:
(a) from about 60.0 g/L to about 80.0 g/L of at least one triglyceride;
(b) from about 3.2 g/L to about 5.2 g/L of at least one phospholipid;
(c) water;
(d) pH buffer; and
(e) glycerol.

2. An aqueous ultrasound coupling agent consisting essentially of:
(a) at least one triglyceride;
(b) at least one phospholipid;
(c) water,
(d) pH buffer; and
(e) glycerol; and
wherein the ratio of the at least one triglyceride to the at least one phospholipid is from about 13:1 to about 20:1 by weight.

3. An aqueous ultrasound coupling agent consisting essentially of:
(a) between 5% w/v and 9% w/v of at least one triglyceride;
(b) from about 0.2% w/v to about 1.2% w/v of at least one phospholipid;
(c) water;
(d) pH buffer; and
(e) glycerol.

4. An aqueous ultrasound coupling agent consisting essentially of:
(a) between 5% w/v and 9% w/v of at least one triglyceride;
(b) at least one phospholipid, wherein the at least one phospholipid is present at about 0.42% w/V;
(c) water;
(d) pH buffer; and
(e) glycerol.

5. An aqueous ultrasound coupling agent consisting essentially of:
(a) between 5% w/v and 9% w/v of at least one triglyceride;
(b) at least one phospholipid;
(c) water;
(d) pH buffer; and
(e) glycerol.

6. An aqueous ultrasound coupling agent consisting essentially of:
(a) between 5% w/v and 9% w/v of at least one triglyceride;
(b) from about 0.2% w/v to about 1.2% w/v of at least one phospholipid;
(c) water;
(d) pH buffer; and
(e) from about 21 g/L to about 26 g/L of glycerol.

7. An aqueous ultrasound coupling agent consisting essentially of:
(a) about 7% w/v of a triglyceride;
(b) about 4.2 g/L of a phospholipid;
(c) about 23.7 g/L of glycerol;
(d) water; and
(e) a pH buffer.

8. An aqueous ultrasound coupling agent consisting essentially of:
(a) about 7% w/v of a triglyceride;
(b) about 8.4 g/L of a phospholipid;
(c) glycerol;
(d) water; and
(e) a pH buffer;
wherein the aqueous ultrasound coupling agent has an osmolality of from about 270 mOsm/kg to about 330 mOsm/kg.

9. An aqueous ultrasound coupling agent consisting essentially of:
(a) about 7% w/v of a triglyceride;
(b) about 8.4 g/L of a phospholipid;
(c) from about 10 g/L to about 35 g/L glycerol;
(d) water; and
(e) a pH buffer.

10. An aqueous ultrasound coupling agent consisting essentially of:
(a) between 5% w/v and 9% w/v of a triglyceride;
(b) from about 7.4 g/L to about 9.4 g/L of a phospholipid;
(c) from about 10 g/L to about 35 g/L glycerol;
(d) water; and
(e) a pH buffer.

11. An aqueous ultrasound coupling agent consisting essentially of:
(a) between 5% and 9% w/v of a triglyceride;
(b) from about 7.4 g/L to about 9.4 g/L of a phospholipid;
(c) glycerol;
(d) water; and
(e) a pH buffer;
wherein the aqueous ultrasound coupling agent has an osmolality of from about 270 mOsm/kg to about 330 mOsm/kg.

12. An aqueous ultrasound coupling agent consisting essentially of:
(a) from about 60.0 to about 80.0 g/L of at least one triglyceride;
(b) from about 3.2 to about 5.2 g/L of lecithin;
(c) water;
(d) pH buffer; and
(e) glycerol.

13. An aqueous ultrasound coupling agent consisting essentially of:
(a) at least one triglyceride;
(b) lecithin;
(c) water,
(d) pH buffer; and
(e) glycerol; and
wherein the ratio of the at least one triglyceride to the at least one phospholipid is from about 13:1 to about 20:1 by weight.

14. An aqueous ultrasound coupling agent consisting essentially of:
(a) between 5% w/v and 9% w/v of at least one triglyceride;
(b) from about 0.2% w/v to about 1.2% w/v of lecithin;
(c) water;
(d) pH buffer; and
(e) glycerol.

15. An aqueous ultrasound coupling agent consisting essentially of:
(a) between 5% w/v and 9% w/v of at least one triglyceride;
(b) about 0.42% w/v lecithin;
(c) water;
(d) pH buffer; and
(e) glycerol.

16. An aqueous ultrasound coupling agent consisting essentially of:
(a) between 5% w/v and 9% w/v of at least one triglyceride;
(b) lecithin;
(c) water;
(d) pH buffer; and
(e) glycerol.

17. An aqueous ultrasound coupling agent consisting essentially of:
(a) between 5% w/v and 9% w/v of at least one triglyceride;
(b) from about 0.2% w/v to about 1.2% w/v of lecithin;
(c) water;
(d) pH buffer; and
(e) from about 21 g/L to about 26 g/L of glycerol.

18. An aqueous ultrasound coupling agent consisting essentially of:
(a) 7% w/v of a triglyceride;
(b) about 4.2 g/L lecithin;
(c) about 23.7 g/L of glycerol;
(d) water; and
(e) a pH buffer.

19. An aqueous ultrasound coupling agent consisting essentially of:
(a) about 7% w/v of a triglyceride;
(b) about 8.4 g/L of lecithin;
(c) glycerol;
(d) water; and
(e) a pH buffer;
wherein the aqueous ultrasound coupling agent has an osmolality of from about 270 mOsm/kg to about 330 mOsm/kg.

20. An aqueous ultrasound coupling agent consisting essentially of:
(a) about 7% w/v of a triglyceride;
(b) about 8.4 g/L of lecithin;
(c) from about 10 g/L to about 35 g/L glycerol;
(d) water; and
(e) a pH buffer.

21. An aqueous ultrasound coupling agent consisting essentially of:
(a) between 5% w/v and 9% w/v of a triglyceride;
(b) from about 7.4 g/L to about 9.4 g/L of lecithin;
(c) from about 10 g/L to about 35 g/L glycerol;
(d) water; and
(e) a pH buffer.

22. An aqueous ultrasound coupling agent consisting essentially of:
(a) between 5% and 9% w/v of a triglyceride;
(b) from about 7.4 g/L to about 9.4 g/L of lecithin;
(c) glycerol;
(d) water; and
(e) a pH buffer;
wherein the aqueous ultrasound coupling agent has an osmolality of from about 270 mOsm/kg to about 330 mOsm/kg.

23. An aqueous ultrasound coupling agent comprising:
(a) from about 60.0 to about 80.0 g/L of at least one triglyceride;
(b) from about 3.2 to about 5.2 g/L of at least one phospholipid;
(c) water;
(d) pH buffer; and
(e) glycerol;
wherein the coupling agent does not include a stabilizer and does not include sodium chloride.

24. An aqueous ultrasound coupling agent comprising:
(a) at least one triglyceride;
(b) at least one phospholipid;
(c) water;
(d) pH buffer; and
(e) glycerol; and
wherein the ratio of the at least one triglyceride to the at least one phospholipid is from about 13:1 to about 20:1 by weight;
wherein the coupling agent does not include a stabilizer and does not include sodium chloride.

25. An aqueous ultrasound coupling agent comprising:
(a) between 5% w/v and 9% w/v of at least one triglyceride;
(b) from about 0.2% w/v to about 1.2% w/v of at least one phospholipid;
(c) water;
(d) pH buffer; and
(e) glycerol;
wherein the coupling agent does not include a stabilizer and does not include sodium chloride.

26. An aqueous ultrasound coupling agent comprising:
(a) between 5% w/v and 9% w/v of at least one triglyceride;
(b) at least one phospholipid, wherein the at least one phospholipid is present at about 0.42% w/V;
(c) water;
(d) pH buffer; and
(e) glycerol;
wherein the coupling agent does not include a stabilizer and does not include sodium chloride.

27. An aqueous ultrasound coupling agent comprising:
(a) between 5% w/v and 9% w/v of at least one triglyceride;
(b) at least one phospholipid;
(c) water;
(d) pH buffer; and
(e) glycerol;
wherein the coupling agent does not include a stabilizer and does not include sodium chloride.

28. An aqueous ultrasound coupling agent comprising:
(a) between 5% w/v and 9% w/v of at least one triglyceride;
(b) from about 0.2% w/v to about 1.2% w/v of at least one phospholipid;
(c) water;
(d) pH buffer; and
(e) from about 21 g/L to about 26 g/L of glycerol;
wherein the coupling agent does not include a stabilizer and does not include sodium chloride.

29. An aqueous ultrasound coupling agent comprising:
(a) about 7% w/v of a triglyceride;
(b) about 4.2 g/L of a phospholipid;
(c) about 23.7 g/L of glycerol;
(d) water; and
(e) a pH buffer;
wherein the coupling agent does not include a stabilizer and does not include sodium chloride.

30. An aqueous ultrasound coupling agent comprising:
(a) about 7% w/v of a triglyceride;
(b) about 8.4 g/L of a phospholipid;
(c) glycerol;
(d) water; and
(e) a pH buffer;
wherein the aqueous ultrasound coupling agent has an osmolality of from about 270 mOsm/kg to about 330 mOsm/kg;
wherein the coupling agent does not include a stabilizer and does not include sodium chloride.

31. An aqueous ultrasound coupling agent comprising:
(a) about 7% w/v of a triglyceride;
(b) about 8.4 g/L of a phospholipid;
(c) from about 10 g/L to about 35 g/L glycerol;
(d) water; and
(e) a pH buffer;
wherein the coupling agent does not include a stabilizer and does not include sodium chloride.

32. An aqueous ultrasound coupling agent comprising:
(a) between 5% w/v and 9% w/v of a triglyceride;
(b) from about 7.4 g/L to about 9.4 g/L of a phospholipid;
(c) from about 10 g/L to about 35 g/L glycerol;
(d) water; and
(e) a pH buffer;
wherein the coupling agent does not include a stabilizer and does not include sodium chloride.

33. An aqueous ultrasound coupling agent comprising:
(a) between about 5% and about 9% w/v of a triglyceride;
(b) from about 7.4 g/L to about 9.4 g/L of a phospholipid;
(c) glycerol;
(d) water; and
(e) a pH buffer;
wherein the aqueous ultrasound coupling agent has an osmolality of from about 270 mOsm/kg to about 330 mOsm/kg;
wherein the coupling agent does not include a stabilizer and does not include sodium chloride.

34. An aqueous ultrasound coupling agent comprising:
(a) from about 60.0 to about 80.0 g/L of at least one triglyceride;
(b) from about 3.2 to about 5.2 g/L of lecithin;
(c) water;
(d) pH buffer; and
(e) glycerol;
wherein the coupling agent does not include a stabilizer and does not include sodium chloride.

35. An aqueous ultrasound coupling agent comprising:
(a) at least one triglyceride;
(b) lecithin;
(c) water,
(d) pH buffer; and
(e) glycerol; and
wherein the ratio of the at least one triglyceride to the at least one phospholipid is from about 13:1 to about 20:1 by weight;
wherein the coupling agent does not include a stabilizer and does not include sodium chloride.

36. An aqueous ultrasound coupling agent comprising:
(a) between 5% w/v and 9% w/v of at least one triglyceride;
(b) from about 0.2% w/v to about 1.2% w/v of lecithin;
(c) water;
(d) pH buffer; and
(e) glycerol;
wherein the coupling agent does not include a stabilizer and does not include sodium chloride.

37. An aqueous ultrasound coupling agent comprising:
(a) between 5% w/v and 9% w/v of at least one triglyceride;
(b) about 0.42% w/v lecithin;
(c) water;
(d) pH buffer; and
(e) glycerol;
wherein the coupling agent does not include a stabilizer and does not include sodium chloride.

38. An aqueous ultrasound coupling agent comprising:
(a) between 5% w/v and 9% w/v of at least one triglyceride;
(b) lecithin;
(c) water;
(d) pH buffer; and
(e) glycerol;
wherein the coupling agent does not include a stabilizer and does not include sodium chloride.

39. An aqueous ultrasound coupling agent comprising:
(a) between 5% w/v and 9% w/v of triglyceride;
(b) from about 0.2% w/v to about 1.2% w/v of at least one lecithin;
(c) water;
(d) pH buffer; and
(e) from about 21 g/L to about 26 g/L of glycerol;
wherein the coupling agent does not include a stabilizer and does not include sodium chloride

40. An aqueous ultrasound coupling agent comprising:
(a) about 7% w/v of a triglyceride;
(b) about 4.2 g/L lecithin;
(c) about 23.7 g/L of glycerol;
(d) water; and
(e) a pH buffer;
wherein the coupling agent does not include a stabilizer and does not include sodium chloride.

41. An aqueous ultrasound coupling agent comprising:
(a) about 7% w/v of a triglyceride;
(b) about 8.4 g/L of lecithin;
(c) glycerol;
(d) water; and
(e) a pH buffer;
wherein the aqueous ultrasound coupling agent has an osmolality of from about 270 mOsm/kg to about 330 mOsm/kg;
wherein the coupling agent does not include a stabilizer and does not include sodium chloride.

42. An aqueous ultrasound coupling agent comprising:
(a) about 7% w/v of a triglyceride;
(b) about 8.4 g/L of lecithin;
(c) from about 10 g/L to about 35 g/L glycerol;
(d) water; and
(e) a pH buffer;
wherein the coupling agent does not include a stabilizer and does not include sodium chloride.

43. An aqueous ultrasound coupling agent comprising:
(a) between about 5% w/v and about 9% w/v of a triglyceride;
(b) from about 7.4 g/L to about 9.4 g/L of lecithin;
(c) from about 10 g/L to about 35 g/L glycerol;
(d) water; and
(e) a pH buffer;
wherein the coupling agent does not include a stabilizer and does not include sodium chloride.

44. An aqueous ultrasound coupling agent comprising:
(a) between about 5% and about 9% w/v of a triglyceride;
(b) from about 7.4 g/L to about 9.4 g/L of lecithin;
(c) glycerol;
(d) water; and
(e) a pH buffer;
wherein the aqueous ultrasound coupling agent has an osmolality of from about 270 mOsm/kg to about 330 mOsm/kg;
wherein the coupling agent does not include a stabilizer and does not include sodium chloride.

45. An aqueous ultrasound coupling agent comprising:
(a) about 7% w/v of a triglyceride;
(b) about 4.2 g/L of at least one phospholipid;
(c) about 23.7 g/L of glycerol;
(d) water; and
(e) a pH buffer.

46. An aqueous ultrasound coupling agent comprising:
(a) about 7% w/v of a triglyceride;
(b) about 4.2 g/L lecithin;
(c) about 23.7 g/L of glycerol;
(d) water; and
(e) a pH buffer.

47. An aqueous ultrasound coupling agent comprising:
(a) about 7% w/v of a triglyceride;
(b) about 8.4 g/L of at least one phospholipid;
(c) glycerol;
(d) water; and
(e) a pH buffer;
wherein the aqueous ultrasound coupling agent has an osmolality of from about 270 mOsm/kg to about 330 mOsm/kg.

48. An aqueous ultrasound coupling agent comprising:
(a) about 7% w/v of a triglyceride;
(b) about 8.4 g/L lecithin;
(c) glycerol;
(d) water; and
(e) a pH buffer;
wherein the aqueous ultrasound coupling agent has an osmolality of from about 270 mOsm/kg to about 330 mOsm/kg.

49. The aqueous ultrasound coupling agent of any one of claims 23-46, wherein the stabilizer is polysorbate 80 (PS80).

50. The aqueous ultrasound coupling agent of any one of claims 1-49, wherein the ultrasound coupling agent has an attenuation coefficient of from about 0.15 dB/(MHz*cm) to about 0.60 dB/(MHz*cm).

51. The aqueous ultrasound coupling agent of any one of claims 1-49, wherein the ultrasound coupling agent has an attenuation coefficient of from about 0.002 dB/(MHz*cm) to about 1.0 dB/(MHz*cm).

52. The aqueous ultrasound coupling agent of any one of claims 1-49, wherein the ultrasound coupling agent has an attenuation coefficient from about 0.05 dB/(MHz*cm) to about 15 dB/(MHz*cm).

53. The aqueous ultrasound coupling agent of any one of claims 1-49, wherein the ultrasound coupling agent has an attenuation coefficient from about 0.05 dB/(MHz*cm) to about 1.8 dB/(MHz*cm).

54. The aqueous ultrasound coupling agent of any one of claims 1-49, wherein the ultrasound coupling agent has an attenuation coefficient that is from about 0.5 dB/(MHz*cm) to about 1.5 dB/(MHz*cm).

55. The aqueous ultrasound coupling agent of any one of claims 1-49, wherein the ultrasound coupling agent has an attenuation coefficient that is from about 3 dB/(MHz*cm) to about 15 dB/(MHz*cm).

56. The aqueous ultrasound coupling agent of any one of claims 1-49, wherein the ultrasound coupling agent has an attenuation coefficient that is from about 0.1 dB/(MHz*cm) to about 0.5 dB/(MHz*cm).

57. The aqueous ultrasound coupling agent of any one of claims 1-49, wherein the ultrasound coupling agent has an attenuation coefficient that is from about 0.1 dB/(MHz*cm) to about 1.5 dB/(MHz*cm).

58. The aqueous ultrasound coupling agent of any one of claims 1-49, wherein the ultrasound coupling agent has an attenuation coefficient that is substantially similar to an attenuation coefficient of bone, brain, lung, liver, kidney, heart, cerebrospinal fluid, a soft tissue, a bladder, or a colon.

59. The aqueous ultrasound coupling agent of any one of claims 1-58, wherein the at least one triglyceride is from one or more of olive oil, coconut oil, palm oil, canola oil, soybean oil, sunflower oil, corn oil, peanut oil, safflower oil, cottonseed oil, sesame oil, flaxseed oil, walnut oil, avocado oil, rice bran oil, grapeseed oil, almond oil, hazelnut oil, macadamia nut oil, pumpkin seed oil, hemp seed oil, camelina oil, mustard oil, pistachio oil, pecan oil, poppy seed oil, black seed oil, apricot kernel oil, cherry kernel oil, peach kernel oil, tamanu oil, borage oil, evening primrose oil, sea buckthorn oil, perilla oil, moringa oil, neem oil, karanja oil, jojoba oil, tung oil, camellia oil, tea seed oil, babassu oil, murumuru oil, safflower oil, shea butter, cocoa butter, sal butter, kokum butter, mango butter, illipe butter, or fish oil.

60. The aqueous ultrasound coupling agent of any one of claims 1-59, wherein the at least one triglyceride is from soybean oil.

61. The aqueous ultrasound coupling agent of any one of claims 2, 13, 24, 35, and 49-60, comprising between 5% w/v and 9% w/v triglyceride.

62. The aqueous ultrasound coupling agent of any one of claims 1-6, 10-17, 21-28, 32-39, 43-44, and 49-60, comprising about 7% w/v triglyceride.

63. The aqueous ultrasound coupling agent of any one of claims 1-62, comprising one or more triglycerides selected from any one of: tristearin, triolein, tripalmitin, triacetin, trilinolein, tricaprin, trilaurin, triarachidin, tridecanoin, trilinolenin, tricaprylin, tridecanoin, trimyristin, tricaprylin, trivalerate, tricaprate, trilinolenin, tridecanoate, triundecanoin, and trinonanoin.

64. The aqueous ultrasound coupling agent of any one of claims 1-62, comprising one or more triglycerides selected from any one of: linolenic-oleic-linolenic, linoleic-linoleic-linoleic, oleic-linoleic-oleic, linoleic-oleic-linoleic, linolenic-linoleic-linolenic, palmitic-oleic-linoleic, palmitic-linoleic-palmitic, stearic-oleic-linoleic, linoleic-linolenic-linoleic, and linoleic-oleic-oleic.

65. The aqueous ultrasound coupling agent of any one of claims 1-62, comprising medium chain triglycerides.

66. The aqueous ultrasound coupling agent of any one of claims 1-11, 23-33, 45, 47, and 49-65, comprising lecithin comprising the at least one phospholipid.

67. The aqueous ultrasound coupling agent of any one of claims 12-22, 34-44, 46, and 48-66, wherein the lecithin comprises from about 20% to about 90% phospholipids by weight.

68. The aqueous ultrasound coupling agent of any one of claims 12-22, 34-44, 46, and 48-66, wherein the lecithin comprises from about 60% to about 70% phospholipids by weight.

69. The aqueous ultrasound coupling agent of any one of claims 12-22, 34-44, 46, and 48-68, wherein the lecithin is from one or more of soybeans, sunflower seeds, rapeseeds, egg yolks, wheat germ, corn, peanuts, sesame seeds, rice bran, flaxseeds, pumpkin seeds, cashews, almonds, hazelnuts, pistachios, walnuts, pecans, macadamia nuts, brazil nuts, chia seeds, hemp seeds, poppy seeds, quinoa, oats, barley, rye, millet, sorghum, buckwheat, amaranth, avocado, olives, cocoa beans, coconut, milk, butter, cheese, yogurt, beef liver, chicken liver, fish roe, mackerel, salmon, sardines, herring, anchovies, trout, halibut, tuna, and egg whites.

70. The aqueous ultrasound coupling agent of any one of claims 12-22, 34-44, 46, and 48-68, wherein the lecithin is egg yolk lecithin.

71. The aqueous ultrasound coupling agent of any one of claims 12-22, 34-44, 46, and 48-68, wherein the lecithin comprises phosphatidylcholine.

72. The aqueous ultrasound coupling agent of any one of claims 12-22, 34-44, 46, and 48-68, wherein the lecithin comprises from about 10% to about 75% phosphatidylcholine by weight.

73. The aqueous ultrasound coupling agent of any one of claims 12-22, 34-44, 46, and 48-68, wherein the lecithin comprises from about 20% to about 70% phosphatidylcholine by weight.

74. The aqueous ultrasound coupling agent of any one of claims 12-22, 34-44, 46, and 48-68, wherein the lecithin comprises from about 60% to about 75% phosphatidylcholine by weight.

75. The aqueous ultrasound coupling agent of any one of claims 1-11, 23-33, 45, 47, and 49-74, wherein the at least one phospholipid comprises phosphatidylcholine.

76. The aqueous ultrasound coupling agent of any one of claims 1-11, 23-33, 45, 47, and 49-75, wherein the at least one phospholipid comprises from about 10% to about 70% phosphatidylcholine by weight.

77. The aqueous ultrasound coupling agent of any one of claims 1-11, 23-33, 45, 47, and 49-75, wherein the at least one phospholipid comprises from about 20% to about 70% phosphatidylcholine by weight.

78. The aqueous ultrasound coupling agent of any one of claims 1-11, 23-33, 45, 47, and 49-75, wherein the at least one phospholipid comprises from about 60% to about 70% phosphatidylcholine by weight.

79. The aqueous ultrasound coupling agent of any one of claims 13, 16, 35, 38, and 49-78 comprising from about 1 g/L to about 12 g/L lecithin.

80. The aqueous ultrasound coupling agent of any one of claims 13, 16, 35, 38, and 49-78, comprising from about 4.2 g/L to about 12 g/L lecithin.

81. The aqueous ultrasound coupling agent of any one of claims 13, 16, 35, 38, and 49-78, comprising up to about 12 g/L lecithin.

82. The aqueous ultrasound coupling agent of any one of claims 12-14, 16-17, 21-22, 34-36, 38-39, 43-44, and 49-78, comprising about 4.2 g/L or 8.4 g/L lecithin.

83. The aqueous ultrasound coupling agent of any one of claims 13, 16, 35, 38, and 49-78, comprising from about 0.1% w/v to about 1.2% w/v lecithin.

84. The aqueous ultrasound coupling agent of any one of claims 13, 16, 35, 38, and 49-78, comprising from about 0.4% w/v to about 1.2% w/v lecithin.

85. The aqueous ultrasound coupling agent of any one of claims 13, 16, 35, 38, and 49-78, comprising up to about 1.2% w/v lecithin.

86. The aqueous ultrasound coupling agent of any one of claims 12-14, 16-17, 21-22, 34-36, 38-39, 43-44, and 49-78, comprising about 0.42% w/v or 0.84% w/v lecithin.

87. The aqueous ultrasound coupling agent of any one of claims 2, 5, 24, 27, and 49-86 comprising from about 1 g/L to about 12 g/L phospholipids.

88. The aqueous ultrasound coupling agent of any one of claims 2, 5, 24, 27, and 49-86, comprising from about 4.2 g/L to about 12 g/L phospholipids.

89. The aqueous ultrasound coupling agent of any one of claims 2, 5, 24, 27, and 49-86, comprising up to about 12 g/L phospholipids.

90. The aqueous ultrasound coupling agent of any one of claims 1-3, 5-6, 10-11, 23-25, 27-28, 32-33, and 49-78 comprising about 4.2 g/L or about 8.4 g/L phospholipids.

91. The aqueous ultrasound coupling agent of any one of claims 2, 5, 24, 27, and 49-86, comprising from about 0.1% w/v to about 1.2% w/v phospholipids.

92. The aqueous ultrasound coupling agent of any one of claims 2, 5, 24, 27, and 49-86, comprising from about 0.4% w/v to about 1.2% w/v phospholipids.

93. The aqueous ultrasound coupling agent of any one of claims 2, 5, 24, 27, and 49-86, comprising up to about 1.2% w/v phospholipids.

94. The aqueous ultrasound coupling agent of any one of claims 1-3, 5-6, 10-11, 23-25, 27-28, 32-33, and 49-78, comprising about 0.42% w/v or about 0.84% w/v phospholipids.

95. The aqueous ultrasound coupling agent of any one of claims 1-94, wherein the pH is from about 6.5 to about 8.5.

96. The aqueous ultrasound coupling agent of any one of claims 1-94, wherein the pH is from about 6.9 to about 7.6.

97. The aqueous ultrasound coupling agent of any one of claims 1-94, wherein the pH is from about 6.9 to about 7.5.

98. The aqueous ultrasound coupling agent of any one of claims 1-94, wherein the pH is about 7.3.

99. The aqueous ultrasound coupling agent of any one of claims 1-94, wherein the pH is about 7.5.

100. The aqueous ultrasound coupling agent of any one of claims 1-94, wherein the pH is about 6.9.

101. The aqueous ultrasound coupling agent of any one of claims 1-7, 9-10, 12-18, 20-21, 23-29, 31-32, 34-40, 42-43, 45-46, and 49-100 wherein the osmolality is from about 270 mOsm/kg to about 330 mOsm/kg.

102. The aqueous ultrasound coupling agent of any one of claims 1-7, 9-10, 12-18, 20-21, 23-29, 31-32, 34-40, 42-43, 45-46, and 49-100, wherein the osmolality is from about 290 mOsm/kg to about 311 mOsm/kg.

103. The aqueous ultrasound coupling agent of any one of claims 1-7, 9-10, 12-18, 20-21, 23-29, 31-32, 34-40, 42-43, 45-46, and 49-100, wherein the osmolality is from about 290 mOsm/kg to about 305 mOsm/kg.

104. The aqueous ultrasound coupling agent of any one of claims 1-5, 8-16, 19-27, 30-38, 41-44, 47-103, comprising from about 14 g/L to about 26 g/L glycerol.

105. The aqueous ultrasound coupling agent of any one of claims 1-5, 8-16, 19-27, 30-38, 41-44, 47-104, comprising about 16 g/L glycerol.

106. The aqueous ultrasound coupling agent of any one of claims 1-6, 8-17, 19-28, 30-39, 41-44, and 47-104 comprising about 23.7 g/L glycerol.

107. The aqueous ultrasound coupling agent of any one of claims 1-106, wherein the aqueous ultrasound coupling agent has a volume mean weighted droplet size of from about 0.2 μm to about 0.55 μm.

108. The aqueous ultrasound coupling agent of any one of claims 1-106, wherein the aqueous ultrasound coupling agent has a volume mean weighted droplet size of from about 0.26 μm to about 0.50 μm.

109. The aqueous ultrasound coupling agent of any one of claims 1-106, wherein the aqueous ultrasound coupling agent has a volume mean weighted droplet size of from about 228 nm to about 342 nm.

110. The aqueous ultrasound coupling agent of any one of claims 1-106, wherein the aqueous ultrasound coupling agent has a volume mean weighted droplet size of about 258 nm.

111. The aqueous ultrasound coupling agent of any one of claims 1-106, wherein the aqueous ultrasound coupling agent forms oil droplets, and wherein 100% of the oil droplets have a diameter that is less than 0.84 μm.

112. The aqueous ultrasound coupling agent of any one of claims 1-106, wherein the aqueous ultrasound coupling agent forms oil droplets, and wherein 100% of the oil droplets have a diameter that is less than 0.96 μm.

113. The aqueous ultrasound coupling agent of any one of claims 1-106, wherein the aqueous ultrasound coupling agent forms oil droplets, and wherein 100% of the oil droplets have a diameter that is less than 1.91 μm.

114. The aqueous ultrasound coupling agent of any one of claims 1-106, wherein the aqueous ultrasound coupling agent forms oil droplets, and wherein substantially none of the droplets comprise a diameter of greater than 5000 nm.

115. The aqueous ultrasound coupling agent of any one of claims 1-106, wherein the aqueous ultrasound coupling agent forms oil droplets, and wherein the oil droplets have an average size of from 200 nm to about 750 nm.

116. The aqueous ultrasound coupling agent of any one of claims 1-106, wherein the aqueous ultrasound coupling agent forms oil droplets, and wherein the oil droplets have an average size of from 250 nm to about 500 nm.

117. The aqueous ultrasound coupling agent of any one of claims 1-106, wherein the coupling agent has an average droplet size of about 285 nm±20%, wherein the average droplet size is measured by dynamic light scattering.

118. The aqueous ultrasound coupling agent of any one of claims 2, 13, 24, 35, and 49-117, comprising between about 50 g/L and about 90 g/L of at least one triglyceride.

119. The aqueous ultrasound coupling agent of any one of claims 1-6, 10-17, 21-28, 32-39, 43-44, and 49-118, comprising about 70 g/L of at least one triglyceride.

120. The aqueous ultrasound coupling agent of any one of claims 1-119, wherein the shelf life of the aqueous ultrasound coupling agent is from about six months to about five years, from about 1 year to about 5 years, from about 2 years to about 5 years, from about 3 years to about 5 years, from about 6 months to about 4 years, from about 6 months to about 3 years, from about 6 months to about 2 years, from about six months to about one year, from about 1 year to about 4 years, from about 1 year to about 3 years, from about 1 year to about 2 years, or from about 2 years to about 4 years.

121. The aqueous ultrasound coupling agent of any one of claims 1-11, 23-33, 45, 47, and 49-120, wherein the ratio of the at least one triglyceride to the at least one phospholipid is about 16.7:1 by weight.

122. The aqueous ultrasound coupling agent of any one of claims 12-22, 34-44, 46, and 48-120, wherein the ratio of the at least one triglyceride to the lecithin is about 16.7:1 by weight.

123. A method comprising
i) contacting an organ or a cavity with an ultrasound coupling agent of any one of claims 1-122;
ii) obtaining an ultra sound image of the organ or the cavity.

124. The method of claim 123, wherein the cavity is selected from any one of oral cavity, nasal cavity, orbital cavity, thoracic cavity, pericardial cavity, pleural cavities, abdominal cavity, pelvic cavity, cranial cavity, spinal cavity, articular cavities, synovial cavities, buccal cavity, cranial fossae, mandibular fossa, maxillary sinus, frontal sinus, ethmoidal sinus, sphenoidal sinus, mastoid air cells, auditory canal, middle ear cavity, tympanic cavity, vestibular system, cochlear duct, olfactory epithelium, oral vestibule, palatine tonsil, pharyngeal tonsil, lingual tonsil, sinus venosus, infundibulum, alveolar sacs, bronchioles, laryngeal cavity, tracheal cavity, gastric pits, crypts of lieberkühn, gallbladder fossa, renal sinus, ureteral opening, bladder trigone, ovarian follicles, testicular lobules, vaginal fornix, anal canal, vertebral foramen, intervertebral foramina, foramen magnum, and jugular foramen.

125. The method of claim 123, wherein the organ is selected from any one of brain, heart, lungs, liver, kidneys, stomach, small intestine, large intestine, pancreas, spleen, gallbladder, colon, spine, bladder, skin, eyes, ears, nose, mouth, tongue, throat, trachea, breast, esophagus, diaphragm, adrenal gland, thyroid, parathyroid gland, pituitary gland, pineal gland, hypothalamus, ovaries, testes, uterus, prostate, penis, vagina, cervix, urethra, rectum, anus, skeletal muscles, smooth muscle, cardiac muscle, bone marrow, bones, blood vessel, lymph node, lymphatic vessel, tonsils, appendix, adipose tissue, nerves, and connective tissue.

126. The method of any one of claims 123-125, wherein the organ or the cavity comprises a tumor.

127. The method of claim 126, comprising resecting the tumor.

128. The method of any one of claims 123-127, wherein the organ is the brain.

129. A container comprising an aqueous ultrasound coupling agent of any one of claims 1-122.

130. The container of claim 129, wherein the container is a syringe.

131. The container of claim 129 or 130, wherein the container is glass.

132. The container of claim 129 or 130, wherein the container is a bag.

133. The container of any one of claims 129-132, wherein the container lacks one or more of dyes, preservatives, rubber, butyl, silica, polyvinyl chloride (PVC), and di (2-ethylhexyl) phthalate (DEHP).

134. A kit comprising an aqueous ultrasound coupling agent of any one of claims 1-122 and a container.

135. The kit of claim 134, wherein the container is a syringe.

136. The kit of claim 134 or 135, wherein the container is glass.

137. The kit of claim 134, wherein the container is a bag.

138. The kit of any one of claims 134-137, wherein the container lacks one or more of dyes, preservatives, rubber, butyl, silica, polyvinyl chloride (PVC), and di(2-ethylhexyl)phthalate (DEHP).

139. The kit of any one of claims 134-138, further comprising a bag spike.

140. The kit of any one of claims 134-139, further comprising a filter.

141. The kit of claim 140, wherein the filter has a pore size of about 1.2 μm.

142. A method of performing neurosurgery comprising contacting an instrument used for surgery with an aqueous ultrasound coupling agent of any one of claims 1-122 and inserting the instrument into a brain.

143. The method of claim 142, wherein the instrument is a catheter.

144. The method of claim 143, wherein the catheter is a subdural pressure catheter, a drainage catheter, a cerebral flow catheter, a microdialysis catheter, or a brain tissue oxygen catheter.

145. The method of claim 142, wherein the instrument is an intracranial pressure sensor.

146. The method of any one of claims 142-145, wherein the aqueous ultrasound coupling agent acts as a lubricant for the instrument.
